SEQUENCE LISTING (30 sequences)

SEQ ID NO: 1 - protein, 270 aa, synthetic construct
  REGION 1..270: note = Synthetic: IL33 polypeptide
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 2 - protein, 228 aa, synthetic construct
  REGION 1..228: note = Synthetic: IL33 polypeptide
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 3 - protein, 228 aa, synthetic construct
  REGION 1..228: note = Synthetic: IL33 polypeptide
MKPKMKYSTNKISTAKWKNTASKALCFKLGKSQQKAKEVCPMYFMKLRSGLMIKKEACYFRRETTKRPSLKTGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 4 - protein, 144 aa, synthetic construct
  REGION 1..144: note = Synthetic: IL33 polypeptide
MKPKMKYSTNKISTAKWKNTASKALCFKLGNKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 5 - protein, 266 aa, synthetic construct
  REGION 1..266: note = Synthetic: IL33 polypeptide
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 6 - protein, 252 aa, Homo sapiens
  REGION 1..252: note = misc_feature - human AREG
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 7 - protein, 120 aa, synthetic construct
  REGION 1..120: note = Synthetic: anti-AREG antibody heavy chain
EVQLQQSGAELVRSGASVKLSCTASGFNIKDSYMHWVKQRPEQGLEWIGWVDPDNGDTEYAPEFQGRATLTADTFSSTAYLQLTSLTSEDTAVYYCNAPSTYGHYGFAYWGQGTLVTVSA

SEQ ID NO: 8 - protein, 113 aa, synthetic construct
  REGION 1..113: note = Synthetic: anti-AREG antibody light chain
DIVMTQAAPSVPVTPGESVSISCRSSKSLLHSNGKAYLYWFLQRPGQSPQLLIYRMSNLASGVPDRFSGSGSGTAFTLRISRVEAEDVGVYYCMQHLEYPLTFGAGTKLELKR

SEQ ID NO: 9 - protein, 8 aa, synthetic construct
  REGION 1..8: note = Synthetic: heavy chain CDR1
GFNIKDSY

SEQ ID NO: 10 - protein, 8 aa, synthetic construct
  REGION 1..8: note = Synthetic: heavy chain CDR2
VDPDNGDT

SEQ ID NO: 11 - protein, 13 aa, synthetic construct
  REGION 1..13: note = Synthetic: heavy chain CDR3
NAPSTYGHYGFAY

SEQ ID NO: 12 - protein, 11 aa, synthetic construct
  REGION 1..11: note = Synthetic: light chain CDR1
KSLLHSNGKAY

SEQ ID NO: 13 - skipped

SEQ ID NO: 14 - protein, 9 aa, synthetic construct
  REGION 1..9: note = Synthetic: light chain CDR3
MQHLEYPLT

SEQ ID NO: 15 - protein, 123 aa, synthetic construct
  REGION 1..123: note = Synthetic: anti-AREG antibody heavy chain
EVQLQQSGAELVKPGASVKLSCTASGFNIKDTYMHWVKQRPEQGLEWIGRIDPANRSTKYDPKFQGKATITADTSSNTADLHLSSLTSEDTAVYYCARLYGDSVWYFDVWGAGTTVTVSSAKT

SEQ ID NO: 16 - protein, 109 aa, synthetic construct
  REGION 1..109: note = Synthetic: anti-AREG antibody light chain
QIVLTQSPAILSASPGEKVTMTCRAGSSVNYIHWYQQKPGSSPKPWIYATSNLASGVPARFSGSGSGTSYSLTISRVEAEDAATYYCQQWSGYPPMLTFGAGTKLELKR

SEQ ID NO: 17 - protein, 8 aa, synthetic construct
  REGION 1..8: note = Synthetic: heavy chain CDR1
GFNIKDTY

SEQ ID NO: 18 - protein, 8 aa, synthetic construct
  REGION 1..8: note = Synthetic: heavy chain CDR2
IDPANRST

SEQ ID NO: 19 - protein, 13 aa, synthetic construct
  REGION 1..13: note = Synthetic: heavy chain CDR3
ARLYGDSVWYFDV

SEQ ID NO: 20 - protein, 5 aa, synthetic construct
  REGION 1..5: note = Synthetic: light chain CDR1
SSVNY

SEQ ID NO: 21 - skipped

SEQ ID NO: 22 - protein, 11 aa, synthetic construct
  REGION 1..11: note = Synthetic: light chain CDR3
QQWSGYPPMLT

SEQ ID NO: 23 - protein, 117 aa, synthetic construct
  REGION 1..117: note = Synthetic: anti-AREG antibody heavy chain
EVQLVESGGDLVKPGGSLKLSCAASGFTFSNSGMXWFRLTPDKRLEWVATISSGSTYTFYPDTVKGRFIISRXNAKNTLYLQMSSLKSEDTAIYYCVREIWPVWGAGTTITVSSAKT

SEQ ID NO: 24 - protein, 109 aa, synthetic construct
  REGION 1..109: note = Synthetic: anti-AREG antibody light chain
QAVVTQESALSTSPGETVTLTCRSSTGAVTTSNYANWVQEKPDHLFTGLLGDTDNRPPGVPARFSGSLLGDKAALTITGAQTEDEAIYFCALWYSNHWVFGGGTKLTVL

SEQ ID NO: 25 - protein, 8 aa, synthetic construct
  REGION 1..8: note = Synthetic: heavy chain CDR1
GFTFSNSG

SEQ ID NO: 26 - protein, 8 aa, synthetic construct
  REGION 1..8: note = Synthetic: heavy chain CDR2
ISSGSTYT

SEQ ID NO: 27 - protein, 7 aa, synthetic construct
  REGION 1..7: note = Synthetic: heavy chain CDR3
VREIWPV

SEQ ID NO: 28 - protein, 9 aa, synthetic construct
  REGION 1..9: note = Synthetic: light chain CDR1
TGAVTTSNY

SEQ ID NO: 29 - skipped

SEQ ID NO: 30 - protein, 9 aa, synthetic construct
  REGION 1..9: note = Synthetic: light chain CDR3
ALWYSNHWV

(19) United States
(12) Patent Application Publication
(10) Pub. No.: US 2024/0307493 A1
(43) Pub. Date: Sep. 19, 2024

(54) COMPOSITIONS AND METHODS FOR TREATING CANCER

(71) Applicant: UNIVERSITY OF PITTSBURGH - OF THE COMMONWEALTH SYSTEM OF HIGHER EDUCATION, Pittsburgh, PA (US)

(72) Inventors: Binfeng Lu, Clifton, NJ (US); Runzi Sun, Shanghai (CN)

(73) Assignee: UNIVERSITY OF PITTSBURGH - OF THE COMMONWEALTH SYSTEM OF HIGHER EDUCATION, Pittsburgh, PA (US)

(21) Appl. No.: 18/738,964

(22) Filed: Jun. 10, 2024

Related U.S. Application Data
(63) Continuation of application No. PCT/US2022/053135, filed on Dec. 16, 2022.
(60) Provisional application No. 63/296,288, filed on Jan. 4, 2022.

(51) Int. Cl. A61K 38/20; A61K 39/395; A61P 35/00

(52) U.S. Cl. CPC A61K 38/20; A61K 39/3955; A61P 35/00

(57) ABSTRACT

The present disclosure relates to the methods of preventing and/or treating cancer including administering IL33 and anti-AREG antibodies or fragments thereof to a subject in need thereof. The present disclosure further provides compositions and kits for performing such methods.

### CROSS-REFERENCE TO RELATED APPLICATIONS

This application is a continuation of International Patent Application No. PCT/US2022/053135, filed on Dec. 16, 2022, which claims priority to U.S. Provisional Patent Application Ser. No. 63/296,288, filed on Jan. 4, 2022, the contents of each of which are hereby incorporated by reference in their entireties, and to each of which priority is claimed.

### GRANT INFORMATION

The invention was made with government support under Grant No. #CA254274 awarded by National Institutes of Health. The government has certain rights to this disclosure.

### SEQUENCE LISTING

This application contains a Sequence Listing, which has been submitted in XML format via EFS-Web and is hereby incorporated by reference in its entirety. Said XML copy, created on Jun. 4, 2024, is named 072396_1016_SLST26.xml and is 37,590 bytes in size.

TECHNICAL FIELD

The presently disclosed subject matter provides methods, compositions, and kits for treating cancer.

### BACKGROUND OF THE INVENTION

While immunotherapies like immune checkpoint blockade with anti-PD-1 or anti-CTLA-4 antibodies have entered the mainstream of cancer treatment, these therapies as single modalities or even in combination with each other only benefit a subset of cancer patients. There is a growing body of literature showing that patients who had previously responded to immune checkpoint inhibitors can develop resistance to the immune checkpoint inhibitors later (Barrueto et al., Translational oncology vol. 13,3 (2020): 100738).

Thus, there remain needs for the development of novel methods and compositions for treating cancer and to improve patients' responsiveness to immunotherapies.

### SUMMARY OF THE INVENTION

The present disclosure provides a pharmaceutical composition comprising an IL33 polypeptide and an anti-AREG antibody or a fragment thereof. In certain embodiments, the pharmaceutical compositions is for use in treating and/or preventing a cancer in a subject in need thereof.

In certain embodiments, the IL33 polypeptide is recombinant. In certain embodiments, the IL33 polypeptide is a human IL33 polypeptide. In certain embodiments, the IL33 polypeptide comprises an amino acid sequence at least about 80% identical to the amino acid sequence set forth in SEQ ID NO: 1. In certain embodiments, the IL33 polypeptide comprises the amino acid sequence set forth in SEQ ID NO: 1. In certain embodiments, the IL33 polypeptide comprises an amino acid sequence comprising from amino acid 95 to amino acid 270 of SEQ ID NO: 1. In certain embodiments, the IL33 polypeptide comprises an amino acid sequence comprising from amino acid 99 to amino acid 270 of SEQ ID NO: 1. In certain embodiments, the IL33 polypeptide comprises an amino acid sequence comprising from amino acid 109 to amino acid 270 of SEQ ID NO: 1.

In certain embodiments, the IL-33 polypeptide is encoded by a vector. In certain embodiments, the vector is a viral vector. In certain embodiments, the vector is a retroviral vector. In certain embodiments, the vector is a γ-retroviral vector. In certain embodiments, the cancer is selected from the group consisting of adenocarcinomas, osteosarcomas, cervical carcinomas, melanomas, hepatocellular carcinomas, breast cancers, lung cancers, prostate cancers, ovarian cancers, leukemia, lymphomas, renal carcinomas, pancreatic cancers, gastric cancers, colon cancers, duodenal cancers, glioblastoma multiforme, astrocytomas, sarcomas, and combinations thereof. In certain embodiments, the cancer is selected from the group consisting of colon cancer, gastric cancer, breast cancer, lung cancer, pancreatic cancer, head and neck cancer, ovarian cancer, melanoma, and combinations thereof.

In certain embodiments, the subject is a human subject. In certain embodiments, the subject has received or is receiving an immunomodulatory agent. In certain embodiments, the immunomodulatory agent is an immune checkpoint inhibitor. In certain embodiments, the immune checkpoint inhibitor is selected from the group consisting of anti-PD1 antibodies, anti-PDL1 antibodies, anti-CTLA4 antibodies, anti-TLA antibodies, anti-TIM3 antibodies, anti-LAG3 antibodies, and any combinations thereof.

The present disclosure also provides a method for treating and/or preventing a cancer in a subject in need thereof. In certain embodiments, the present disclosure further provides for a method for inhibiting tumor growth in a subject in need thereof. In certain embodiments, the method comprises administering a therapeutically effective amount of an IL33 polypeptide and an anti-AREG antibody or a fragment thereof to the subject.

In certain embodiments, the IL33 polypeptide is recombinant. In certain embodiments, the IL33 polypeptide is human. In certain embodiments, the IL33 polypeptide comprises an amino acid sequence at least about 80% identical to the amino acid sequence set forth in SEQ ID NO: 1. In certain embodiments, the IL33 polypeptide comprises the amino acid sequence set forth in SEQ ID NO: 1. In certain embodiments, the IL33 polypeptide comprises an amino acid sequence comprising from amino acid 95 to amino acid 270 of SEQ ID NO: 1. In certain embodiments, wherein the IL33 polypeptide comprises an amino acid sequence comprising from amino acid 99 to amino acid 270 of SEQ ID NO: 1. In certain embodiments, the IL33 polypeptide comprises an amino acid sequence comprising from amino acid 109 to amino acid 270 of SEQ ID NO: 1.

In certain embodiments, the IL-33 polypeptide is encoded by a vector. In certain embodiments, wherein the vector is a viral vector. In certain embodiments, the vector is a retroviral vector. In certain embodiments, the vector is a γ-retroviral vector.

In certain embodiments, the cancer is selected from the group consisting of adenocarcinomas, osteosarcomas, cervical carcinomas, melanomas, hepatocellular carcinomas, breast cancers, lung cancers, prostate cancers, ovarian cancers, leukemia, lymphomas, renal carcinomas, pancreatic cancers, gastric cancers, colon cancers, duodenal cancers, glioblastoma multiforme, astrocytomas, sarcomas, and combinations thereof. In certain embodiments, the cancer is selected from the group consisting of colon cancer, gastric cancer, breast cancer, lung cancer, pancreatic cancer, head and neck cancer, ovarian cancer, melanoma, and combinations thereof. In certain embodiments, the subject is a human subject.

In certain embodiments, the method further comprises administering an immunomodulatory agent to the subject. In certain embodiments, the immunomodulatory agent is an immune checkpoint inhibitor. In certain embodiments, the immune checkpoint inhibitor is selected from the group consisting of anti-PD1 antibodies, anti-PDL1 antibodies, anti-CTLA4 antibodies, anti-BTLA antibodies, anti-TIM3 antibodies, anti-LAG3 antibodies, and any combinations thereof.

In certain embodiments, the IL33 polypeptide and an anti-AREG antibody or a fragment thereof show synergistic activity.

In certain embodiments, the IL33 polypeptide and an anti-AREG antibody or a fragment thereof are to be administered in one formulation or in alternation. In certain embodiments, the IL33 polypeptide and an anti-AREG antibody or a fragment thereof are to be administered simultaneously. In certain embodiments, the IL33 polypeptide and an anti-AREG antibody or a fragment thereof are to be administered consecutively. In certain embodiments, the tumor is resistant to treatment with said IL33 polypeptide or functional fragment thereof when administered as a single agent.

In certain embodiments, the tumor is an IL-33 positive tumor.

The present disclosure also provides a kit for use in treating and/or preventing a cancer in a subject in need thereof. In certain embodiments, the kit comprises an IL33 polypeptide and an anti-AREG antibody or a fragment thereof.

### BRIEF DESCRIPTION OF THE DRAWINGS

FIGS. 1A-1I show that IL-33 expression in the tumors drove robust CD8⁺ T cell immune responses. FIG. 1A shows a UMAP dimensionality reduction project for CD8⁺ T cells from B16 and B16-IL33 tumors to 2 dimensions showing 6 subclusters differentiated by color and trajectory analysis of different CD8 clusters. Each point represents a single cell, with cells of similar gene expression profiles positioned closer together in the projection. FIG. 1B shows a UMAP projection comparing the distribution of CD8⁺ T cells in B16-CON and B16-IL33 tumors. FIG. 1C depicts a violin plot showing the expression of the top marker gene for all 6 clusters based on adjusted P-value and log2FC. FIG. 1D depicts a bar plot showing the percentage of cells in each CD8⁺ T cell cluster based on tumor-origin (B16-CON vs B16-IL33). FIG. 1E depicts a bar plot showing the total unique T cell receptor (TCR) clones in all CD8 clusters from B16-CON or B16-IL33. FIG. 1F depicts a bar plot showing the percentage of clonally expanded cells in each CD8⁺ T T cell cluster. FIG. 1G depicts a bar plot showing the average clonal size in each CD8⁺ T cell cluster. FIG. 1H depicts the distribution of two representative CD8 clones on the UMAP plot. FIG. 1I shows a scatter plot comparing the percent of clonal cells (x) and percent of shared clonal cells in each cluster (y); the heatmap in the right depicts the percent of clonal cells shared between clusters.

FIGS. 2A-2F show that IL1RL1+ Treg cells increased in IL-33-expressing tumors. FIG. 2A shows UMAP dimensionality reduction projects for Treg cells from B16 and B16-IL33 tumors to 2 dimensions showing 6 subclusters differentiated by color and trajectory analysis of different CD8 clusters. Each point represents a single cell, with cells of similar gene expression profiles positioned closer together in the projection. FIG. 2B shows a UMAP projection comparing the distribution of Treg cells in B16-CON and B16-IL33 tumors. FIG. 2C depicts a dot plot showing the expression of the top marker gene for all 6 clusters based on adjusted P-value and log2FC. FIG. 2D depicts a bar plot showing the percentage of cells in each Treg cell cluster based on tumor-origin (B16-CON vs B16-IL33). FIG. 2E depicts a bar plot showing the average clonal size in Treg cells in B16-CON or B16-IL33 tumors. FIG. 2F depicts a bar plot showing the percentage of clonally expanded cells in each Treg cell cluster.

FIGS. 3A-3K show that specific deletion of on Il1rl1 in Tregs altered lymphocyte population in the TME. FIG. 3A shows a schematic demonstration for the strategy of generation of Foxp3^{cre}Il1rl1^{flox/flox} mice. FIG. 3B shows tumor size of B16-IL33 tumor bearing mice. FIG. 3C shows survival of B16-IL33 tumor bearing mice. For FIGS. 3B and 3C, B16-IL33 tumor cells (1×10⁵) inoculated i.d. into the right flank of the CON(Foxp3^{cre}) and condition knock-out (CKO) (Foxp3^{creIl}1r1^{flox/flox}) mice and tumor size was monitored every two days. FIG. 3D shows a representative flow cytometry plot and quantitative plot showing the percentage and number of

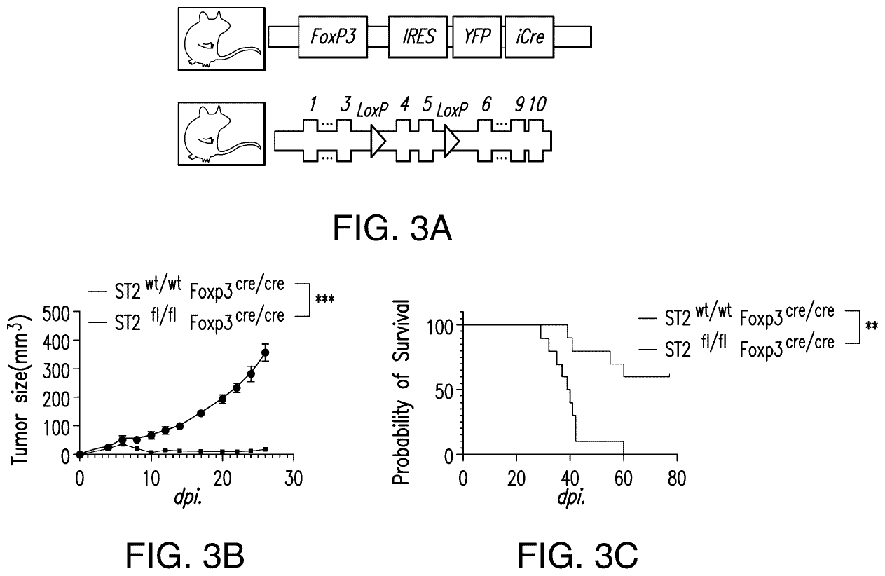

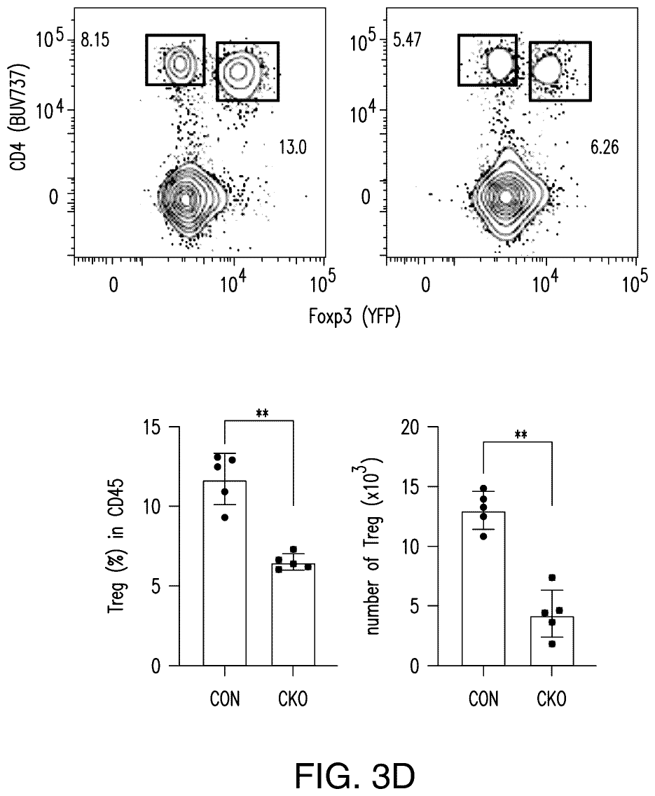

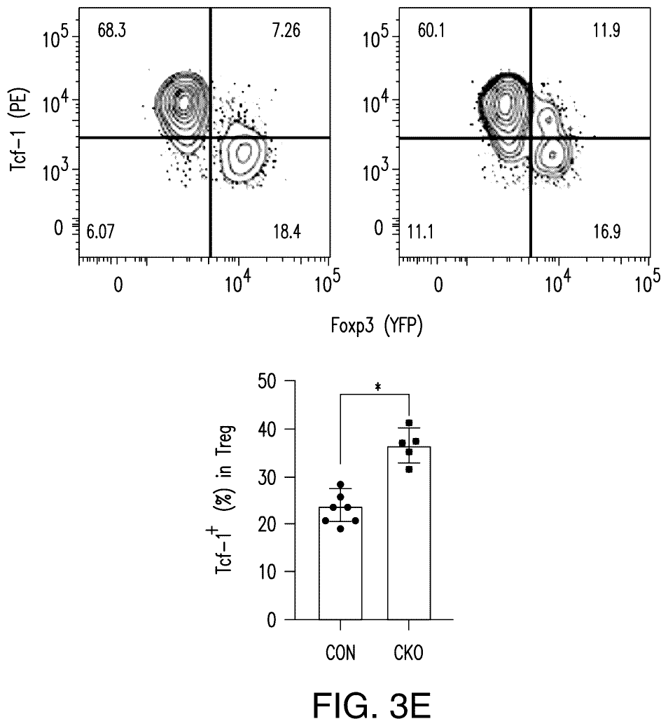

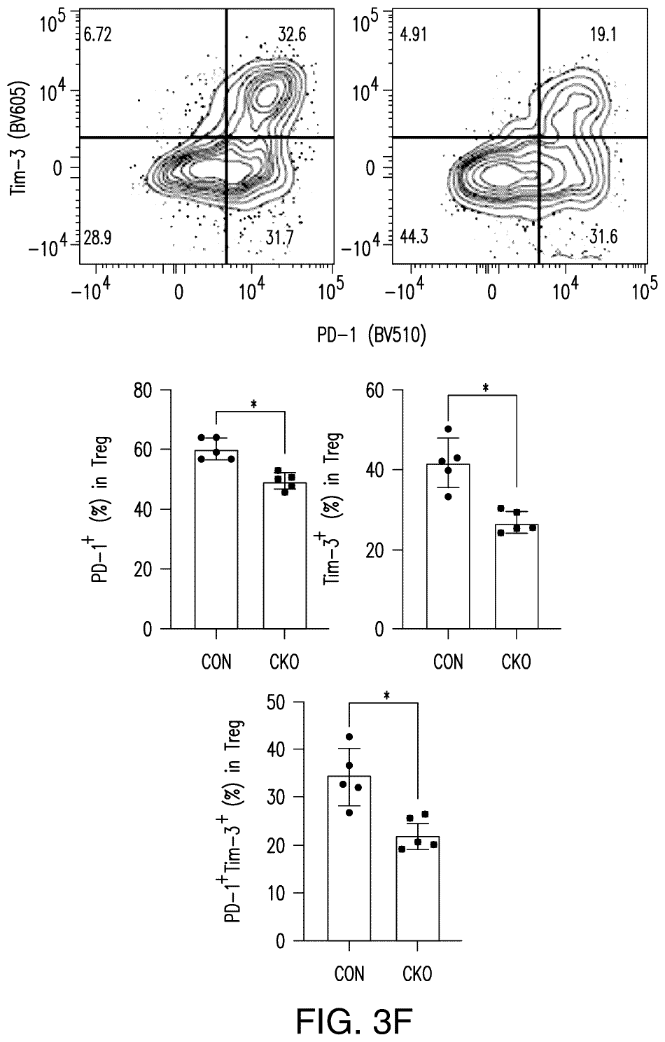

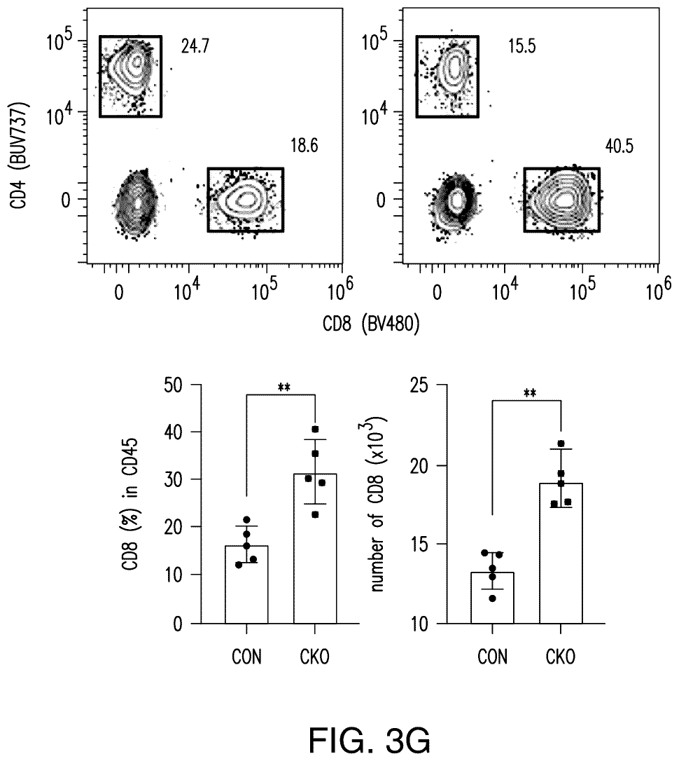

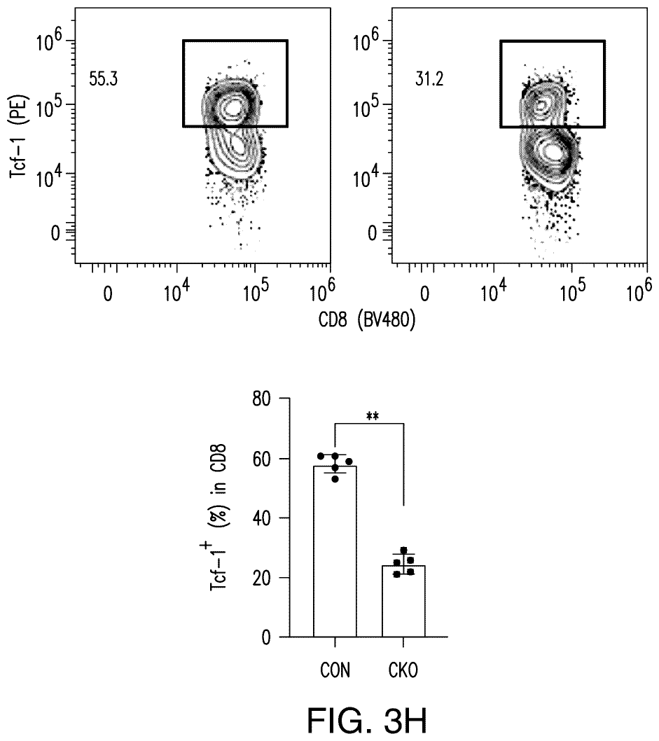

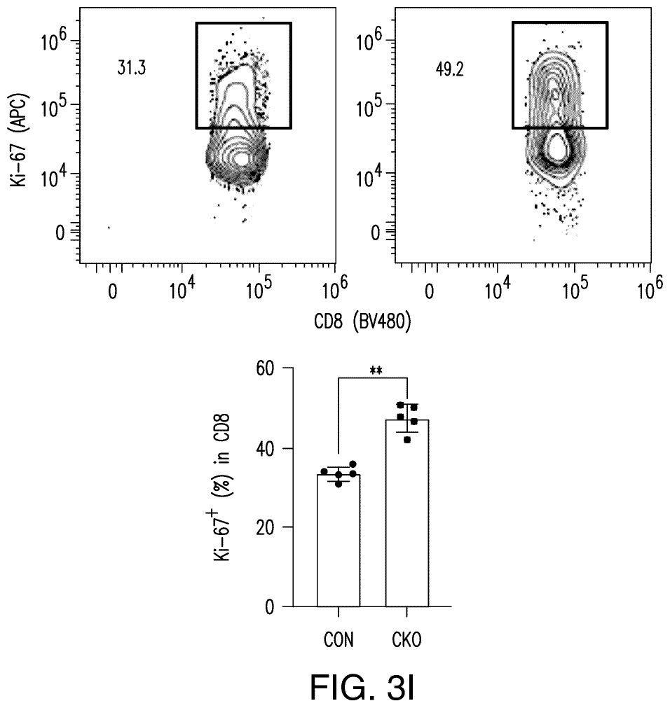

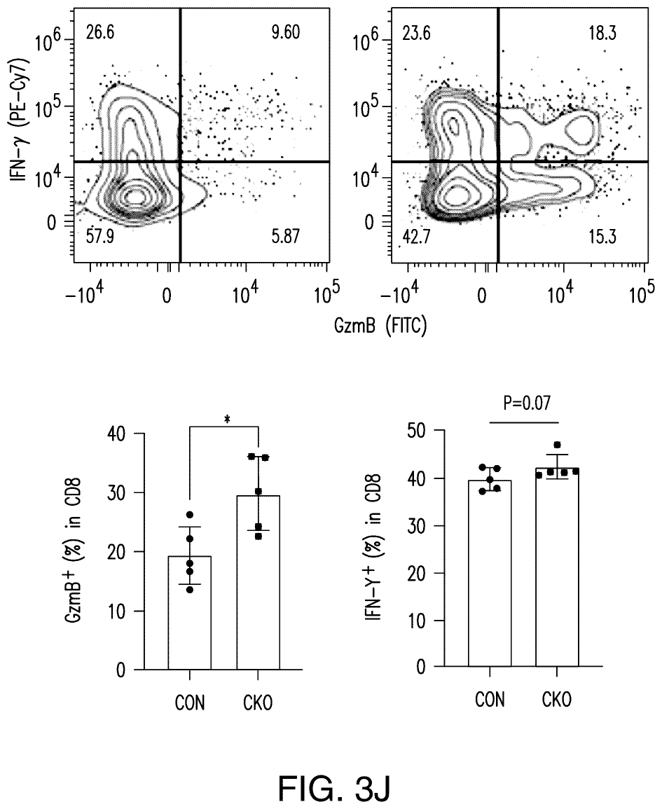

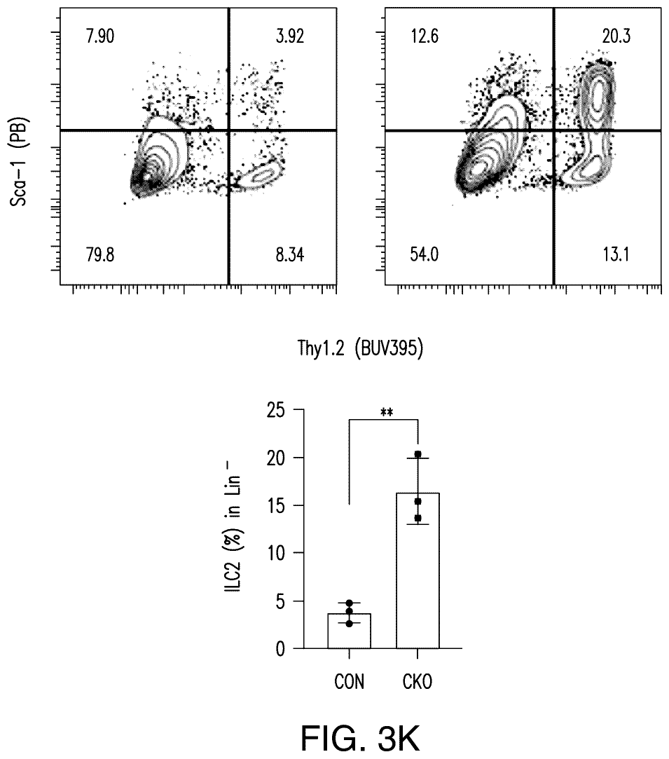

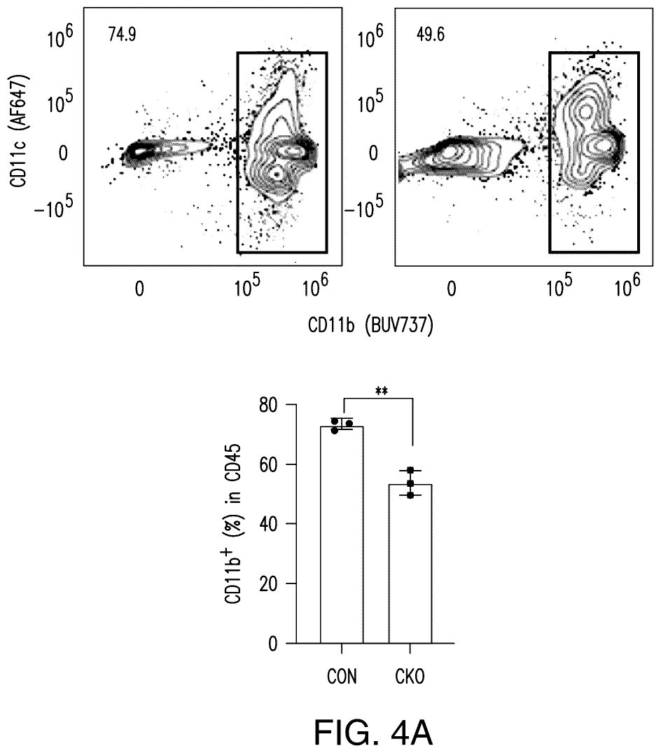

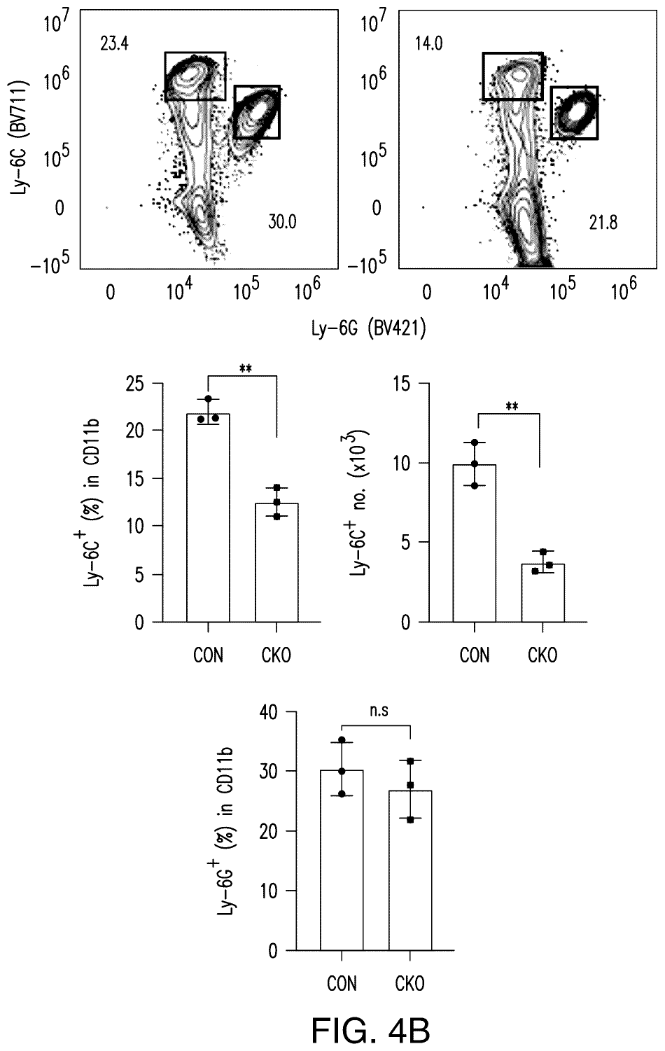

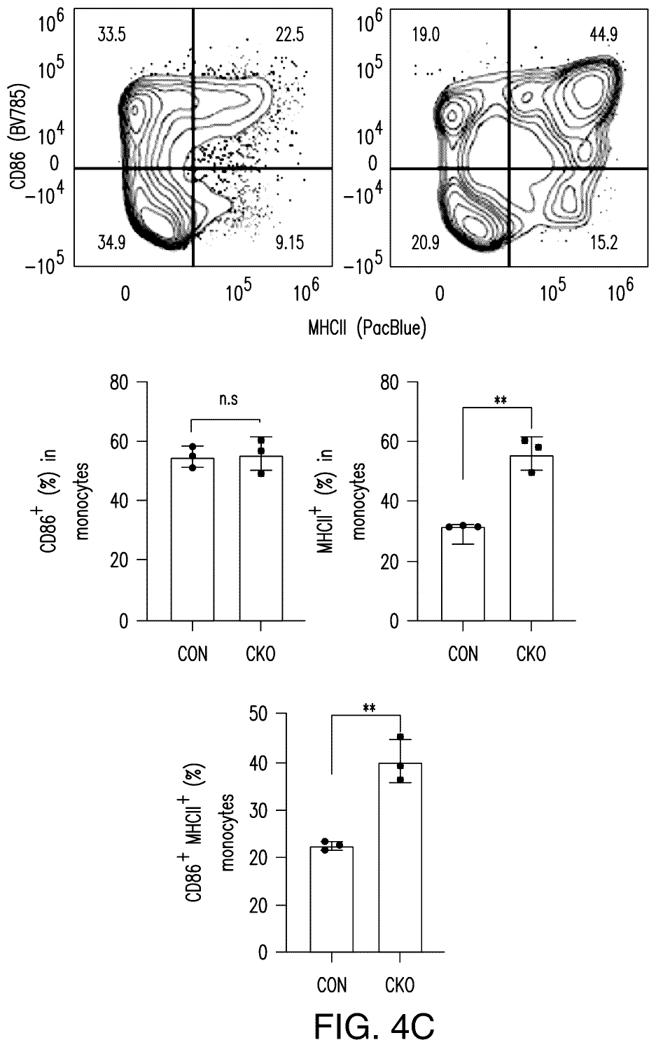

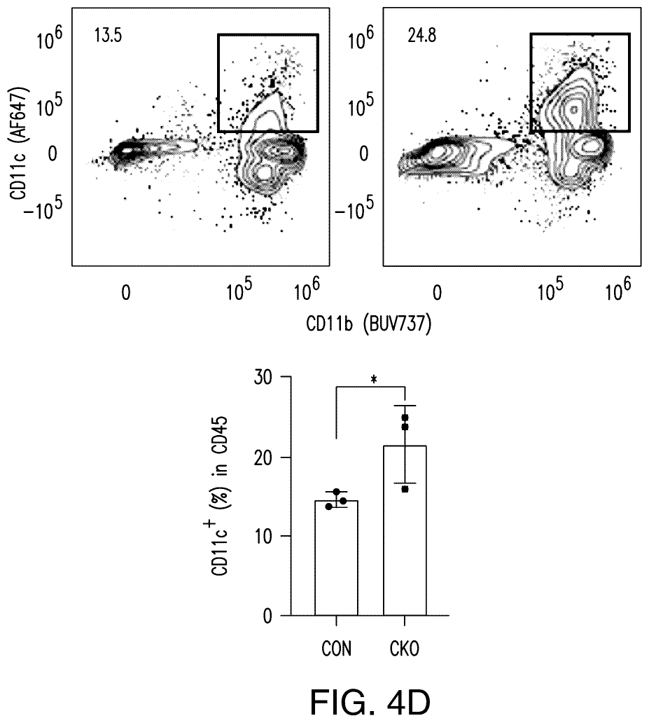

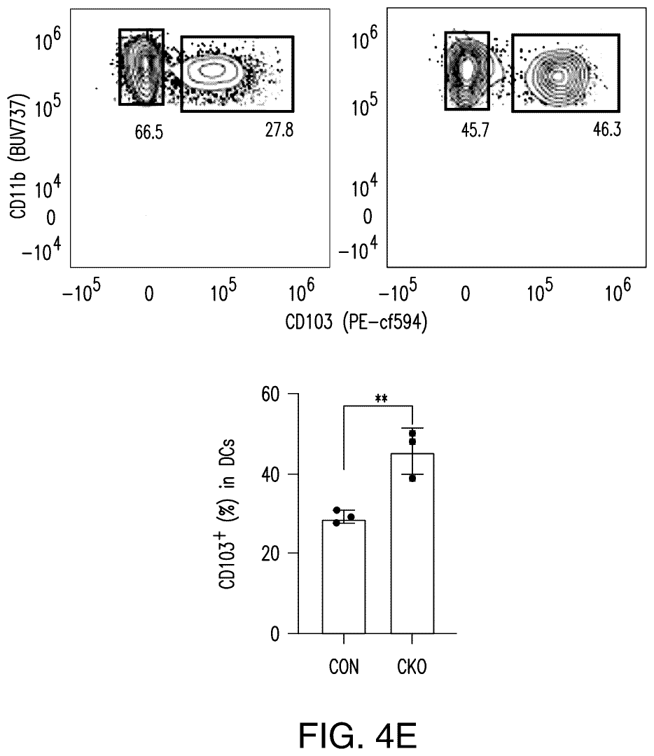

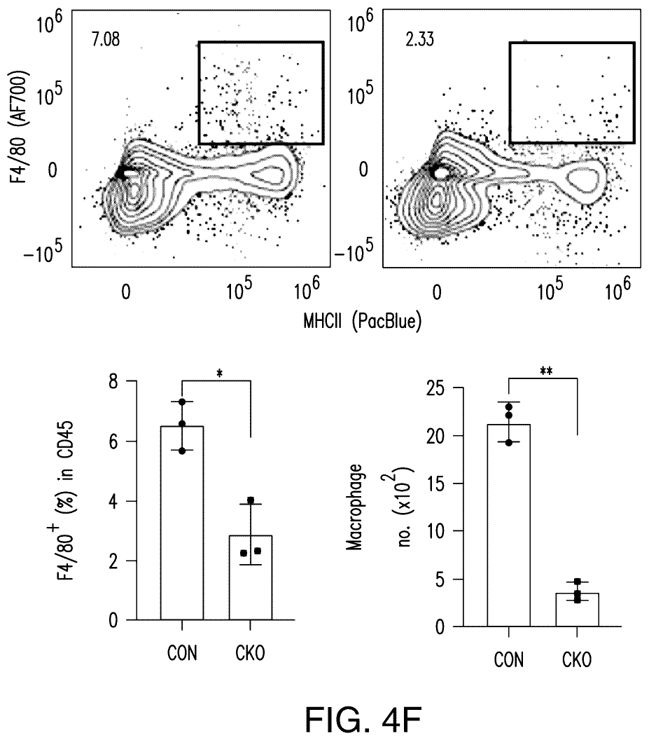

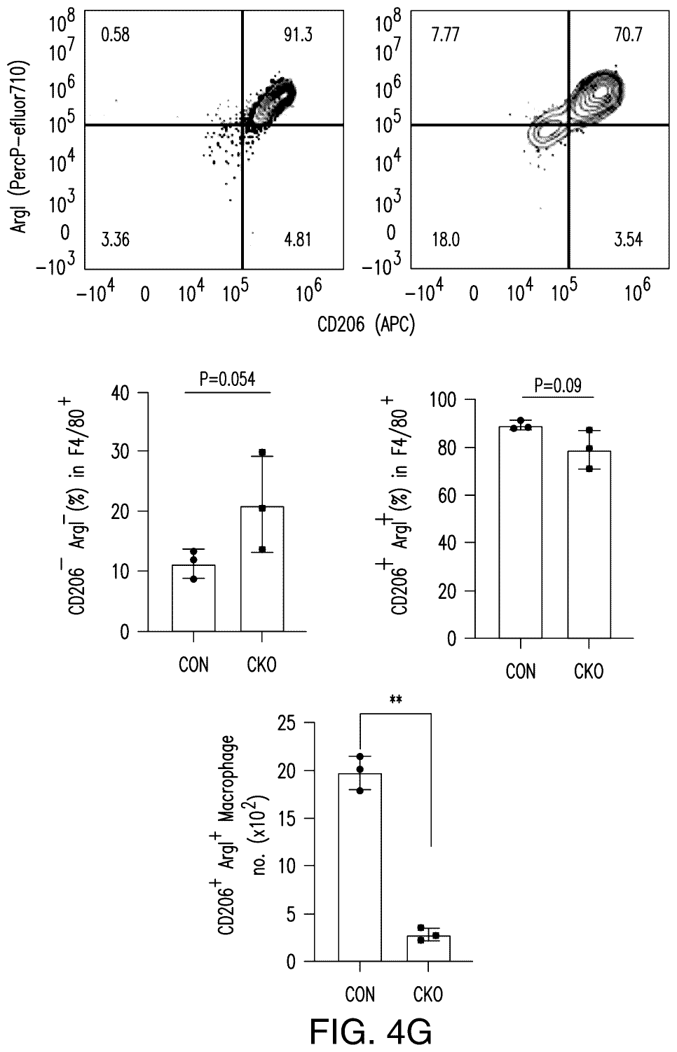

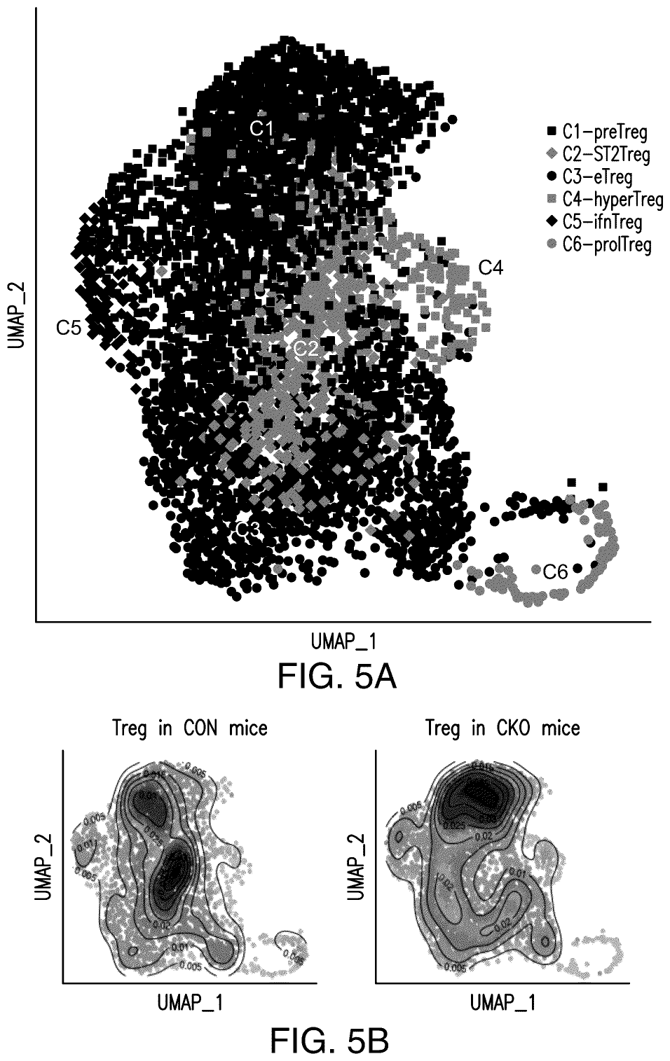

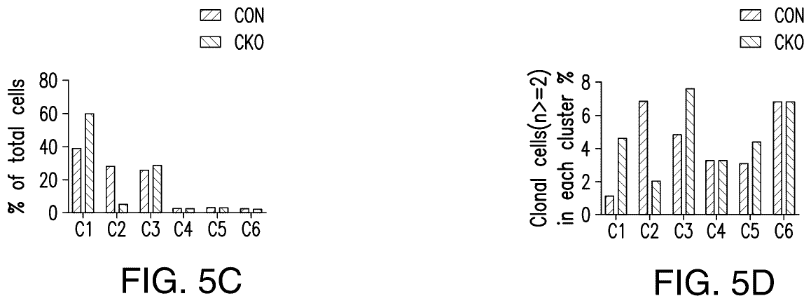

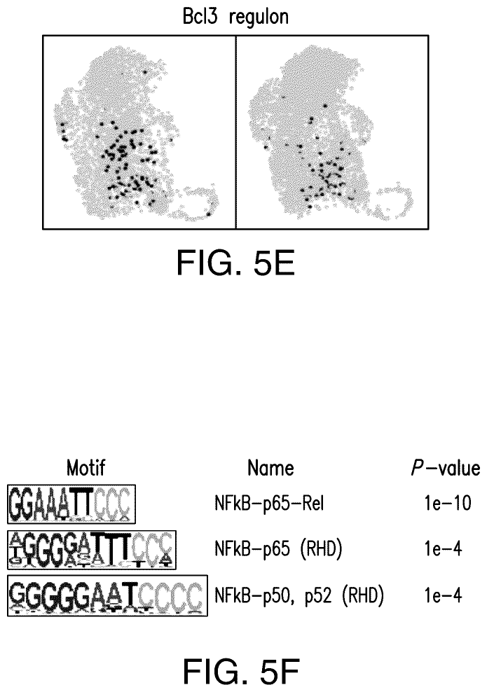

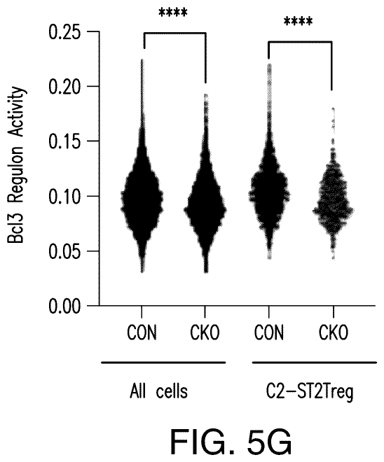

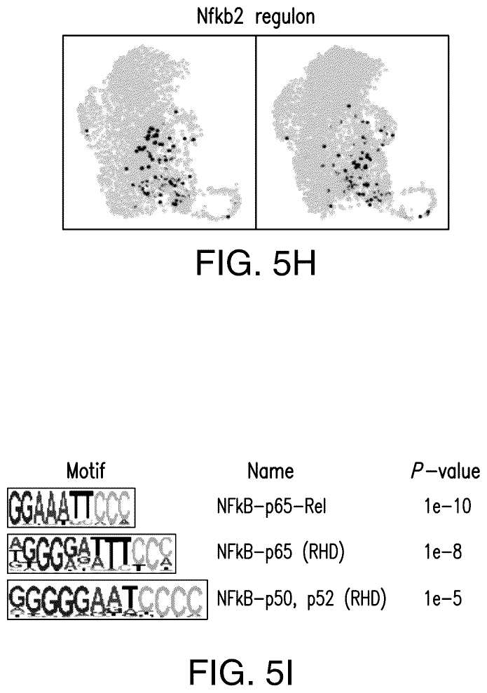

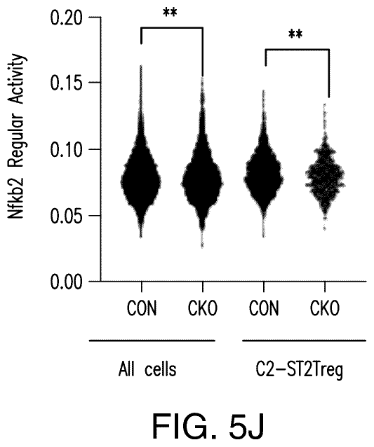

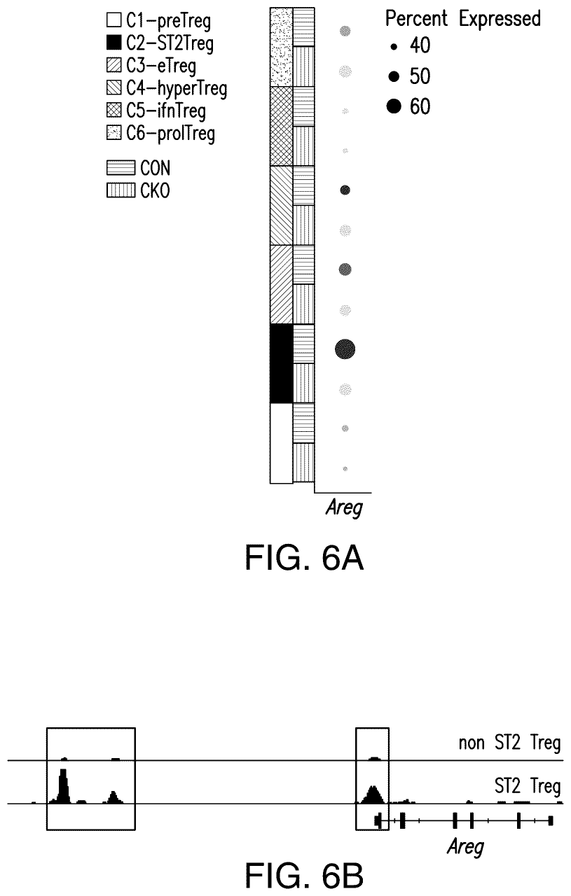

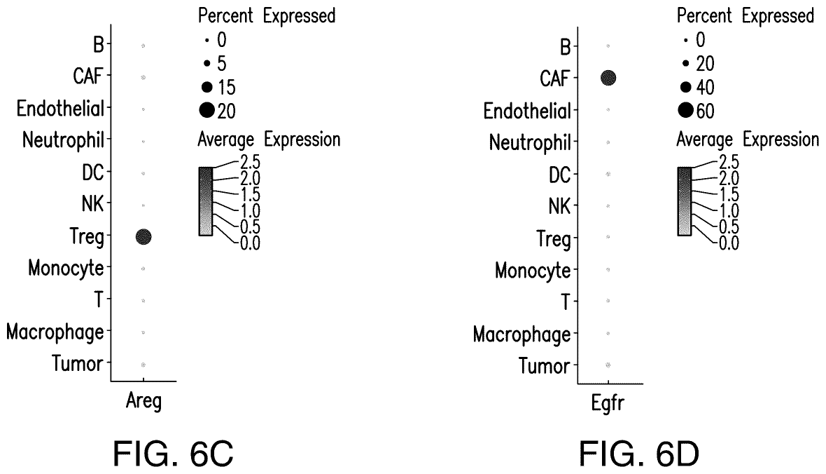

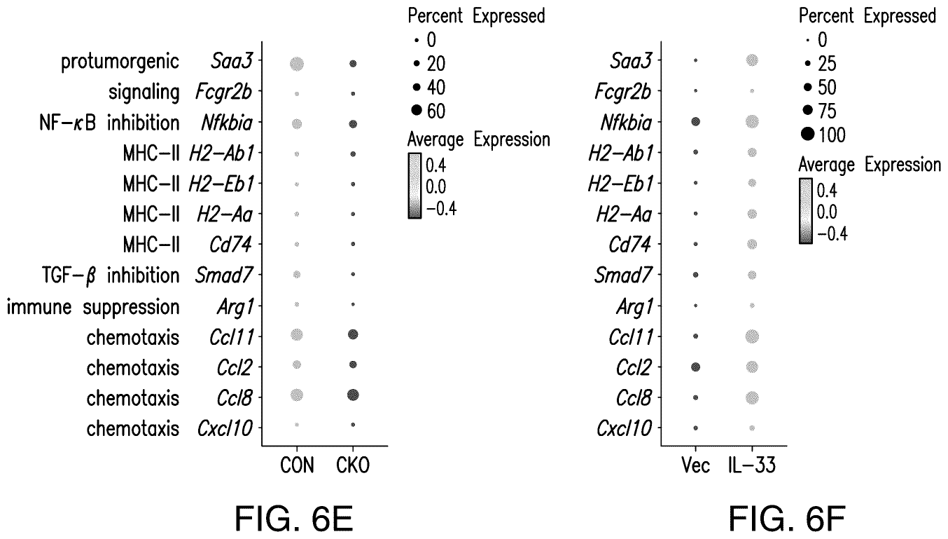

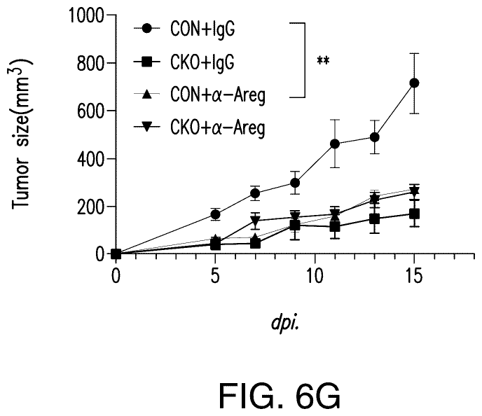

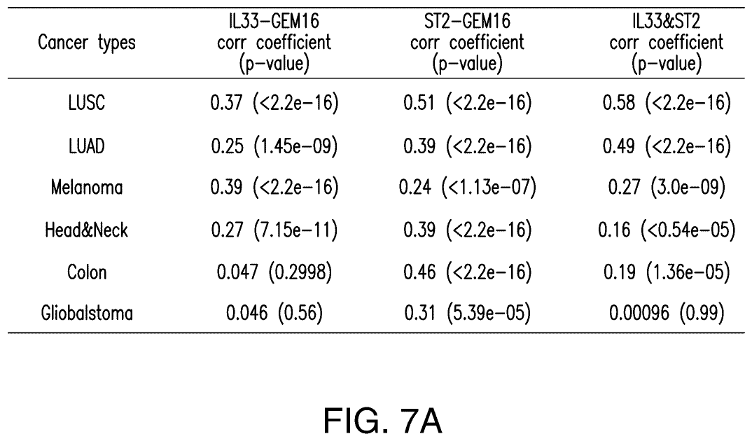

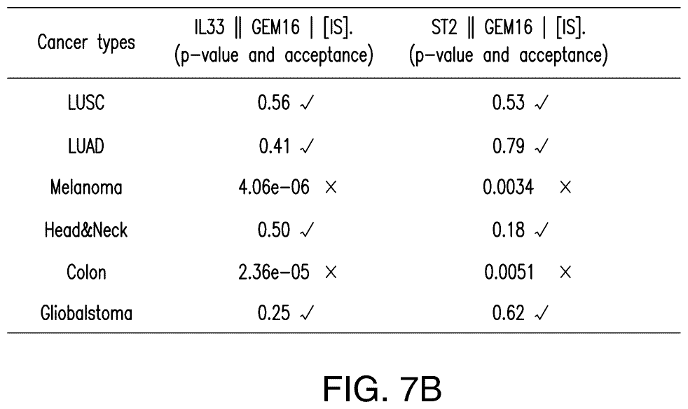

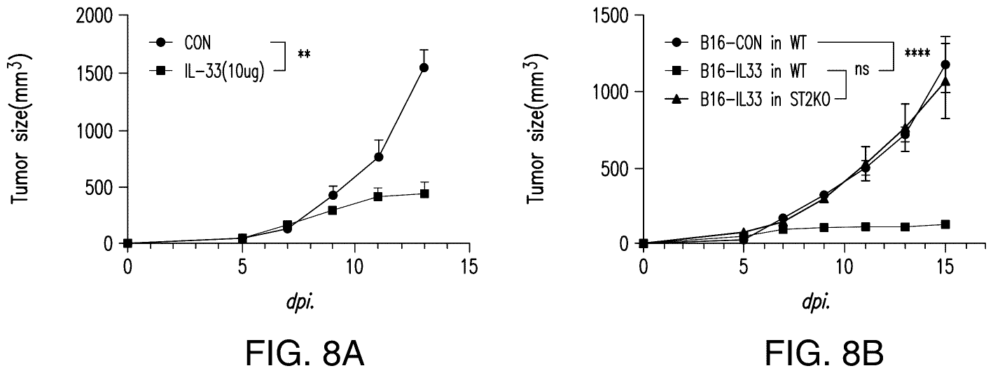

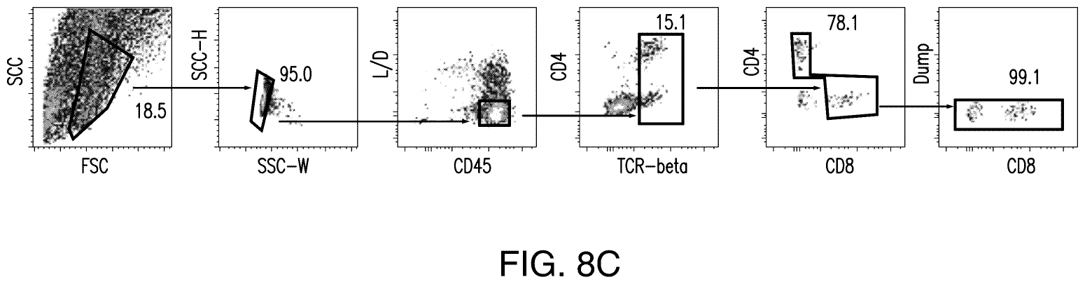

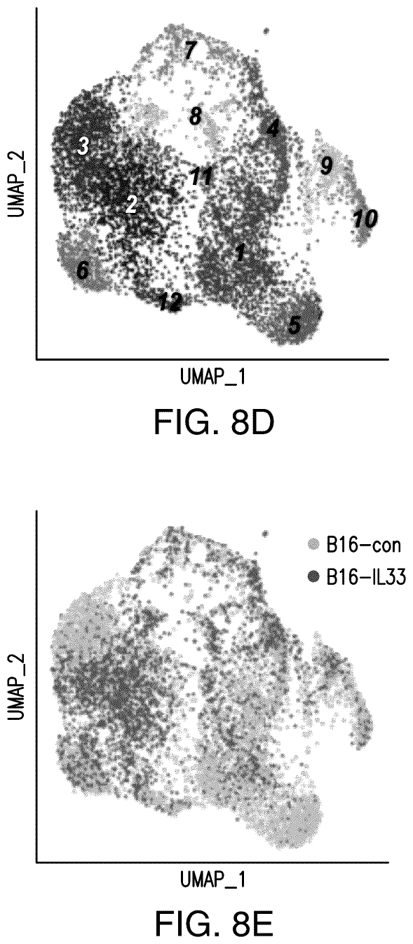

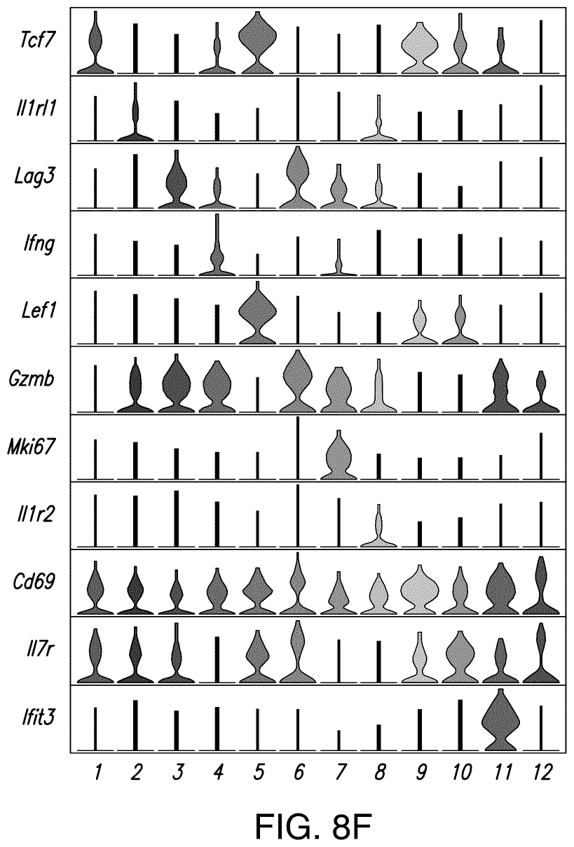

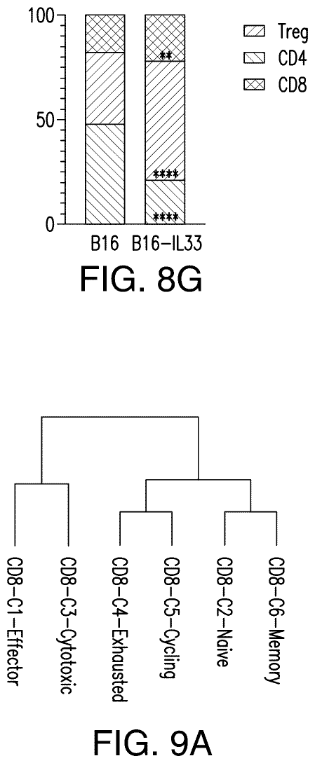

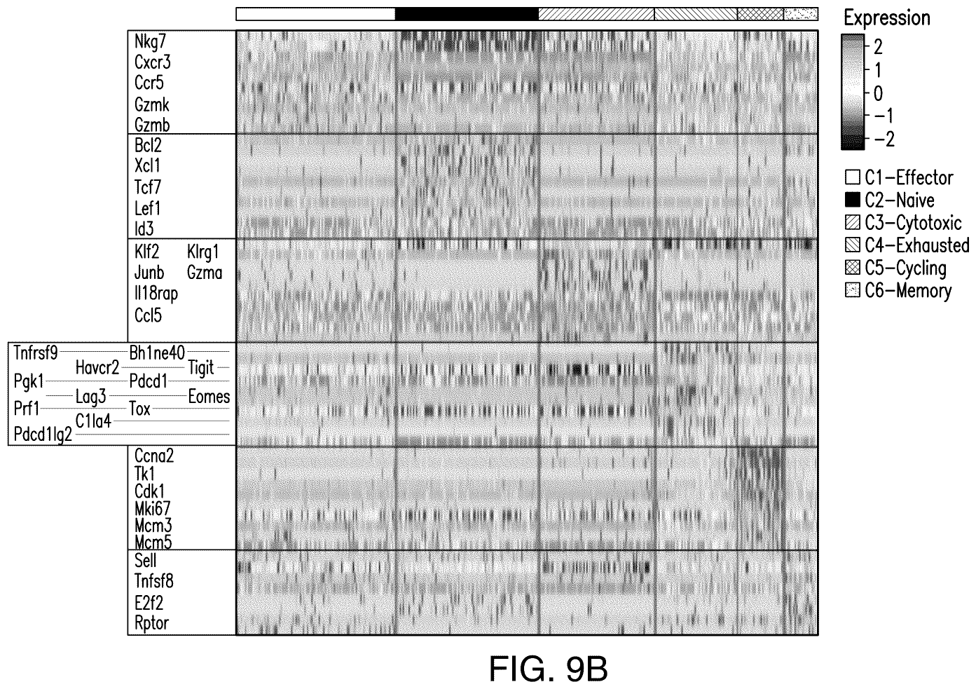

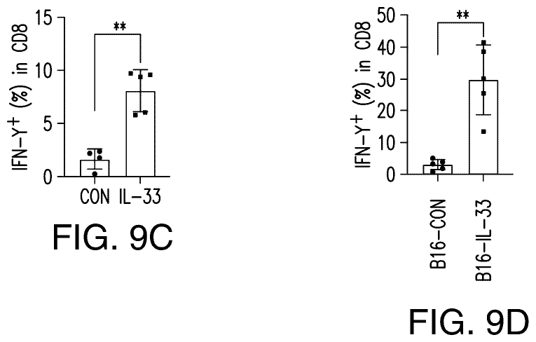

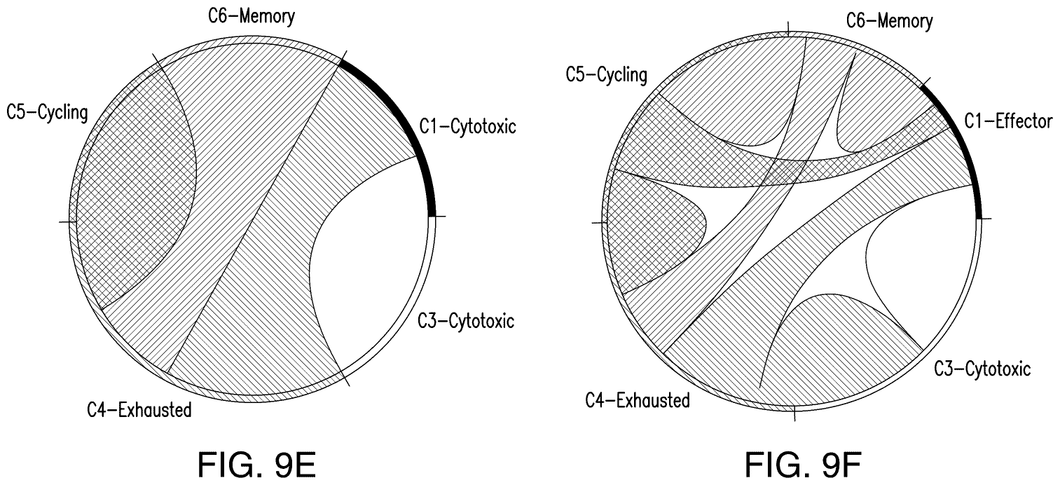

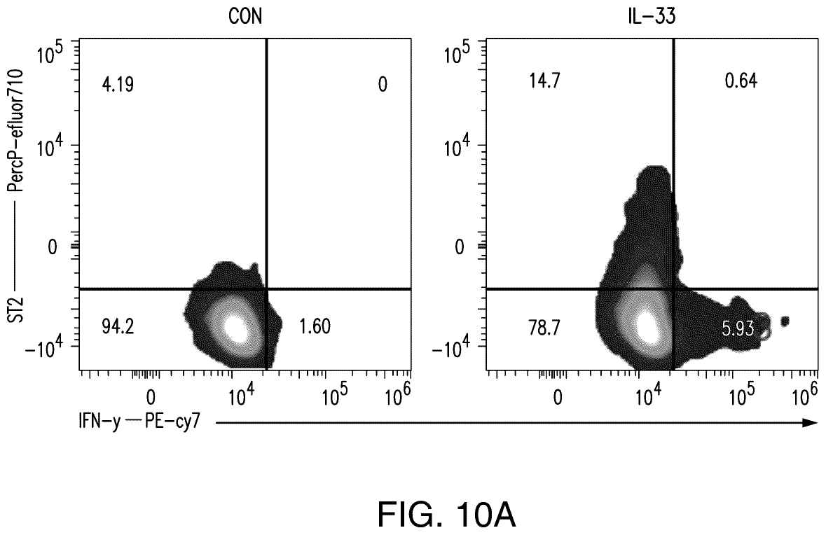

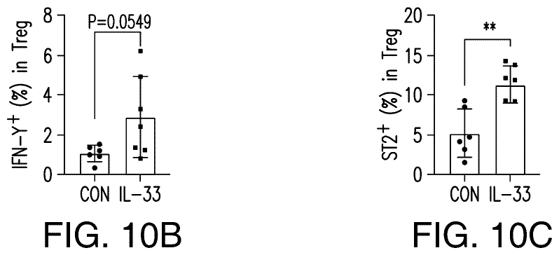

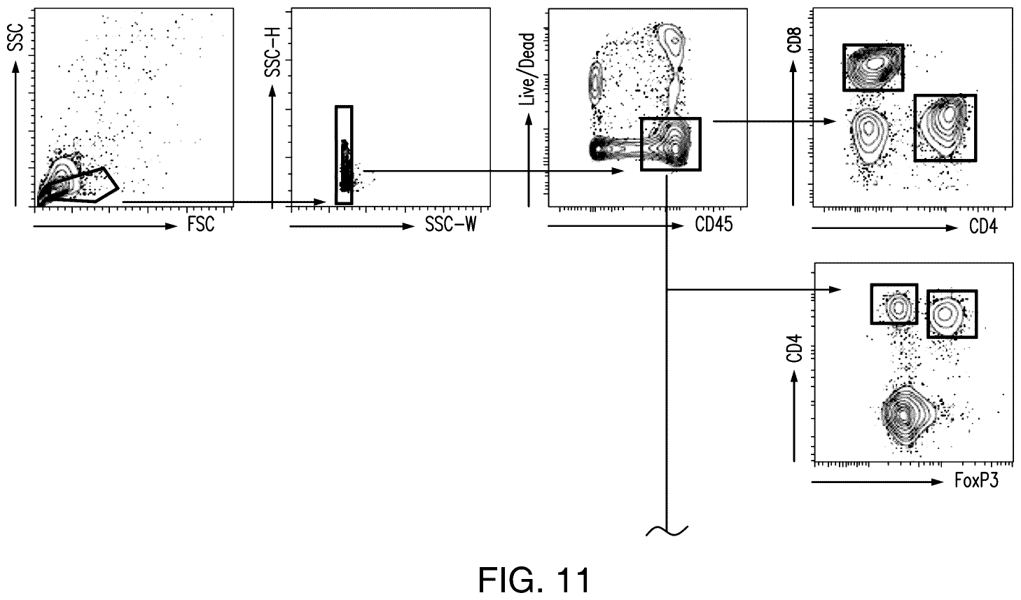

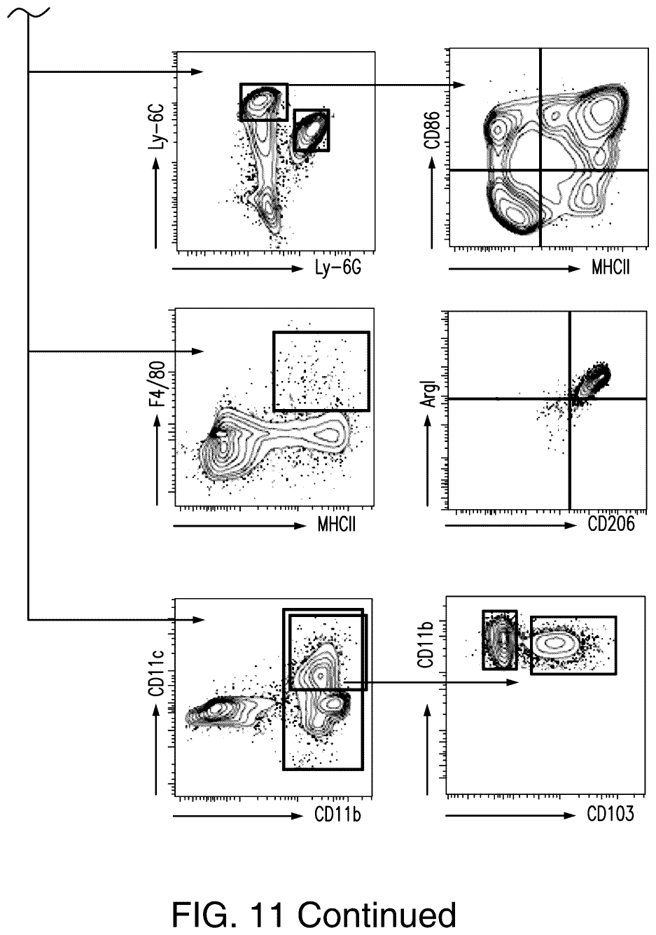

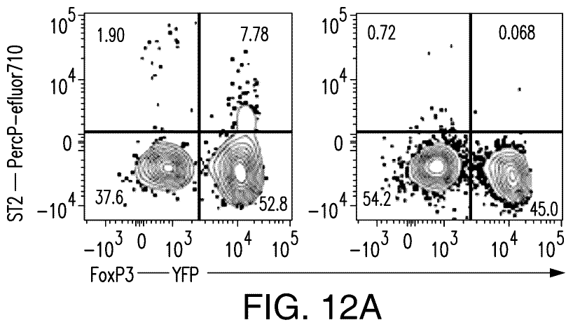

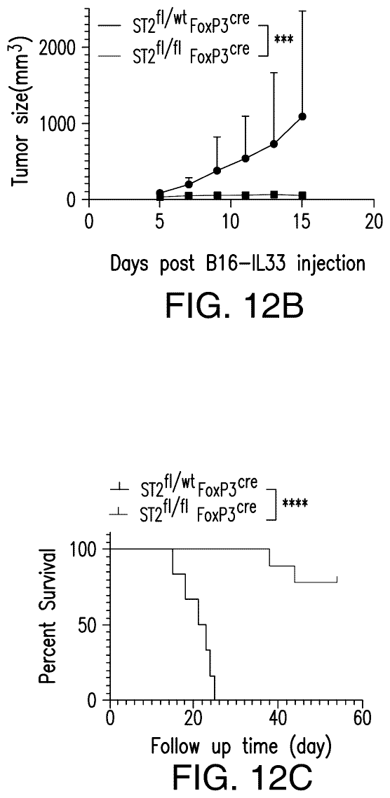

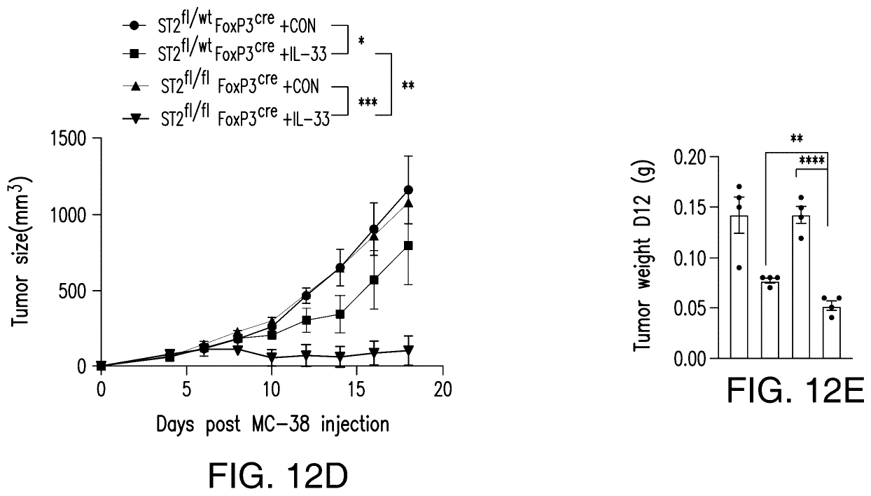

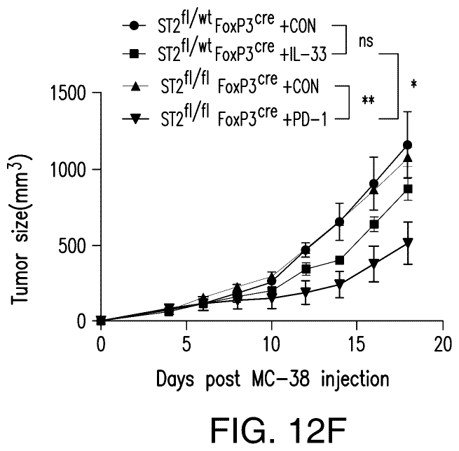

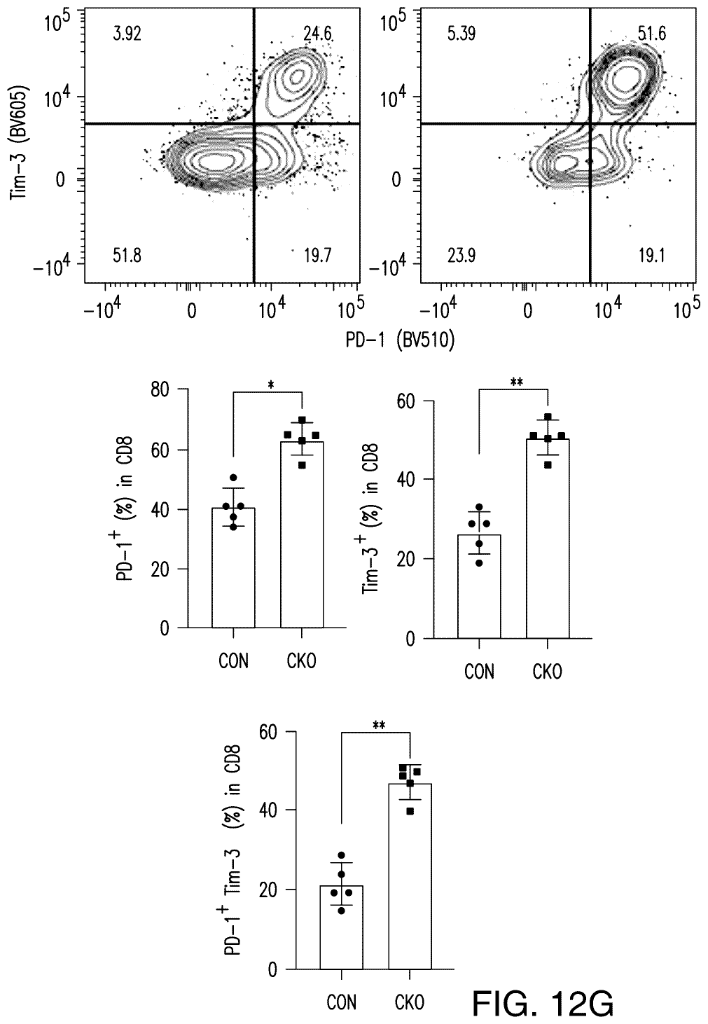

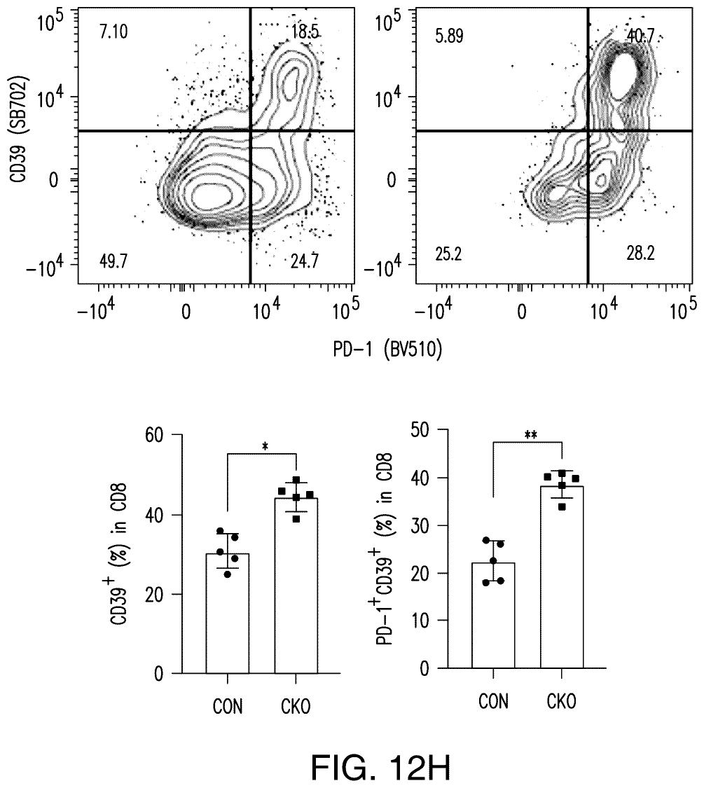

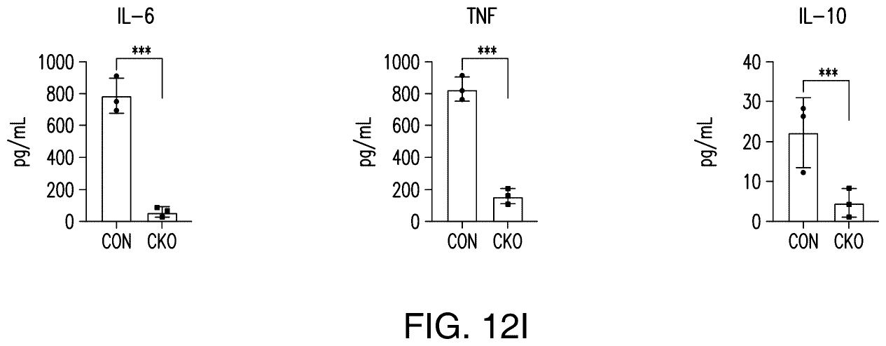

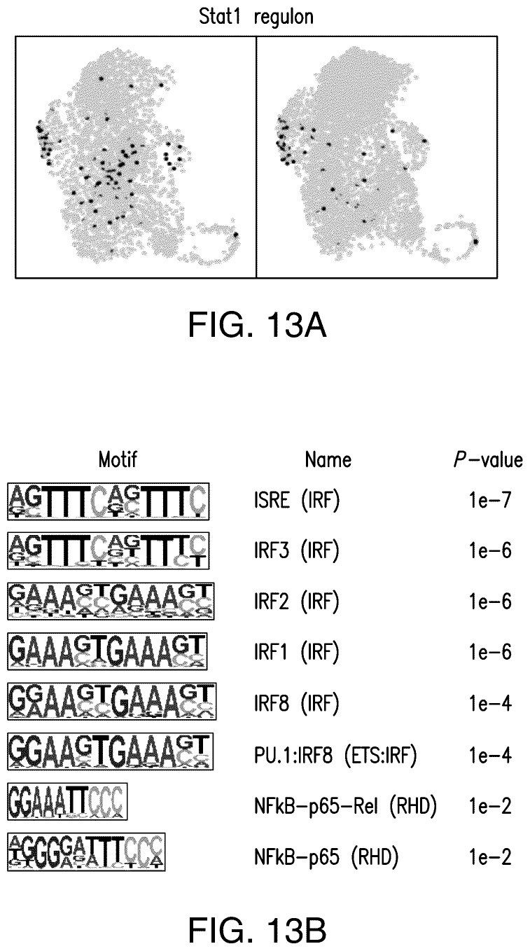

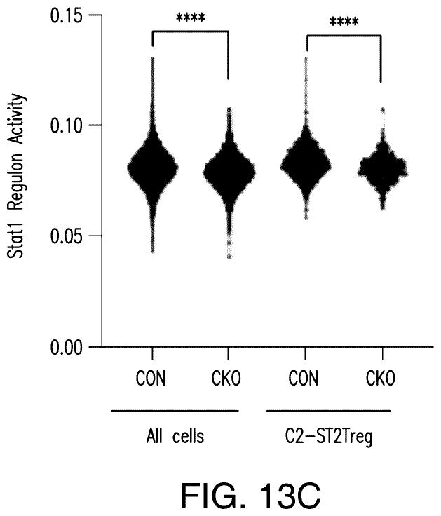

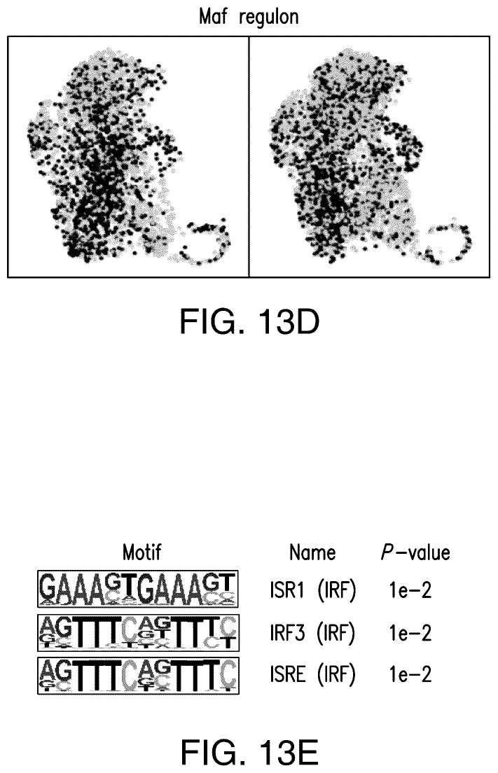

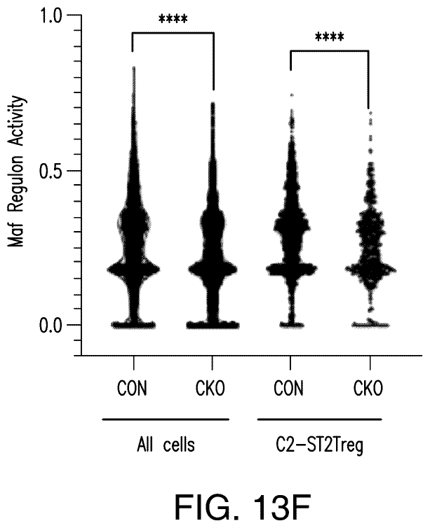

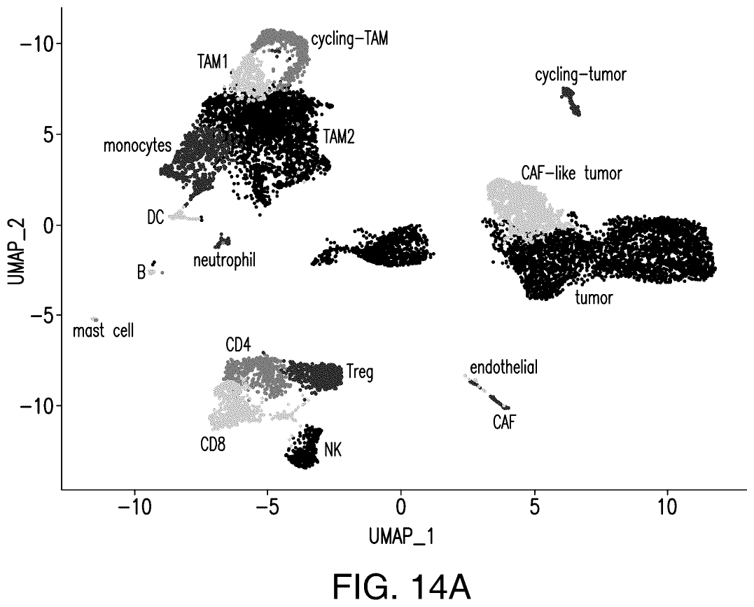

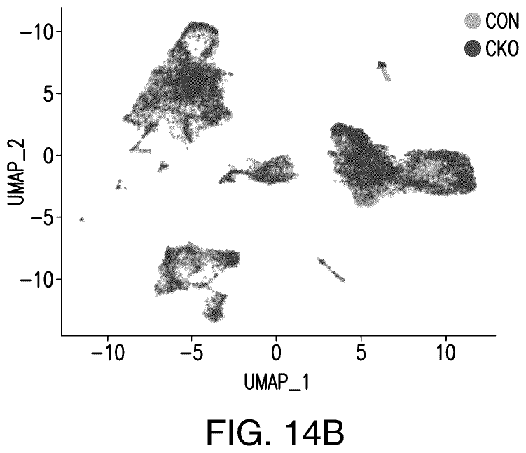

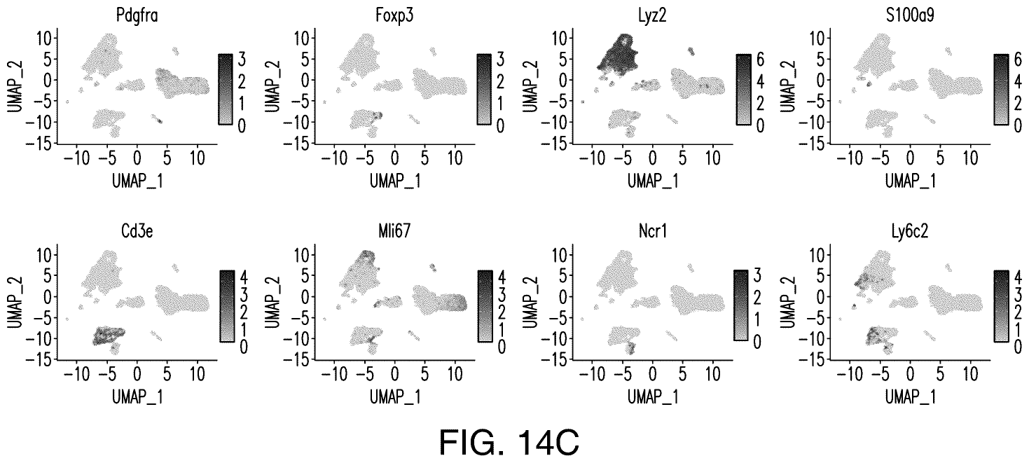

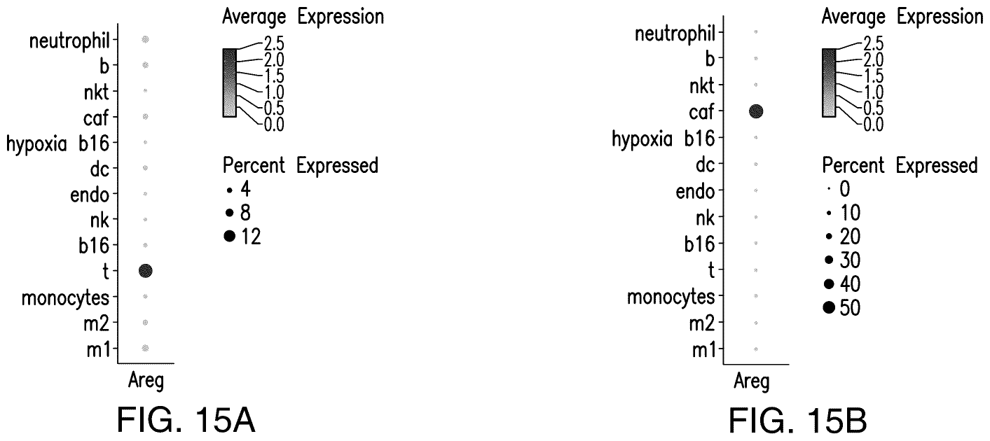

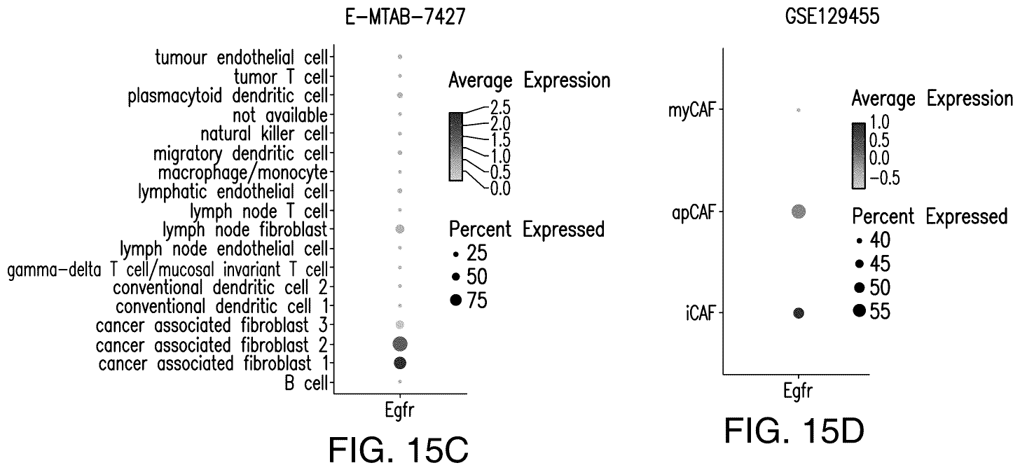

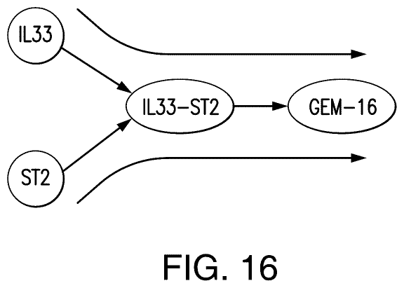

Treg cells. FIG. 3E shows a representative flow cytometry plot and quantitative plot showing the percentage of Tcf1⁺ Treg cells. FIG. 3F shows a representative flow cytometry plot and quantitative plot of the percentage of PD-1⁺, Tim-3⁺, or PD-1⁺Tim-3⁺ Treg cells. FIG. 3G shows a representative flow cytometry plot and quantitative plot showing the percentage and number of CD8⁺ T cells. FIG. 3H shows a representative flow cytometry plot and quantitative plot showing the percentage of Tcf1⁺ CD8⁺ T cells. FIG. 3I shows a representative flow cytometry plot and quantitative plot showing the percentage of Ki-67⁺ CD8⁺ T cells. FIG. 3J shows a representative flow cytometry plot and quantitative plot showing the percentage of GzmB+CD8⁺ T cells and IFN-γ⁺ CD8⁺ T cells. FIG. 3K shows a representative flow cytometry plot and quantitative plot showing the percentage of ILC2 cells.

FIGS. 4A-4G show that specific deletion of on Il1rl1 in Tregs altered myeloid cell population in the TME. FIG. 4A shows a representative flow cytometry plot and quantitative plot showing the percentage CD11b⁺ cells in total CD45⁺ cells. FIG. 4B shows a representative flow cytometry plot and quantitative plot showing the percentage and number of monocytic MDSCs and granulocytic MDSCs. FIG. 4C shows a representative flow cytometry plot and quantitative plot of the percentage of CD86+, MHCII+, or CD86+MHCII +monocytic MDSCs. FIG. 4D shows a representative flow cytometry plot and quantitative plot showing the percentage of dendritic cells in total CD45⁺ cells. FIG. 4E shows a representative flow cytometry plot and quantitative plot showing the percentage of CD103⁺ dendritic cells. FIG. 4F shows a representative flow cytometry plot and quantitative plot showing the percentage and number of infiltrating macrophages. FIG. 4G shows a representative flow cytometry plot and quantitative plot showing the percentage and number of typel and type2 macrophages.

FIGS. 5A-5J show that global transcriptional landscape of Treg cells from tumor bearing CON and CKO mice. FIG. 5A shows UMAP dimensionality reduction projects of Treg cells from B16-IL33 tumor bearing CON(Foxp3^{cre}) and CKO (Foxp3^{creIl}1r1^{flox/flox}) mice to 2 dimensions showing 6 subclusters differentiated by color and trajectory analysis of different CD8 clusters. Each point represents a single cell, with cells of similar gene expression profiles positioned closer together in the projection. FIG. 5B shows UMAP projections comparing the distribution of Treg cells in CON(Foxp3^{cre}) and CKO (Foxp3^{creIl}1r1^{flox/flox}) mice. FIG. 5C depicts a bar plot showing the percentage of cells in each Treg cell cluster based on tumor-origin (CON(Foxp3^{cre}) mice vs CKO (Foxp3^{creIl}1r1^{flox/flox}) mice). FIG. 5D depicts a bar plot showing the percentage of clonally expanded cells in each Treg cell cluster. FIG. 5E shows a UMAP projection comparing the distribution of Bcl3 regulon in B16-CON and B16-IL33 tumors. FIG. 5F shows motifs enriched in the promoter region of Bcl3 target genes. FIG. 5G depicts a violin plot showing the Bcl3 regulon activity of Treg cells in all clusters and ST2Treg cluster. FIG. 5H shows a UMAP projection comparing the distribution of Nfkb2 regulon in B16-CON and B16-IL33 tumors. FIG. 5I shows motifs enriched in the promoter region of Nfkb2 target genes. FIG. 5J depicts a violin plot showing the Nfkb2 regulon activity of Treg cells in all clusters and ST2Treg cluster.

FIGS. 6A-6G show that AREG/EGFR enabled IL1RL1⁺ Tregs and CAFs crosstalk drove tumor immune suppression. FIG. 6A depicts a dot-plot showing the AREG gene expression across all Treg clusters in CON(Foxp3ce) and CKO (Foxp3^{creIl}1r1^{flox/flox}) mice. FIG. 6B shows a genome track of AREG locus in lung tissue, ST2 Treg cells, and other Treg cells. FIG. 6C depicts a dot-plot showing the AREG gene expression level across all clusters in MC38 tumor microenvironment. FIG. 6D depicts a dot-plot showing the EGFR gene expression level across all clusters in MC38 tumor microenvironment. FIG. 6E depicts a dot-blot showing differential expressed genes between CON(Foxp3^{cre}) and CKO (Foxp3^{creIl}1r1^{flox/flox}) mice. FIG. 6F depicts a dot-blot showing differential expressed genes in response to IL-33 treatment. B16-IL33 tumor cells (1x105) were inoculated i.d. into the right flank of the CON(Foxp3^{cre}) and CKO Foxp3^{creIl}1r1^{flox/flox})) mice. FIG. 6G shows tumor growth over time in CON(Foxp3^{cre}) and CKO (Foxp3^{creIl}1r1^{flox/flox})) mice with and without treatment with an anti-AREG antibody. Anti-AREG antibody (50 ug/mouse) was treated starting from d5, and every four days for a total of 3 times.

FIGS. 7A and 7B show gene expression module (GEM) regulated by ST2 signaling. GEM16 represents a gene signature of a subpopulation of Tregs, whose expression is regulated by IL33/ST2 signaling. FIG. 7A depicts a table showing that the expression of IL33 and GEM16 were significantly correlated in multiple cancer types except in COAD and GBM. FIG. 7B depicts a table showing that the expression of ST2 and GEM16 were significantly correlated in multiple cancer types.

FIGS. 8A-8G show the global transcriptional landscape of T cells from B16-CON and B16-IL33. FIG. 8A shows B16 tumor size over time in mice treated with IL-33. B16 tumor cells (1×10⁵) were inoculated i.d. into the right flank of the C57BL/6J mice, and mice were treated with IL-33 protein or PBS on day 5 and again every 4 days for a total of 3 times. Tumor sizes were monitored every 2 days, and average tumor sizes are shown. FIG. 8B shows tumor growth in mice inoculated i.d. with either B16-CON tumor cells or B16-IL33 tumor cells. Cells (1×10⁵) were inoculated i.d. into the right flank of the C57BL/6J mice or ST2 knock out mice. Tumor sizes were monitored every 2 days, and average tumor sizes are shown. FIG. 8C shows a gating strategy to cover T cells from B16-CON and B16-IL33 tumors for single cell RNA sequencing. FIG. 8D shows UMAP dimensionality reduction projects for T cells from B16 and B16-IL33 tumors to 2 dimensions showing 12 subclusters differentiated by color. Each point represents a single cell, with cells of similar gene expression profiles positioned closer together in the projection. FIG. 8E shows a UMAP projection comparing the distribution of T cells in B16-CON and B16-IL33 tumors. FIG. 8F shows unsupervised clustering identified 12 clusters based on expression profiles. A violin plot shows the expression of the top marker gene for all 12 clusters based on adjusted P-value and log2FC. FIG. 8G depicts a bar plot showing the percent distribution of these cell types in B16 T cells compared with B16-IL33 T cells. * p<0.05; ** p<0.01; *** p<0.001; **** p<0.0001, Exact one-proportion z test.

FIGS. 9A-9F shows sub-cluster populations of CD CD8⁺ T cells. FIG. 9A shows hierarchical clustering of all six CD8⁺ T cell clusters based on average gene expression. FIG. 9B shows a heatmap of differentially expressed genes of each CD8⁺ T cell cluster. FIG. 9C depicts a bar plot showing the percentage of IFN-γ⁺ CD8 T cells in PBS or IL-33 treated MC38 tumor bearing mice. FIG. 9D depicts a bar plot showing the percentage of IFN-γ⁺ CD8 T cells in B16-CON and B16-IL-33 tumor bearing mice. FIG. 9E shows chord plot showing the detailed TCR sharing situations across six CD8⁺ T cell clusters in B16-CON. FIG. 9F shows chord plot showing the detailed TCR sharing situations across six CD8⁺ T cell clusters in B16-IL33.

FIGS. 10A and 10B show that ST2+ Treg cells were induced after IL-33 treatment. FIG. 10A shows representative flow cytometry plot showing ST2 and IFN-γ staining gated on Treg cells. MC38 tumor cells (1×10⁶) were inoculated i.d. into the right flank of the C57BL/6J mice, IL-33 protein and PBS treatments started from day 5 and again every 4 days for a total of 3 times. FIG. 10B depicts a bar plot showing the percentage of ST2+or IFN-γ⁺ Treg cells.

FIG. 11 shows the flow cytometry gating strategy for identifying tumor-infiltrating lymphocytes and myeloid cells, as disclosed in FIG. 3 and FIG. 4.

FIGS. 12A-12I show that specific deletion of ILIRL in Tregs altered the TME. FIG. 12A shows a representative flow cytometry plot showing the ST2 staining in Treg cells. FIGS. 12B shows tumor growth in mice inoculated i.d. with B16-IL33 tumor cells. B16-IL33 tumor cells (1×10⁵) were inoculated i.d. into the right flank of the CON(Foxp3^{cre}) and CKO (Foxp3^{creIl}1r1^{flox/flox}) mice. Tumor size was monitored every two days. FIG. 12C shows the overall survival of B16-IL33 tumor bearing mice. FIG. 12D shows the tumor curve of MC38 tumor bearing CON(Foxp3^{cre}) or CKO (Foxp3^{creIl}1r1^{flox/flox}) mice treated with IL-33. FIG. 12E shows the tumor weight of MC38 tumor bearing mice on dayl2 after inoculation. FIG. 12F shows tumor growth of MC38 tumor bearing CON(Foxp3^{cre}) or CKO (Foxp3^{creIl}1r1^{flox/flox}) mice treated with anti-PD-1 antibody. FIG. 12G shows a representative flow cytometry plot and quantitative plot of the percentage of PD-1⁺, Tim-3⁺, or PD-1⁺Tim-3⁺ T cells. FIG. 12H shows a representative flow cytometry plot and quantitative plot of the percentage of 39+or PD-1⁺CD39⁺ CD8⁺ T cells. FIG. 12I illustrates a bar plot showing the IL-6, TNF, and IL-10 level in tumor extracts from CON(Foxp3^{cre}) or CKO (Foxp3^{creIl}1r1^{flox/flox}) mice.

FIGS. 13A-13F show that Stat 1 regulon upregulated in IL1RL1+ Treg cells. FIG. 13A shows a UMAP projection comparing the distribution of Stat 1 regulon in B16-CON and B16-IL33 tumors. FIG. 13B illustrates motif enriched in the promoter region of Stat1 target genes. FIG. 13C shows a violin plot showing the Stat1 regulon activity of Treg cells in all clusters and ST2Treg cluster. FIG. 13D shows a UMAP projection comparing the distribution of Maf regulon in B16-CON and B16-IL33 tumors. FIG. 13E illustrates motif enriched in the promoter region of Maf target genes. FIG. 13F depicts a violin plot showing the Maf regulon activity of Treg cells in all clusters and ST2Treg cluster.

FIGS. 14A-14C show UMAP projects comparing gene clusters in MC38 tumors treated with IL-33. FIG. 14A shows a UMAP dimensionality reduction projects of all cells from PBS or IL-33 treated MC38 tumors to 2 dimensions showing different subclusters differentiated by color. Each point represents a single cell, with cells of similar gene expression profiles positioned closer together in the projection. FIG. 14B shows a UMAP projection comparing the distribution of cells from PBS or IL-33 treated MC38. FIG. 14C shows a UMAP projection of several cluster specific genes.

FIGS. 15A-15D illustrate EGFR expression pattern in mouse melanoma tumors and patients with pancreatic ductal adenocarcinoma (PDAC). FIG. 15A depicts a dot-plot showing the AREG gene expression level across all clusters in B16-IL33 tumor microenvironment. FIG. 15B depicts a dot-plot showing the EGFR gene expression level across all clusters in B16-IL33 tumor microenvironment. FIG. 15C depicts a dot-plot showing the EGFR gene expression level across all clusters in B16 tumor microenvironment. FIG. 15D shows data reanalyzed from E-MTAB-7427 represented in a dot-plot showing the EGFR gene expression level across all CAF clusters in PDAC cancer microenvironment. Data were reanalyzed from GSE129455.

FIG. 16 shows a representative scheme illustrating the conditional independence tested herein. The biological knowledge regarding IL33, ST2, and GEM16 can be represented as the causal graph, in which each node represents a variable and directed edges represent the causal relationship. It was assumed that there is no latent variable that causally regulated the nodes in the graph. Two conditional independence tests were performed, including nodes along the paths from IL33 and ST2 to GEM16.

### DETAILED DESCRIPTION OF THE INVENTION

Immune checkpoint blockade (ICB) cancer therapy has considerably prolonged the overall survival of cancer patients. It is a pressing goal, however, in cancer immunotherapy to increase the ICB response rate and the long-term survival. Interleukin-33 (IL-33), a member of the IL-1 cytokine family and derived from epithelial cells, has been shown to mediate the antitumor effect of immune checkpoint inhibitors and also displays strong antitumor activities when administered as a single agent. However, IL-33 also induces cellular responses in the tumor microenvironment (TME) that constrain its antitumor efficacy. The present disclosure is based, in part, on the demonstration that IL-33 induced strong CD8⁺ T cell antitumor immune responses characterized by robust clonal expansion and functional diversification. Nonetheless, the present disclosure shows that IL-33 induced intense regulatory T cells (Treg) accumulation in the TME, which was dominated by the IL1RL1+ Treg subset. The present disclosure further shows that the IL1RL1 signaling in Treg cells greatly dampened the antitumor activity of IL-33. Whole tumor single cell single-cell RNA sequencing (scRNA-seq) analysis revealed that the amphiregulin (AREG)-EGFR axis mediated crosstalk between Treg and carcinoma carcinoma-associated fibroblasts (CAF). The present disclosure demonstrates that an anti-AREG antibody and IL-33 synergistically inhibited tumor growth and establishes that the AREG/EGFR axis mediates Treg/CAF coupling, posing a key barrier for cancer immunotherapy.

Non-limiting embodiments of the present disclosure are described by the present specification and Examples.

For purposes of clarity of disclosure and not by way of limitation, the detailed description is divided into the following subsections:
1. Definitions;
2. Interleukin 33;
3. Anti-Amphiregulin Antibodies;
4. Pharmaceutical Compositions and Methods of Treatment; and
5. Kits.

### 1. Definitions

Unless defined otherwise, all technical and scientific terms used herein have the meaning commonly understood by a person skilled in the art to which this disclosed subject matter belongs. The following references provide one of skill with a general definition of many of the terms used in this disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of,” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

An “individual” or “subject” herein is a vertebrate, such as a human or non-human animal, for example, a mammal. Mammals include, but are not limited to, humans, non-human primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” or “therapeutically effective amount” is an amount effective, at dosages and for periods of time necessary, that produces a desired effect, e.g., the desired therapeutic or prophylactic result. In certain embodiments, an effective amount can be formulated and/or administered in a single dose. In certain embodiments, an effective amount can be formulated and/or administered in a plurality of doses, for example, as part of a dosing regimen.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing cancer, decreasing the rate of disease progression, amelioration or palliation of the disease state, and remission or improved prognosis. By “preventing” progression of a disease or disorder, a treatment can prevent deterioration due to a disorder (e.g., a cancer) in an affected or diagnosed subject or a subject suspected of having the disorder, but also a treatment can prevent the onset of the disorder or a symptom of the disorder in a subject at risk for the disorder or suspected of having the disorder.

“In combination with,” as used herein, means that the presently disclosed combination of IL33 polypeptide and anti-AREG antibody is administered with one or more agents, e.g., an immunomodulatory agent, to a subject as part of a treatment regimen or plan.

As used herein, the term “anti-cancer effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse, or progression-free survival in a subject diagnosed with cancer

By “increase” is meant to alter positively by at least about 5%. A positive alteration can be an increase of about 5%, about 10%, about 25%, about 30%, about 50%, about 75%, about 100% or more.

By “reduce” is meant to alter negatively by at least about 5%. A negative alteration can be a decrease of about 5%, about 10%, about 25%, about 30%, about 50%, about 75% or more, even by about 100%.

The terms “nucleic acid sequence” and “polynucleotide,” as used herein, refer to a single or double-stranded covalently-linked sequence of nucleotides in which the 3′ and 5′ ends on each nucleotide are joined by phosphodiester bonds. The polynucleotide can include deoxyribonucleotide bases or ribonucleotide bases, and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, the term “mutation” refers to a mutation in an amino acid sequence or in a nucleic acid sequence. In certain embodiments, a mutation in an amino acid sequence can be a substitution (replacement), an insertion (addition), or a deletion (truncation) of at least one amino acid in the amino acid sequence. In certain embodiments, a mutation in a nucleic acid sequence can be a substitution (replacement), an insertion (addition), or a deletion (truncation) of at least nucleotide of the nucleic acid sequence.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

As used herein, the terms “antibody” and “antigen-binding fragment” refer to a polypeptide comprising at least a light chain or heavy chain immunoglobulin variable region which specifically recognizes and specifically binds an epitope of an antigen (e.g., amphiregulin) or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies include intact immunoglobulins and variants thereof. Functional fragments (antigen-binding fragments) of antibodies, that specifically bind an antigen (e.g., amphiregulin) are well known in the art, such as Fab fragments, Fab′ fragments, F(ab)′₂ fragments, single chain Fv proteins (“scFv”), and disulfide stabilized Fv proteins (“dsFv”) that specifically bind the target antigen. A scFv protein is a fusion protein in which a light chain variable region of an immunoglobulin and a heavy chain variable region of an immunoglobulin are bound by a linker. In dsFvs, the chains have been mutated to introduce a disulfide bond to stabilize the association of the chains. In certain embodiments, the term also includes genetically engineered forms such as chimeric antibodies (for example, humanized murine antibodies), heteroconjugate antibodies (such as, bispecific antibodies).

A naturally occurring immunoglobulin has heavy (H) chains and light (L) chains interconnected by disulfide bonds. There are two types of light chain, lambda (λ) and kappa (κ). There are five main heavy chain classes (or isotypes) that determine the functional activity of an antibody molecule: IgM, IgD, IgG, IgA and IgE. Each heavy and light chain contains a constant region and a variable region. Light and heavy chain variable regions contain four (4) regions (e.g., FR1, FR2, FR3, and FR4) interrupted by three hypervariable regions, also called “complementarity-determining regions” or “CDR.” The extent of the framework region and CDRs have been defined by designation systems known in the art such as Kabat, Clothia, IMGT, etc. The CDRs are primarily responsible for binding to an epitope of an antigen.

As used herein, the term “monoclonal antibody” refers to an antibody produced by a single clone of B-lymphocytes or by a cell into which the light and heavy chain genes of a single antibody have been transfected. In certain non-limiting embodiments, monoclonal antibodies are produced by making hybrid antibody-forming cells from a fusion of myeloma cells with immune spleen cells. Monoclonal antibodies include humanized monoclonal antibodies.

As used herein, the term “chimeric antibody” refers to an antibody that has framework residues from one species, such as human, and CDRs (which generally confer antigen binding) from another species.

As used herein, the term “human antibody” refers to an antibody that includes human framework regions and all of the CDRs from a human immunoglobulin. In certain embodiments, the framework and the CDRs are from the same originating human heavy and/or light chain amino acid sequence.

As used herein, the term “humanized antibody” refers to an antibody including a human framework region and one or more CDRs from a non-human (e.g., a mouse CDR) antibody. In certain embodiments, a humanized antibody is an antibody comprising a humanized light chain and a humanized heavy chain immunoglobulin. A humanized antibody binds to the same antigen as a donor antibody that provides the CDRs. The acceptor framework of a humanized immunoglobulin or antibody can have a limited number of substitutions by amino acids taken from the donor framework.

As used herein, the terms “epitope” and “antigenic determinant” refer to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least three, and more usually, at least five or eight to ten amino acids in a unique spatial conformation.

As used herein, the term “binding affinity” refers to the affinity of an antibody for an antigen. In certain embodiments, affinity is calculated by a modification of the Scatchard method described by Frankel et al., Mol. Immunol., 16:101-106, 1979. In certain embodiments, binding affinity is measured by an antigen/antibody dissociation rate. In certain embodiments, a high binding affinity is measured by a competition radioimmunoassay. In certain embodiments, binding affinity is measured by ELISA.

As used herein, the term “isolated” refers to any biological molecules (e.g., peptide, antibody) that have been substantially separated or purified away from other biological components in the environment (such as a cell) in which the component naturally occurs, i.e., other proteins or cellular components.

The term “dosage” is intended to encompass a formulation expressed in terms of total amounts for a given timeframe, for example, as μg/kg/hr, μg/kg/day, mg/kg/day, or mg/kg/hr. The dosage is the amount of an ingredient administered in accordance with a particular dosage regimen. A “dose” is an amount of an agent administered to a mammal in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning, in any case, will be apparent from the context.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. Ranges disclosed herein, for example, “between about X and about Y” are, unless specified otherwise, inclusive of range limits about X and about Y as well as X and Y. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50 to 30, 50 to 20, and 50 to 10 in the other direction.

The term “endogenous,” as used herein, refers to a nucleic acid molecule or polypeptide that is normally expressed in a cell or tissuc.

The term “exogenous,” as used herein, refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid can vary from an endogenous counterpart by sequence, by position/location, or both. For clarity, an exogenous nucleic acid can have the same or different sequence relative to its native endogenous counterpart; it can be introduced by genetic engineering into the cell itself or a progenitor thereof, and can optionally be linked to alternative control sequences, such as a non-native promoter or secretory sequence.

### 2. Interleukin 33 (IL33)

The present disclosure provides interleukin 33 for use in the methods disclosed herein. Interleukin 33 (IL33) is a member of the ILI family of cytokines involved in innate and adaptive immune responses via interaction with its receptor, ST2. Activation of ST2 signaling by IL33 triggers pleiotropic immune functions in multiple ST2-expressing immune cells, including macrophages, neutrophils, cosinophils, basophils, mast cells, type 2 helper T cells, regulatory T cells, and group 2 innate lymphoid cells. IL33-mediated effector functions contribute to the tissuc inflammatory and reparative responses in various organs including lung, skin, kidney, central nerve system, cardiovascular system, and gastrointestinal system. Endogenous IL33/ST2 signaling exhibits diverse immune regulatory functions during the progression of different diseases. Modulation of the IL-33/ST2 axis, therefore, represents a promising strategy for treating immune disorders that involve dysregulation of cytokine signaling (e.g., cancer).

IL-33 is expressed in normal epithelial cells of lining tissues like lung and skin but drastically downregulated in high-grade tumor. Tumor-derived IL-33 is crucial for the antitumor efficacy of checkpoint inhibitors. Thus, downregulation of IL-33 is an important mechanism by which tumors evade the immune system. Ample evidence also supports IL-33 as viable cancer immunotherapy. Both tumoral expression and injection of IL-33 inhibit tumor growth through activating type 1 immune responses. In addition, IL-33 can be combined with immune checkpoint inhibitors to produce additive antitumor efficacy in multiple clinical models. Despite its potent activities in inducing type 1 antitumor immune responses, IL-33 can stimulate immune regulatory cells particularly Treg cells. In addition, IL-33 also induces fibrosis in many pathological settings such as liver fibrosis, pancreatitis, kidney diseases, and asthma. Interestingly, it has also been demonstrated that IL-33 is produced by CAFs and directly promotes tumorigenesis and metastasis.

In this scenario, IL1RL1+ Treg cells were initially thought to play immune suppressive and anti-inflammatory roles. However, Treg cells are also directly involved in non-immune regulatory roles particularly tissue repair and maintaining barrier tissue integrity. Recent studies have demonstrated that carcinoma-associated fibroblasts (CAFs) are involved in recruiting Treg cells in breast cancer and pancreatic cancer tissues. In addition, IL-33 produced by stromal cells has been involved in crosstalk between IL1RL1+ Treg and mesenchymal cells in the visceral adipose tissue (VAT). Thus, crosstalk between CAFs and Treg cells can be important for shaping tumor immune tolerance in the TME.

In certain embodiments, the present disclosure provides an IL33 polypeptide or a functional fragment thereof. In certain embodiments, the IL33 polypeptide or functional fragment thereof is a human IL33 polypeptide. In certain embodiments, the IL33 polypeptide includes an amino acid sequence that is at least about 80%, at least about 80%, at least about 85%, at least about 90%, at least about 95%, at least about 96%, at least about 97%, at least about 98%, or at least about 99% identical to the amino acid sequence set forth in UniProt database accession No. 095760. In certain embodiments, the IL33 polypeptide includes substitutions (e.g., conservative substitutions), insertions, or deletions relative to the amino acid sequence set forth in UniProt database accession No. 095760, that do not significantly alter the function or activity of the IL33 polypeptide.

In certain embodiments, the IL33 polypeptide comprises an amino acid sequence that is at least about 85%, at least about 90%, at least about 95%, at least about 96%, at least about 97%, at least about 98%, at least about 99% or at least 100% identical to the amino acid sequence set forth in SEQ ID NO: 1, which is provided below. In certain embodiments, the IL33 polypeptide consists of the amino acid sequence set forth in SEQ ID NO: 1, which is provided below.

(SEQ ID NO: 1)
MKPKMKYSTNKISTAKWKNTASKALCFKLGKSQQKAKEVCPMYFMKLRS
GLMIKKEACYFRRETTKRPSLKTGRKHKRHLVLAACQQQSTVECFAFGI
SGVQKYTRALHDSSITGISPITEYLASLSTYNDQSITFALEDESYEIYV
EDLKKDEKKDKVLLSYYESQHPSNESGDGVDGKMLMVTLSPTKDFWLHA
NNKEHSVELHKCEKPLPDQAFFVLHNMHSNCVSFECKTDPGVFIGVKDN
HLALIKVDSSENLCTENILFKLSET

Physiologically, IL33 undergoes proteolytic processing by CSTG/cathepsin G, ELANE/neutrophil elastase, and or calpains. This process produces C-terminal peptides that are more active than the unprocessed full-length protein. In certain embodiments, the IL33 polypeptide comprises an amino sequence comprising from amino acid 95 to amino acid 270 of SEQ ID NO: 1. In certain embodiments, the IL33 polypeptide comprises an amino sequence comprising from amino acid 99 to amino acid 270 of SEQ ID NO: 1. In certain embodiments, the IL33 polypeptide comprises an amino sequence comprising from amino acid 109 to amino acid 270 of SEQ ID NO: 1.

In certain embodiments, the IL33 polypeptide comprises an amino acid sequence that is at least about 85%, at least about 90%, at least about 95%, at least about 96%, at least about 97%, at least about 98%, at least about 99% or at least 100% identical to the amino acid sequence set forth in SEQ ID NO: 2, which is provided below. In certain embodiments, the IL33 polypeptide consists of the amino acid sequence set forth in SEQ ID NO: 2, which is provided below.

(SEQ ID NO: 2)
MKPKMKYSTNKISTAKWKNTASKALCFKLGKSQQKAKEVCPMYFMKLRS
GLMIKKEACYFRRETTKRPSLKTGRKHKRHLVLAACQQQSTVECFAFGI
SGVQKYTRALHDSSITDKVLLSYYESQHPSNESGDGVDGKMLMVTLSPT
KDFWLHANNKEHSVELHKCEKPLPDQAFFVLHNMHSNCVSFECKTDPGV
FIGVKDNHLALIKVDSSENLCTENILFKLSET

In certain embodiments, the IL33 polypeptide comprises an amino acid sequence that is at least about 85%, at least about 90%, at least about 95%, at least about 96%, at least about 97%, at least about 98%, at least about 99% or at least 100% identical to the amino acid sequence set forth in SEQ ID NO: 3, which is provided below. In certain embodiments, the IL33 polypeptide consists of the amino acid sequence set forth in SEQ ID NO: 3, which is provided below.

(SEQ ID NO: 3)
MKPKMKYSTNKISTAKWKNTASKALCFKLGKSQQKAKEVCPMYFMKLRS
GLMIKKEACYFRRETTKRPSLKTGISPITEYLASLSTYNDQSITFALED
ESYEIYVEDLKKDEKKDKVLLSYYESQHPSNESGDGVDGKMLMVTLSPT
KDFWLHANNKEHSVELHKCEKPLPDQAFFVLHNMHSNCVSFECKTDPGV
FIGVKDNHLALIKVDSSENLCTENILFKLSET

In certain embodiments, the IL33 polypeptide comprises an amino acid sequence that is at least about 85%, at least about 90%, at least about 95%, at least about 96%, at least about 97%, at least about 98%, at least about 99% or at least 100% identical to the amino acid sequence set forth in SEQ ID NO: 4, which is provided below. In certain embodiments, the IL33 polypeptide consists of the amino acid sequence set forth in SEQ ID NO: 4, which is provided below.

(SEQ ID NO: 4)
MKPKMKYSTNKISTAKWKNTASKALCFKLGNKVLLSYYESQHPSNESGD
GVDGKMLMVTLSPTKDFWLHANNKEHSVELHKCEKPLPDQAFFVLHNMH
SNCVSFECKTDPGVFIGVKDNHLALIKVDSSENLCTENILFKLSET

In certain embodiments, the IL33 polypeptide or functional fragment thereof is a murine IL33 polypeptide. In certain embodiments, the IL33 polypeptide includes an amino acid sequence that is at least about 80%, at least about 80%, at least about 85%, at least about 90%, at least about 95%, at least about 96%, at least about 97%, at least about 98%, or at least about 99% identical to the amino acid sequence set forth in UniProt database accession No. Q8BVZ5. In certain embodiments, the IL33 polypeptide includes substitutions (e.g., conservative substitutions), insertions, or deletions relative to the amino acid sequence set forth in UniProt database accession No. Q8BVZ5, that do not significantly alter the function or activity of the IL33 polypeptide.

In certain embodiments, the IL33 polypeptide comprises an amino acid sequence that is at least about 85%, at least about 90%, at least about 95%, at least about 96%, at least about 97%, at least about 98%, at least about 99% or at least 100% identical to the amino acid sequence set forth in SEQ ID NO: 5, which is provided below. In certain embodiments, the IL33 polypeptide consists of the amino acid sequence set forth in SEQ ID NO: 5, which is provided below.

(SEQ ID NO: 5)
MRPRMKYSNSKISPAKFSSTAGEALVPPCKIRRSQQKTKEFCHVYCMRL
RSGLTIRKETSYFRKEPTKRYSLKSGTKHEENFSAYPRDSRKRSLLGSI
QAFAASVDTLSIQGTSLLTQSPASLSTYNDQSVSFVLENGCYVINVDDS
GKDQEQDQVLLRYYESPCPASQSGDGVDGKKLMVNMSPIKDTDIWLHAN
DKDYSVELQRGDVSPPEQAFFVLHKKSSDFVSFECKNLPGTYIGVKDNQ
LALVEEKDESCNNIMFKLSKI

In certain embodiments, conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. Amino acids can also be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, no more than one, no more than two, no more than three, no more than four, no more than five residues within a specified sequence are altered. Exemplary conservative amino acid substitutions are shown in Table 1 below.

TABLE 1

| Original Residue | Exemplary Conservative Amino Acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gin (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

### 3. Anti-Amphiregulin Antibodies

The present disclosure provides anti-amphiregulin (anti-AREG) antibodies for use in the methods disclosed herein. Amphiregulin (AREG) is a member of the EGF family of cytokines which is comprised of at least ten proteins including EGF, TGF-alpha, HB-EGF, and the various heregulins. All of these cytokines are synthesized as transmembrane precursors and are characterized by the presence of one or several EGF structural units in their extracellular domain. The soluble forms of these cytokines are released by proteolytic cleavage. Amphiregulin was originally isolated from the conditioned media of a PMA-treated MCF-7 human breast carcinoma cell line. The AREG cDNA encodes a 252 amino acid (aa) residue transmembrane precursor. Multiple forms of native AREG containing either 78 or 84 aa residues and both N-and O-linked oligosaccharides have been found. Amphiregulin mRNA expression can be detected in numerous carcinoma cell lines and the epithelial cells of various human tissues including colon, stomach, breast, ovary, kidney, etc. Human AREG stimulates the proliferation of various human and mouse keratinocytes, mammary epithelial cells, and some fibroblasts. The 98 aa residue long-form of recombinant amphiregulin has shown to be approximately 5-10 fold more active than the 78 aa residue form of recombinant AREG.

In certain embodiments, the anti-AREG antibodies specifically bind to a human amphiregulin. In certain embodiments, human AREG comprises the amino acid sequence set forth in SEQ ID NO: 6. SEQ ID NO: 6 is provided below.

(SEQ ID NO: 6)
MRAPLLPPAPVVLSLLILGSGHYAAGLDLNDTYSGKREPFSGDHSADGF
EVTSRSEMSSGSEISPVSEMPSSSEPSSGADYDYSEEYDNEPQIPGYIV
DDSVRVEQVVKPPQNKTESENTSDKPKRKKKGGKNGKNRRNRKKKNPCN
AEFQNFCIHGECKYIEHLEAVTCKCQQEYFGERCGEKSMKTHSMIDSSL
SKIALAAIAAFMSAVILTAVAVITVQLRRQYVRKYEGEAEERKKLRQEN
GNVHAIA

In certain embodiments, the anti-AREG antibody is a monoclonal antibody or a functional fragment thereof. In certain embodiments, the monoclonal antibody is a human antibody. In certain embodiments, the monoclonal antibody is a humanized antibody. In certain embodiments, the monoclonal antibody is a chimeric antibody. In certain embodiments, the anti-AREG antibody is an scFv.

Non-limiting examples of anti-AREG antibodies encompassed by the present disclosure include AF262 (R&D Systems®), AF989 (R&D Systems®), MAB262 (R&D Systems®), MAB989 (R&D Systems®), 16036-1-AP (Invitrogen®), AREG559 (Invitrogen®), PA5-16621 (Invitrogen®), PA5-27298 (Invitrogen®), MA5-41546 (Invitrogen®), MA5-41547 (Invitrogen®), A9 (Invitrogen®), PA5-16616 (Invitrogen®), PA5-110750 (Invitrogen®), PA5-102501 (Invitrogen®), PA5-109404 (Invitrogen®), 1A1G9 (ProteinTech®), 3E4 (Abnova®), and 3847R (Bioss®). Additional examples of anti-AREG antibodies encompassed by the present disclosure are described in U.S. Pat. No. 8,846,868 and U.S. Patent Publication No. 2017/0002068, the contents of each of which are incorporated by reference in their entireties.

In certain embodiments, the anti-AREG antibodies can be produced by methods known in the art or as disclosed herein. In certain embodiments, the anti-AREG antibody can be labeled. For example, and without any limitation, the anti-AREG antibody can be labeled with a radioisotope, fluorescent compound, a chemiluminescent compound, an enzyme, an enzyme co-factor, or any other labels known in the art.

In certain embodiments, the anti-AREG antibody can be monospecific or multispecific (e.g., bispecific, trispecific, or of greater multispecificity). Multispecific antibodies can be specific for different epitopes of a target antigen (e.g., AREG), or can be specific for both AREG and a heterologous epitope, such as a heterologous glycan or peptide. Additional details of multispecific antibodies can be found in Deshaies, Nature 580, 329-338 (2020).

In certain embodiments, the anti-AREG antibodies can be prepared using well-established methods known in the art for developing monoclonal antibodies. In certain embodiment, the monoclonal antibodies are prepared using hybridoma technology. For hybridoma formations, a host animal (e.g., a mouse) is typically immunized with an immunizing agent (e.g., an AREG polypeptide) to elicit lymphocytes that produce or are capable of producing antibodies that will specifically bind to the immunizing agent. Alternatively, the lymphocytes can be immunized in vitro. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, rabbit, bovine and human origin. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. In certain embodiments, the immortalized cell lines are murine myeloma lines. In certain embodiments, myeloma cells can be subjected to genetic manipulation. For example, and without any limitation, genetic manipulation can be carried out using zinc-finger nuclease (ZFN) mutagenesis and Transcription Activator-Like Effector Nucleases (TALENs) mutagenesis.

In certain embodiments, the anti-AREG antibody is a monoclonal antibody isolated or purified from the culture medium or ascites fluid. In certain embodiments, the anti-AREG antibody is purified by immunoglobulin purification procedures including, without any limitation, protein A-Sepharose, hydroxylapatite chromatography, gel electrophoresis, dialysis, or affinity chromatography.

In certain embodiments, the anti-AREG antibody monoclonal antibodies can also be made by recombinant DNA methods. DNA encoding the anti-AREG monoclonal antibodies can be readily isolated and sequenced. The hybridoma cells of the present disclosure can serve as a preferred source of DNA. Once isolated, the DNA can be placed into expression vectors, which are then transfected into host cells. Host cells can include, but are not limited to, HEK293 cells, HEK293T cells, simian COS cells, Chinese hamster ovary (CHO) cells, and myeloma cells that do not otherwise produce immunoglobulin protein, to obtain the synthesis of monoclonal antibodies in the recombinant host cells. The DNA also can be modified, for example, and without any limitation, by substituting the coding sequence for human heavy and light chain constant domains in place of the homologous murine sequences (see U.S. Pat. No. 4,816,567) or by covalently joining to the immunoglobulin coding sequence all or part of the coding sequence for a non-immunoglobulin polypeptide. In certain embodiments, this non-immunoglobulin polypeptide can be substituted for the constant domains of an antibody of the present disclosure, or can be substituted for the variable domains of one antigen-combining site of an antibody of the present disclosure to create a chimeric bivalent antibody.

In certain embodiments, the anti-AREG antibody disclosed herein can be produced by various procedures known by those skilled in the art. For example, for the production of polyclonal antibodies in vivo, host animals (e.g., as rabbits, rats, mice, etc.) can be immunized with either free or carrier-coupled antigens (e.g., AREG polypeptide) by intraperitoneal and/or intradermal injection. In certain embodiments, injection material can be an emulsion containing about 100 μg of antigen or carrier protein. In certain embodiments, injection materials can include an adjuvant. Non-limiting examples of adjuvants encompassed by the present disclosure include Freund's (complete and incomplete), mineral gels such as aluminum hydroxide, surface-active substances such as lysolecithin, pluronic polyols, polyanions, peptides, oil emulsions, dinitrophenol, and potentially useful human adjuvants such as BCG and Corynebacterium parvum. In certain embodiments, the anti-AREG antibody can be selected and produced using high throughput methods of discovery. In certain embodiments, the anti-AREG antibody is produced through the use of display libraries. The term “display,” as used herein, refers to the expression or “display” of proteins or peptides on the surface of a given host. The term “library,” as used herein, refers to a collection of unique cDNA sequences and/or the proteins that are encoded by them. A library can contain from as little as two unique cDNAs to hundreds of billions of unique cDNAs. In certain embodiments, the anti-AREG antibody is a synthetic antibody produced using antibody display libraries or antibody fragment display libraries. The term “antibody fragment display library,” as used herein, refers to a display library wherein each member encodes an antibody fragment containing at least one variable region of an antibody. Display libraries can be expressed in several possible hosts including, but not limited to, yeast, bacteriophage, bacteria, and retroviruses.

In certain embodiments, Fab display libraries are expressed in yeasts or bacteriophages (also referred to herein as “phages” or “phage particles”). When expressed, the Fabs decorate the surface of the phage or yeast where they can interact with a given antigen (e.g., AREG). An AREG polypeptide or fragment thereof can be used to select phage particles or yeast cells expressing antibody fragments with the highest affinity for that antigen. The DNA sequence encoding the CDR of the bound antibody fragment can then be determined through sequencing using the bound particle or cell.

In yeast display, cDNA encoding different antibody fragments are introduced into yeast cells where they are expressed, and the antibody fragments are “displayed” on the cell surface as described by Chao et al. (Chao, G. et al., Isolating and engineering human antibodies using yeast surface display. Nat Protoc. 2006; 1(2):755-68). In yeast surface display, expressed antibody fragments can contain an additional domain that includes the yeast agglutinin protein, Aga2p. This domain allows the antibody fragment fusion protein to attach to the outer surface of the yeast cell through the formation of disulphide bonds with surface-expressed Agalp. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired antigenic target antigen, incubated with the cells. Target antigens can be covalently or otherwise modified with a chemical or magnetic group to allow for efficient cell sorting after successful binding with a suitable antibody fragment takes place. Recovery can be by way of magnetic-activated cell sorting (MACS), fluorescence-activated cell sorting (FACS), or other cell sorting methods known in the art. Once a subpopulation of yeast cells is selected, the corresponding plasmids can be analyzed to determine the CDR sequence.

As described above, after selection of a host expressing a high affinity antibody or antibody fragment, the coding regions from the antibody or antibody fragment can be isolated and used to generate whole antibodies, including human antibodies, or any other desired antigen binding fragment, and expressed in any desired host.

Non-limiting examples of techniques that can be used to produce antibodies and antibody fragments disclosed herein include those described in U.S. Pat. Nos. 4,946,778 and 5,258, 498; Miersch et al. (Miersch, S. et al., Synthetic antibodies: Concepts, potential and practical considerations. Methods. 2012 August; 57(4):486-98), Chao et al. (Chao, G. et al., Isolating and engineering human antibodies using yeast surface display. Nat Protoc. 2006; 1(2):755-68), Huston et al. (Huston, J. S. et al., Protein engineering of single-chain Fv analogs and fusion proteins. Methods Enzymol. 1991; 203:46-88); Shu et al. (Shu, L. et al., Secretion of a single-gene-encoded immunoglobulin from myeloma cells. Proc Natl Acad Sci USA. 1993 Sep. 1; 90(17):7995-9); and Skerra et al. (Skerra, A. et al., Assembly of a functional immunoglobulin Fv fragment in

Escherichia coli. Science. 1988 May 20; 240(4855): 1038-41), cach of which is incorporated herein by reference in its entirety.

Additional techniques for the production of antibodies can be found in Harlow and Lane “Antibodies, A Laboratory Manual”, Cold Spring Harbor Laboratory Press, 1988 and Harlow and Lanc “Using Antibodies: A Laboratory Manual” Cold Spring Harbor Laboratory Press, 1999.

In certain embodiments, the anti-AREG antibody can bind an AREG polypeptide with an affinity (K_{D}) from about 10⁻⁵ M or less to about 10⁻¹² M or less. In certain embodiments, the K_{D} can be from about 10⁻⁵ M or less to about 10⁻⁶ M or less, from about 10⁻⁵ M or less to about 10⁻⁷ M or less, from about 10⁻⁵ M or less to about 10⁻⁸ M or less, from about 10⁻⁵ M or less to about 10⁻⁹ M or less, from about 10⁻⁵ M or less to about 10⁻¹⁰ M or less, from about 10⁻⁵ M or less to about 10⁻¹¹ M or less, from about 10⁻⁶ Mor less to about 10⁻¹² M or less, from about 10⁻⁷ M or less to about 10⁻¹² M or less, from about 10⁻⁸ M or less to about 10⁻¹² M or less, from about 10⁻⁹ M or less to about 10⁻¹² M or less, from about 10⁻¹⁰ M or less to about 10⁻¹² M or less, from about 10⁻¹¹ M or less to about 10⁻¹²M or less. In certain embodiments, the K_{D} is about 10⁻⁵ M or less. In certain embodiments, the K_{D} is about 10⁻⁶ M or less. In certain embodiments, the K_{D} is about 10⁻⁷ M or less. In certain embodiments, the K_{D} is about 10⁻⁸ M or less. In certain embodiments, the K_{D} is about 10⁻⁹ M or less. In certain embodiments, the K_{D} is about 10⁻¹⁰ M or less. In certain embodiments, the K_{D} is about 10⁻¹¹ M or less. In certain embodiments, the K_{D} is about 10⁻¹² M or less.

### 3.1. Exemplified Anti-Amphiregulin Antibodies

In certain non-limiting embodiments, the anti-AREG antibody is an anti-AREG antibody disclosed in U.S. Pat. No. 10,640,556, the content of which is incorporated by reference in its entirety.

In certain embodiments, the anti-AREG antibody comprises a heavy chain comprising the amino acid sequence set forth in SEQ ID NO: 7. In certain embodiments, the anti-AREG antibody comprises a light chain comprising the amino acid sequence set forth in SEQ ID NO: 8. In certain embodiments, the heavy chain comprises a CDR1 comprising the amino acid sequence set forth in SEQ ID NO: 9. In certain embodiments, the heavy chain comprises a CDR2 comprising the amino acid sequence set forth in SEQ ID NO: 10. In certain embodiments, the heavy chain comprises a CDR3 comprising the amino acid sequence set forth in SEQ ID NO: 11. In certain embodiments, the light chain comprises a CDR1 comprising the amino acid sequence set forth in SEQ ID NO: 12. In certain embodiments, the light chain comprises a CDR2 comprising the amino acid sequence set forth in SEQ ID NO: 13. In certain embodiments, the light chain comprises a CDR3 comprising the amino acid sequence set forth in SEQ ID NO: 14. SEQ ID NOs: 7-14 are provided below:

(SEQ ID NO: 7)
EVQLQQSGAELVRSGASVKLSCTASGFNIKDSYMHWVKQRPEQGLEWIG
WVDPDNGDTEYAPEFQGRATLTADTFSSTAYLQLTSLTSEDTAVYYCNA
PSTYGHYGFAYWGQGTLVTVSA
(SEQ ID NO: 8)
DIVMTQAAPSVPVTPGESVSISCRSSKSLLHSNGKAYLYWFLQRPGQSP
QLLIYRMSNLASGVPDRFSGSGSGTAFTLRISRVEAEDVGVYYCMQHLE
YPLTFGAGTKLELKR
(SEQ ID NO: 9)
GFNIKDSY
(SEQ ID NO: 10)
VDPDNGDT
(SEQ ID NO: 11)
NAPSTYGHYGFAY
(SEQ ID NO: 12)
KSLLHSNGKAY
(SEQ ID NO: 13)
RMS
(SEQ ID NO: 14)
MQHLEYPLT

In certain embodiments, the anti-AREG antibody comprises a heavy chain comprising the amino acid sequence set forth in SEQ ID NO: 15. In certain embodiments, the anti-AREG antibody comprises a light chain comprising the amino acid sequence set forth in SEQ ID NO: 16. In certain embodiments, the heavy chain comprises a CDR1 comprising the amino acid sequence set forth in SEQ ID NO: 17. In certain embodiments, the heavy chain comprises a CDR2 comprising the amino acid sequence set forth in SEQ ID NO: 18. In certain embodiments, the heavy chain comprises a CDR3 comprising the amino acid sequence set forth in SEQ ID NO: 19. In certain embodiments, the light chain comprises a CDR1 comprising the amino acid sequence set forth in SEQ ID NO: 20. In certain embodiments, the light chain comprises a CDR2 comprising the amino acid sequence set forth in SEQ ID NO: 21. In certain embodiments, the light chain comprises a CDR3 comprising the amino acid sequence set forth in SEQ ID NO: 22. SEQ ID NOs: 15-22 are provided below.

(SEQ ID NO: 15)
EVQLQQSGAELVKPGASVKLSCTASGFNIKDTYMHWVKQRPEQGLEWIG
RIDPANRSTKYDPKFQGKATITADTSSNTADLHLSSLTSEDTAVYYCAR
LYGDSVWYFDVWGAGTTVTVSSAKT
(SEQ ID NO: 16)
QIVLTQSPAILSASPGEKVTMTCRAGSSVNYIHWYQQKPGSSPKPWIYA
TSNLASGVPARFSGSGSGTSYSLTISRVEAEDAATYYCQQWSGYPPMLT
FGAGTKLELKR
(SEQ ID NO: 17)
GFNIKDTY
(SEQ ID NO: 18)
IDPANRST
(SEQ ID NO: 19)
ARLYGDSVWYFDV
(SEQ ID NO: 20)
SSVNY
(SEQ ID NO: 21)
ATS
(SEQ ID NO: 22)
QQWSGYPPMLT

In certain embodiments, the anti-AREG antibody comprises a heavy chain comprising the amino acid sequence set forth in SEQ ID NO: 23. In certain embodiments, the anti-AREG antibody comprises a light chain comprising the amino acid sequence set forth in SEQ ID NO: 24. In certain embodiments, the heavy chain comprises a CDR1 comprising the amino acid sequence set forth in SEQ ID NO: 25. In certain embodiments, the heavy chain comprises a CDR2 comprising the amino acid sequence set forth in SEQ ID NO: 26. In certain embodiments, the heavy chain comprises a CDR3 comprising the amino acid sequence set forth in SEQ ID NO: 27. In certain embodiments, the light chain comprises a CDR1 comprising the amino acid sequence set forth in SEQ ID NO: 28. In certain embodiments, the light chain comprises a CDR2 comprising the amino acid sequence set forth in SEQ ID NO: 29. In certain embodiments, the light chain comprises a CDR3 comprising the amino acid sequence set forth in SEQ ID NO: 30. SEQ ID NOs: 23-30 are provided below.

(SEQ ID NO: 23)
EVQLVESGGDLVKPGGSLKLSCAASGFTFSNSGMXWERLTPDKRLEWVA
TISSGSTYTFYPDTVKGRFIISRXNAKNTLYLQMSSLKSEDTAIYYCVR
EIWPVWGAGTTITVSSAKT
(SEQ ID NO: 24)
QAVVTQESALSTSPGETVTLTCRSSTGAVTTSNYANWVQEKPDHLFTGL
LGDTDNRPPGVPARFSGSLLGDKAALTITGAQTEDEAIYFCALWYSNHW
VFGGGTKLTVL
(SEQ ID NO: 25)
GFTFSNSG
(SEQ ID NO: 26)
ISSGSTYT
(SEQ ID NO: 27)
VREIWPV
(SEQ ID NO: 28)
TGAVTTSNY
(SEQ ID NO: 29)
DTD
(SEQ ID NO: 30)
ALWYSNHWV

### 4. Pharmaceutical Compositions and Methods of Treatment

The present disclosure provides methods of treating a subject having cancer, including administering an IL33 polypeptide and an anti-AREG antibody in the subject.

In certain embodiments, the methods disclosed herein can be used for treating any suitable cancers. Non-limiting examples of cancers encompassed by the disclosed subject matter include liver cancers, brain cancers, cervical cancers, colorectal cancers, breast cancers, endometrial carcinomas, gastric cancers, cancers of the head and neck, bladder cancers, lung cancers, ovarian cancers, biliary trec cancers, hepatocellular carcinomas, leukemia, lymphoma, mycloma, and sarcoma. In certain embodiments, methods disclosed herein can be used for treating a cancer selected from bladder urothelial carcinoma, cervical squamous cell carcinoma and endocervical adenocarcinoma, cholangiocarcinoma, colon adenocarcinoma, head and neck squamous cell carcinoma, kidney chromophobe, kidney renal papillary cell carcinoma, liver hepatocellular carcinoma, lung adenocarcinoma, lung squamous cell carcinoma, prostate adenocarcinoma, rectum adenocarcinoma, stomach adenocarcinoma, and uterine corpus endometrial carcinoma. In certain embodiments, methods disclosed herein can be used for treating a colon cancer, gastric cancer, breast cancer, lung cancer, pancreatic cancer, head and neck cancer, ovarian cancer, melanoma, and combinations thereof.

In certain embodiments, the subject is a human subject. In certain embodiments, the subject is a non-human subject, such as, but not limited to, a non-primate, a dog, a cat, a horse, a rabbit, a mouse, a rat, a guinea pig, a fowl, a cow, a goat, or a sheep.

In certain embodiments, the IL33 polypeptide and the anti-AREG antibody disclosed herein can be administered to the subject by any suitable route known in the art, including, but not limited to, oral, parenteral, topical, intravenous, subcutaneous, intraperitoneal, intrapulmonary, intranasal, and/or intralesional, intra-arterial, or intrathecal. Parenteral infusions include intramuscular, intravenous, intraarterial, intraperitoneal, or subcutaneous administration.

In certain embodiments, the IL33 polypeptide and the anti-AREG antibody can be physically combined prior to administration, administered by the same route, or be administered over the same time frame. In certain embodiments, the first, second, and/or third microparticles are not physically combined prior to administration, administered by the same route, or are not administered over the same time frame. In certain embodiments, the IL33 polypeptide and the anti-AREG antibody disclosed herein are included in pharmaceutical compositions to be administered to the subject. In certain embodiments, the pharmaceutical compositions further include a pharmaceutically acceptable carrier. Suitable pharmaceutically acceptable carriers that can be used with the presently disclosed subject matter have the characteristics of not interfering with the effectiveness of the biological activity of the active ingredients, e.g., anti-AREG antibodies, and that is not toxic to the subject to whom it is administered. Non-limiting examples of suitable pharmaceutical carriers include phosphate-buffered saline solutions, water, emulsions, such as oil/water emulsions, various types of wetting agents, and sterile solutions. Additional non-limiting examples of pharmaceutically acceptable carriers include gels, bioabsorbable matrix materials, implantation elements containing the inhibitor, and/or any other suitable vehicle, delivery, or dispensing mechanism or material. Such pharmaceutically acceptable carriers can be formulated by conventional methods and can be administered to the subject. In certain embodiments, the pharmaceutical acceptable carriers can include buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, the suitable pharmaceutically acceptable carriers can include one or more of water, saline, phosphate buffered saline, dextrose, glycerol, ethanol, or combinations thereof.

In certain non-limiting embodiments, the pharmaceutical compositions of the present disclosure can be formulated using pharmaceutically acceptable carriers well known in the art that are suitable for oral administration. Such carriers can enable the pharmaceutical compositions to be formulated as tablets, pills, capsules, liquids, gels, syrups, slurries, suspensions and the like, for oral or nasal ingestion by a subject to be treated. In certain embodiments, the pharmaceutical composition is formulated as a capsule. In certain embodiments, the pharmaceutical composition can be a solid dosage form. In certain embodiments, the tablet can be an immediate release tablet. Alternatively or additionally, the tablet can be an extended or controlled release tablet. In certain embodiments, the solid dosage can include both an immediate release portion and an extended or controlled release portion.

In certain embodiments, the pharmaceutical compositions suitable for use in the presently disclosed subject matter can include compositions where the active ingredients, e.g., anti-AREG antibodies, are contained in an effective amount. The effective amount of an active ingredient can vary depending on the active ingredient, compositions used, the cancer and its severity, and the age, weight, etc., of the subject to be treated. In certain embodiments, a subject can receive an effective amount of the active ingredient in single or multiple administrations of one or more composition, which can depend on the dosage and frequency as required and tolerated by the subject.

In certain embodiments, the pharmaceutical compositions further include a second anti-cancer agent as disclosed below.

In certain embodiments, methods disclosed herein employ a genetic engineering system to increase (e.g., delivering) the expression of IL33 polypeptides, or a functional fragment thereof. In certain embodiments, methods disclosed herein employ a genetic engineering system to increase (e.g., delivering) the expression of anti-AREG antibodies, or a functional fragment thereof. Non-limiting examples of the genetic engineering system include viral vectors and non-viral vectors comprising a nucleic acid sequence encoding for IL33 polypeptide, or a functional fragment thereof.

The genetic engineering system disclosed herein can be delivered into a mammalian cell using a viral vector, e.g., retroviral vectors such as gamma-retroviral vectors, and lentiviral vectors. Combinations of viral vector and an appropriate packaging line can be suitable, where the capsid proteins will be functional for infecting human cells. Various amphitropic virus-producing cell lines are known, including, but not limited to, PA12 (Miller, et al. (1985) Mol. Cell. Biol. 5:431 -437); PA317 (Miller, et al. (1986) Mol. Cell. Biol. 6:2895-2902); and CRIP (Danos, et al. (1988) Proc. Natl. Acad. Sci. USA 85:6460-6464). Non-amphitropic particles can be suitable too, e.g., particles pseudotyped with VSVG, RD114, or GALV envelope and any other known in the art. Methods of transduction can also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the mammalian cell disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In certain embodiments, the viral vectors are oncolytic viral vectors that target cancer cells and deliver the genetic engineering system to the cancer cells. Non-limiting examples of oncolytic viral vectors are disclosed in Lundstrom et al., Biologics. 2018; 12: 43-60, and the content of which is incorporated by reference herein in its entirety. In certain embodiments, the oncolytic viral vectors are selected from adenoviruses, HSV, alphaviruses, rhabdoviruses, Newcastle disease virus (NDV), vaccinia viruses (VVs), and combinations thereof.

Non-viral approaches can also be employed for genetic engineering of the mammalian cell disclosed herein. For example, a nucleic acid molecule can be introduced into the mammalian cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be beneficial for the delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cells (or its descendants) are injected into a targeted tissue or are injected systemically.

In certain embodiments, non-viral approaches include nanotechnology-based approaches, which use non-viral vectors. The non-viral vectors can be made of a variety of materials, including inorganic nanoparticles, carbon nanotubes, liposomes, protein and peptide-based nanoparticles, as well as nanoscale polymeric materials. Riley et al., Nanomaterials (Basel). 2017 May; 7(5): 94 reviews nanotechnology-based methods for delivery of a nucleic acid molecule to a subject, the content of which is incorporated by reference in its entirety.

In certain embodiments, non-viral approaches include nanoparticles. As used herein, a “nanoparticle” refers to any particle having a diameter of less than 1000 nm, e.g., about 10 nm to about 200 nm. In certain embodiments, the nanoparticles can have a diameter of about 10 nm to about 90 nm, or about 20 nm to about 80 nm, or about 60 nm to about 120 nm, or about 70 nm to about 120 nm, or about 80 nm to about 120 nm, or about 90 nm to about 120 nm, or about 100 nm to about 120 nm, or about 60 nm to about 130 nm, or about 70 nm to about 130 nm, or about 80 nm to about 130 nm, or about 90 nm to about 130 nm, or about 100 nm to about 130 nm, or about 110 nm to about 130 nm, or about 60 nm to about 140 nm, or about 70 nm to about 140 nm, or about 80 nm to about 140 nm, or about 90 nm to about 140 nm, or about 100 nm to about 140 nm, or about 110 nm to about 140 nm, or about 60 nm to about 150 nm, or about 70 nm to about 150 nm, or about 80 nm to about 150 nm, or about 90 nm to about 150 nm, or about 100 nm to about 150 nm, or about 110 nm to about 150 nm, or about 120 nm to about 150 nm.

In certain embodiments, the nanoparticles can comprise a core. In certain embodiments, the core comprises a nucleic acid encoding for the IL33 polypeptide or a functional fragment thereof. In certain embodiments, the core comprises a nucleic acid encoding for the anti-AREG antibody or a functional fragment thereof.

In certain embodiments, the nanoparticle can comprise one or more lipids. In certain embodiments, the lipids can be neutral, anionic, or cationic at physiological pH. In certain embodiments, the lipids can be sterols. For example, in certain embodiments, the lipid nanoparticle can comprise cholesterol, phospholipids, and sphingolipids. In certain embodiments, the nanoparticles comprise PEGylated derivatives of neutral, anionic, and cationic lipids. The incorporation of PEGylated derivatives can improve the stability of the nanoparticles. Non-limiting examples of PEGylated lipids include distearoylphosphatidylethanlamine-polyethylene glycol (DSPE-PEG), stearyl-polyethylene glycol, and cholesteryl-polyethylene glycol. In certain embodiments, the nanoparticle can comprise substituted or unsubstituted fatty acids. Non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, hencicosanoic acid, bchenic acid, tricosanoic acid, lignoceric acid, pentacosanoic acid, cerotic acid, heptacosanoic acid, montanic acid, nonacosanoic acid, melissic acid, henatriacontanoic acid, lacceroic acid, psyllic acid, geddic acid, ceroplastic acid, hexatriacontanoic acid, and combinations thereof. Non-limiting examples of unsaturated fatty acids include hexadecatrienoic acid, alpha-linolenic acid, stearidonic acid, cicosatrienoic acid, cicosatetraenoic acid, cicosapentaenoic acid, heneicosapentaenoic acid, docosapentaenoic acid, docosahexaenoic acid, tetracosapentaenoic acid, tetracosahexaenoic acid, linoleic acid, gamma-linolenic acid, cicosadienoic acid, dihomo-gamma-linolenic acid, arachidonic acid, docosadienoic acid, adrenic acid, docosapentaenoic acid, tetracosatetraenoic acid, tetracosapentaenoic acid, oleic acid, cicosenoic acid, mead acid, crucic acid, nervonic acid, rumenic acid, α-calendic acid, β-calendic acid, jacaric acid, α-eleostearic acid, β-cleostearic acid, catalpic acid, punicic acid, rumelenic acid, aαparinaric acid, β-parinaric acid, bosscopentacnoic acid, pinolenic acid, podocarpic acid, palmitoleic acid, vaccenic acid, gadoleic acid, crucic acid, and combinations thereof.

In certain embodiments, the nanoparticles can comprise polymers. In certain embodiments, the polymer can be amphiphilic, hydrophilic, or hydrophobic. In certain embodiments, the polymer can be biocompatible, e.g., the polymer does not induce an adverse and/or inflammatory response when administered to a subject. For example, without limitation, a polymer can be selected from polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), poly(lactic) acid-co-poly(glycolic) acid (PLGA), polycaprolactone, or copolymers or derivatives including these and/or other polymers. In certain embodiments, the polymer can contain PEG.

In certain embodiments, the nanoparticles can comprise cationic polymers. In certain embodiments, the cationic polymers can be branched or linear. Cationic polymers can condense and protect negatively charged molecules such as DNA or RNA. In certain embodiments, without limitation, the cationic polymers can be polyethylenimines, poly-histidyl polymers, chitosan, poly(amino ester glycol urethane), polylysines, amino cyclodextrin derivatives. In certain embodiments, the nanoparticle comprises linear polyethylenimine. Additional information on nanoparticles comprising linear polyethylenimine can be found in International Patent Application Nos. PCT/IB2008/002339 and PCT/IB2008/055256, the content of which is incorporated in their entirety.

In certain embodiments, the nanoparticle can show organ tropism and can have an organ-specific distribution. For example, without limitation, the nanoparticle can deliver the nucleic acid to hepatic cells, lung cells, tumor cells, or immune cells. In certain embodiments, the nanoparticle can be conjugated to a ligand. In certain embodiments, the ligand can be mannose. In certain embodiments, the nanoparticle can show cell tropism by binding the ligand to a specific molecule on the cell. In certain embodiments, the cell can be a cancer cell. In certain embodiments, the cell can be myeloid. For example, without any limitation, the nanoparticle can be conjugated to mannose and can bind to a cell expressing a mannose-receptor (e.g., macrophages, dendritic cells) In certain embodiments, the nanoparticles can be biodegradable or non-biodegradable. In certain embodiment, the nanoparticle can be comprised in a pharmaceutical composition.

In certain embodiments, the presently disclosed subject matter comprises compositions comprising a nucleic acid sequence encoding the IL33 polypeptide disclosed herein. In certain embodiments, the nucleic acid sequence encoding the IL33 polypeptide is operably linked to a promoter. Non-limiting examples of promoters encompassed by the present disclosure include clongation factor (EF)-1 promoter, CMV promoter, SV40 promoter, PGK promoter, long terminal repeat (LTR) promoter, and metallothioncin promoter.

In certain embodiments, the genetic engineering system is administered to the subject in vivo, and thus increases the expression of the IL33 polypeptide.

In certain embodiments, methods disclosed herein further include administering a second anti-cancer treatment to the subject. Non-limiting exemplary anti-cancer treatments include chemotherapy, radiation therapy, targeted drug therapy, immunotherapy, immunomodulatory agents, cytokines, monoclonal and polyclonal antibodies, and any combinations thereof.

Non-limiting examples of second anti-cancer treatments include chemotherapeutic treatments, radiotherapeutic treatments, anti-angiogenic treatments, apoptosis-inducing treatments, anti-cancer antibodies, anti-cyclin-dependent kinase agents, and/or treatments which promote the activity of the immune system including but not limited to cytokines such as but not limited to interleukin 2, interferon, anti-CTLA4 antibody, anti-PD-1 antibody, and/or anti-PD-L1 antibody. In certain embodiments, the IL33 polypeptide, the anti-AREG antibody, and the one or more anti-cancer treatments are administered to a subject as part of a treatment regimen or plan.

In certain embodiments, the IL33 polypeptide, the anti-AREG antibody, and the one or more anti-cancer treatments can be physically combined prior to administration, administered by the same route, or be administered over the same time frame. In certain embodiments, the IL33 polypeptide, the anti-AREG antibody, and the one or more anti-cancer treatments are not physically combined prior to administration, administered by the same route, or are not administered over the same time frame. In certain embodiments, the IL33 polypeptide, the anti-AREG antibody, and the one or more anti-cancer treatments are not administered over the same time frame.

In certain embodiments, the second anti-cancer treatment is chemotherapy, which includes administering a chemotherapeutic agent to the subject. Any suitable chemotherapeutic agents known in the art can be used with the presently disclosed methods. Non-limiting examples of chemotherapeutic agents that can be used with the presently disclosed methods include acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azctepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; cdatrexate; cflornithine hydrochloride; elsamitrucin; enloplatin; enpromate; cpipropidine; epirubicin hydrochloride; erbulozole; esorubicin hydrochloride; estramustine; estramustine phosphate sodium; ctanidazole; ctoposide; ctoposide phosphate; ctoprine; fadrozole hydrochloride; fazarabine; fenretinide; floxuridine; fludarabine phosphate; fluorouracil; fosquidonc; fostriccin sodium; gemcitabine; gemcitabinc hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantronc hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantronc hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; analogues and derivative thereof; and combinations thereof.

In certain embodiments, the chemotherapeutic agent used with the presently disclosed methods includes one or more agent selected from cisplatin, carboplatin, docetaxel, gemcitabine, paclitaxel, paclitaxel, vinorelbine, pemetrexed, analogs and derivatives thereof, and combinations thereof.

In certain embodiments, the second anti-cancer treatment is an immunotherapy (also known as immuno-oncology) that uses components of the immune system. Non-limiting examples of immunotherapies include immune checkpoint inhibitors, adoptive T cell transfer, therapeutic antibodies, cancer vaccines, cytokines, Bacillus Calmette-Guérin (BCG), and any combinations thereof.

In certain embodiments, the second anti-cancer treatment includes administering an immune checkpoint inhibitor to the subject. In certain embodiments, the immune checkpoint inhibitor is selected from anti-PD1 antibodies, anti-PD-L1 antibodies, anti-CTLA-4 antibodies, and any combinations thereof. Non-limiting examples of anti-PD1 antibodies include pembrolizumab (Keytruda®), nivolumab (Opdivo®), cemiplimab (Libtayo®), and combinations thereof. Non-limiting examples of anti-PD-L1 antibodies include atezolizumab (Tecentriq®), avelumab (Bavencio®), durvalumab (Imfinzi®), and combinations thereof. Non-limiting examples of anti-CTLA-4 antibodies include ipilimumab (Yervoy®).

In certain embodiments, the immune checkpoint inhibitor is directed against one or more immune checkpoint modulators. For example, without limitation, immune checkpoint inhibitors can target AMHRII, B7-H3, B7-H4, BTLA, BTNL2, Butyrophilin family, CD27, CD28, CD30, CD40, CD40L, CD47, CD48, CD70, CD80, CD86, CD155, CD160, CD226, CD244, CEACAM6, CLDN6, CCR2, CTLA4, CXCR4, GD2, GGG (guanylyl cyclase G), GIRT, GIRT ligand, HHLA2, HVEM, ICOS, ICOS ligand, IFN, ILI, ILI R, IL1 RAP, IL6, IL6R, IL7, IL7R, IL12, IL12R, IL15, IL15R, LAG 3, LIGHT, LIF, MUC16, NKG2A family, 0X40, 0X40 ligand, PD1, PDL1, PDL2, Resokine, SEMA4D, Siglec family, SIRPalpha, STING, TGFbeta family, TIGIT, TIM3, TMIGD2, TNFRSFm VISTA, 4-1BB, and 4-1BB ligand.

### 5. Kits

The present disclosure provides kits for treating a subject having a cancer. In certain embodiments, the kits include an effective amount of an IL33 polypeptide. In certain embodiments, the kits include an effective amount of an anti-AREG antibody. In certain embodiments, the kits include a sterile container that contains the agents or the genetic engineering system; such containers can be boxes, ampules, bottles, vials, tubes, bags, pouches, blister-packs, or other suitable container forms known in the art. In certain embodiments, the kit can include a single dose of the agents or a pharmaceutical formulation thereof or multiple doses of the agents or a pharmaceutical formulation thereof. Such containers can be made of plastic, glass, laminated paper, metal foil, or other materials suitable for holding medicaments. A container can be any receptacle and closure suitable for storing, shipping, dispensing, and/or handling a pharmaceutical product.

In certain embodiments, the kits include instructions for administering the IL33 polypeptide and the anti-AREG antibody to a subject having a cancer. The instructions can include information about the use of the agent, composition, or genetic engineering system for treating the cancer. In certain embodiments, the instructions include at least one of the following: description of the components (e.g., the IL33 polypeptide and the anti-AREG antibody disclosed herein); dosage schedule and administration for treating the cancer; precautions; warnings; indications; counter-indications; over dosage information; adverse reactions; animal pharmacology; clinical studies;

and/or references. The instructions can be printed directly on the container (when present), or as a label applied to the container, or as a separate sheet, pamphlet, card, or folder supplied in or with the container.

In certain embodiments, the kit further includes instructions or supporting material that describe the use of the kit to diagnose a cancer and/or reference to a website or publication describing the same. In certain embodiments, the kit further includes instructions or supporting material that describe the use of the kit to determine a prognosis of cancer and/or reference to a website or publication describing the same. In certain embodiments, the kit further includes instructions or supporting material that describe the use of the kit to predict or monitor a subject's responsiveness to an anti-cancer treatment and/or reference to a website or publication describing the same. In certain embodiments, the instructions further include selecting an effective anti-cancer treatment based on the prediction or monitoring results.

In certain embodiments, the kit can further include a device for administering the agents or a pharmaceutical formulation thereof. For example, but not by way of limitation, the device can include a syringe, catheter, e.g., implantable catheter, and/or pump.

### EXAMPLE 1

The present disclosure will be better understood by reference to the following Example, which is provided as exemplary of the presently disclosed subject matter, and not by way of limitation.

### Materials and Methods

The present example provides experimental procedures that were followed to complete studies disclosed herein.

Mouse. C57BL/6J (000664), Foxp3YFP-cre (016959) mice were purchased from The Jackson Laboratory. The ST2 flox (Strain ID: Il1rl1tm1a(KOMP)) mice were purchased from KOMP Repository (UC Davis). All mice are on the background of C57BL/6. Mice were housed properly in the specific-pathogen-free (SPF) facility in the School of Medicine, University of Pittsburgh. All mice experiments were performed under the approval of the Institutional Animal Care and Use Committee (IACUC) of the University of Pittsburgh.

Cell lines and animal models. MC-38 cell line was cultured in DMEM with 10% fetal bovine serum (FBS) and 1% penicillin-streptomycin (P.S). Generation of B16-IL-33 and B16-vec tumor cell lines were previously reported. B16 and B16-IL-33 cells were cultured in RPMI medium with 10% FBS and 1% P.S. 1 million MC-38 cells were injected intradermally (i.d.) into the right flank of the mice. For B16 models, 0.1 million cells were injected i.d. IL-33 protein (10μg) was injected intraperitoneally (i.p.) on the 5th day after tumor inoculation, for a total of four times with 4-day intervals.

Single-cell isolation. Tumor tissues were processed according to the protocol described before previously. Mice were sacrificed, and tissues were freshly dissected. Tumor tissues were then cut into pieces and digested in serum serum-free RPMI with 0.33 mg/ml DNase and 0.25 mg/ml Liberase TL (Roche) and then ground, washed in PBS, and filtered through a 70 μm cell strainer for single-cell suspensions.

Flow cytometry. Flow cytometry experiments were all done by the instrument Aurora (Cytek Biosciences) from the flow core in the University of Pittsburgh and analyzed by Flowjo (BD). CD45 (clone 30-F11), CD4 (clone RMT4-5), CD8a (clone 53.67) were purchased from BD Bioscience or Biolegend. For intracellular transcription factors and cytokines staining, cells were stimulated with a leukocyte activation cocktail (BD) for 6 hours and then followed the standard staining protocol described before previously.

Droplet-based single-cell RNA-seq. CD4+ and CD8+ T cells were sorted and washed twice in PBS with 400μg/ml bovine serum albumin (BSA) and then resuspended with the concentration of 1,000-2,000 cells/μl. 18,000 cells were loaded into the Chromium single-cell A chip to generate

Gel Bead-In Emulsions using the 10× Genomics 5′ RNA single-cell method. Chromium Single Cell 5′ and Chromium Single Cell V(D)J Reagent Kits (10× Genomic, No. CG000086) were used to generate single-cell sequencing libraries. Libraries were sequenced on an Illumina NovaSeq6000 SP platform with 40,000 reads per cell on average.

CITE-seq. CITE-seq and Cell Hashing antibodies were used to achieve a single-cell level protein expression data and demultiplexing of different samples. In brief, single cell suspensions from tumor tissues were washed with staining buffer (PBS, 2% BSA, 0.01% Tween) and incubated with Fc blocker (Biolegend, Trustain FcX) for 10 mins on ice. Antibody cocktails were prepared by mixing CITE-seq antibodies, Cell hashing tags, and fluorescent fluorescent-labeled antibodies with pre-optimized concentrations. Antibody cocktails were applied, and cells were incubated on ice for 30 mins. Stained cells were washed three times using staining buffer, then CD4+ and CD8+ T cells from different treatment groups were sorted out and pooled immediately before loading to a 10× single cell platform. A 10× Chromium Single Cell 5′ V(D)J Reagent Kit (10x Genomics,

No. CG000186) was used to construct the cell surface protein libraries. Libraries were sequenced on an Illumina NovaScq6000 SP platform with 10,000 reads per cell on average.

SCENIC. The SCENIC pipeline (R package, v.1.1.2.2) was used to construct and score gene regulatory networks (regulons). Each regulon was composed of a transcription factor and its putative target genes. The output of SCENIC was a matrix of the activity of regulons, where rows corresponded to regulons and columns corresponded to cells. Single-cell RNA-seq data processing. The Seurat (3.1.5) R package was utilized to identify clusters and find DEGs. The Seurat object was set up by filtering out genes expressed on less than 4 cells and cells with less than 200 detected features. Cells with unique feature counts between 200 and 2,500 and less than 5% mitochondrial counts were selected for further analysis. Following the standard QC workflow, the unique molecular identified (UMI) count matrix was log-transformed, normalized, and scaled using default parameters. Single-cell RNA-seq dataset dimension reduction. Seurat version 3.1.5 functions: Find Variable Features, RunPCA, and RunUMAP were used to calculate top variable genes, PCA, and UMAP, respectively. The top 2,000 features were used to calculate PCA and utilized the top 50 principal components (PCs) to generate UMAP visualizations. Shared nearest neighbor (SNN) clustering method was performed by Seurat's Find Clusters function based on the top 50 PCs, with resolution set to 0.8˜1.4.

Identify gene expression modules (GEMs) using the nHDP model. The nested hierarchical Dirichlet processes (nHDP) model on mouse single-cell RNA-seq data was used to identify the gene expression modules which potentially indicate different biological processes. The nHDP model was originally designed to model co-occurrence patterns of words in text documents in the text mining domain, which is a close analog to co-expression expression of genes (words) in single cells (documents). Furthermore, it hierarchically organized GEMs in a tree, so that the GEMs close to the root were expressed in a broad range of cells, whereas the GEMs at the leaves of the tree were only expressed in highly specialized (differentiated) cells. A three-layer hierarchical tree structure was designed, with branching factors of 5, 4, and 3 from the root to the 2nd and 3rd layers leading to 85 nodes/gene expression modules (GEMs) in total. Each GEM defined a distribution over the space of genes reflecting the information on which genes were commonly assigned to the module. The nHDP model generated 1) a ranked list of genes most commonly assigned to GEMs and 2) a cell-by-GEM count matrix, of which an element reflected the number of genes expressed in a given cell and that was assigned to a specific GEM.

Test differentially expression of a GEM between subpopulations of single cells. The non-parametric Wilcoxon rank-sum test was used to test whether a GEM was differentially expressed between know-out cells and wild-type cells. A detection threshold (alpha) was set at 0.05 when assessing whether the GEM was significantly differentially expressed.

Estimate the enrichment of a GEM in TCGA tumors. The gene set variation analysis (GSVA) method was applied to estimate the enrichment of a GEM in a tumor relative to a cohort of tumor samples. The input of the GSVA was the bulk RNA expression of genes in tumors and a gene list containing the top 50 genes associated with a GEM. The output of the GSVA was the relative enrichment scores of a GEM in tumors. The distribution of the GSVA scores among the input tumor samples generally followed a normal distribution in the range of −3 to 3. The score reflected, relatively to other tumors, the enrichment of cells expressing a GEM in a tumor.

Testing causal hypotheses through conditional independence. Two hypotheses of the causal relationships among IL33, ST2 and GEM16: 1) expression of IL33; formation of IL33-ST2 signal; expression of GEM16 (FIGS. 16), and 2) expression of ST2; formation of IL33-ST2 signal, expression of GEM16 (FIG. 16). Under this scenario, there is no latent variable regulating both IL33 and GEM16. To assess the causal relationships, IL33 and GEM 16 were tested for statistical independent conditioning on IL33-ST2 protein complex and whether ST2 and GEM16 were statistically independent conditioning on IL33-ST2 protein complex. Expression values mRNA IL33 and ST2 products were used to estimate the concentration of the IL33-ST2 protein complex. To test the conditional independence, a kernel conditional independence test was used, available as the R package called CondIndTest. The inputs of the test were approximated using mRNA measurements as surrogates-protein concentrations of ST2, IL33, IL33-ST2 complex, and the enrichment score of GEM16 for a certain tumor type. The hypothesis to be tested was that variable X and variable Y were conditionally independent given the variable Z. If the p-value of the test was less than 0.05, the hypothesis of independence was rejected, i.e., the X and Y were dependent conditioning on Z.

Re-analysis of ATAC-seq data. AREG expression in ST2 Treg cells and other Treg cells was analyzed in publicly available ATAC-seq datasets (GSE130884) of sorted tissue ST2Treg cells and other Treg cells from lung tissue. ATAC-seq tracks were visualized in the Integrative Genomics Viewer (IGV, V2.7.2).

Statistical analysis. Statistical analysis was performed using Graphpad Prism v8 software. Values were reported as mean ±SEM. P-value was calculated by two-tailed student's t-test when comparing two groups, and the log-rank (Mantel-Cox) test for comparing several Kaplan-Meier survival curves. Two-way ANOVA was used for comparing tumor growth curves. Exact one-proportion z test was used for comparing the proportions of different samples. * p<0.05; ** p<0.01; *** p<0.001; **** p<0.0001.

### IL-33 Expression in the Tumors Drove Robust CD8+ T Cell Immune Responses

The present example dissected the immune regulatory cellular network in IL-33-expressing tumors. scRNAseq analysis was employed to reveal the cellular composition and genetic programming of tumor-infiltrating T lymphocytes in tumors that expressed a high level of IL-33. Molecular characteristics and the function of IL-33 signaling on Treg cells and its impact on antitumor efficacy were studied. Further, it was investigated the underlying mechanism of how IL1RL1⁺ Treg and its cellular partners are involved in establishing immune suppression in the TME.

Here, it is demonstrated that the injection of IL-33 protein and the expression of secreted IL-33 inhibited tumor growth. Antitumor efficacy of IL-33 was dependent on intact host IL1rl1 (see FIGS. 8A and 8B). To understand how IL-33 drives cellular immunity in the tumor microenvironment (TME), single-cell RNA sequencing (scRNA-seq) and paired TCR repertoire profiling of tumor-infiltrating TCR-β⁺ T cells, from B16 and IL-33-expressing B16 (B16-IL-33) tumors (see FIG. 8C), were performed. A dimension reduction using uniform manifold approximation and projection (UMAP) followed by unsupervised clustering identified 12 clusters of T cells. The clusters were composed of three main lineage populations: a) conventional CD4³⁰ T cells, b) CD4⁺Foxp3⁺ regulatory T cells (Treg), and c) CD8⁺ T cells (see FIGS. 8D-8F). Notably, the fraction of CD8⁺ T cells and Treg cells was significantly increased in the B16-IL33 tumors (see FIG. 8G).

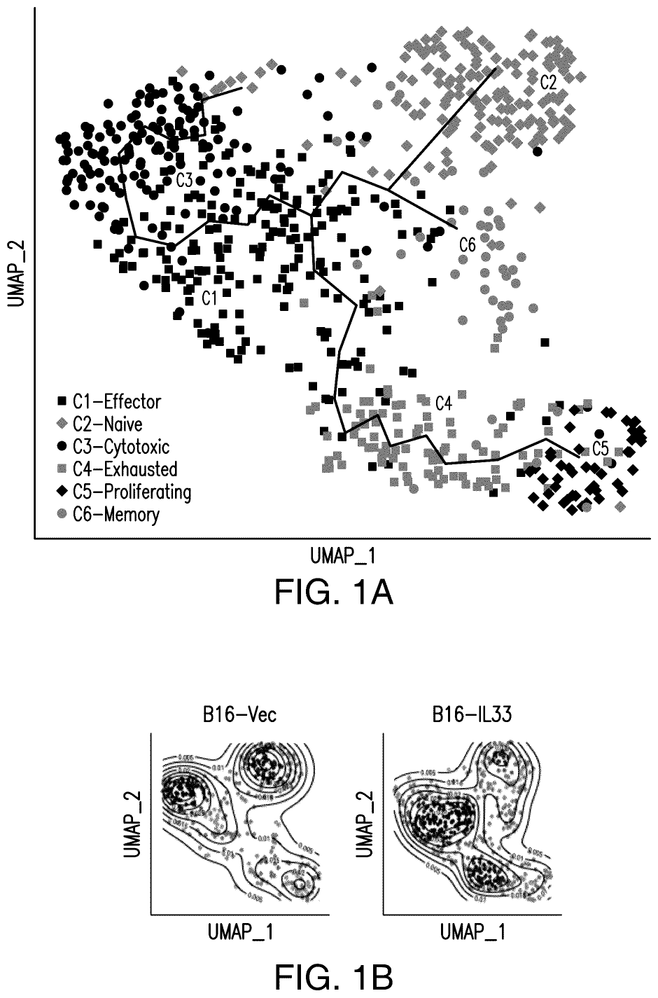

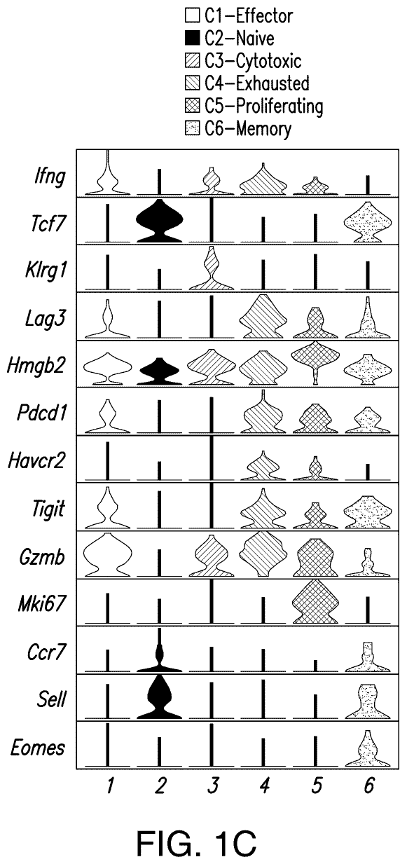

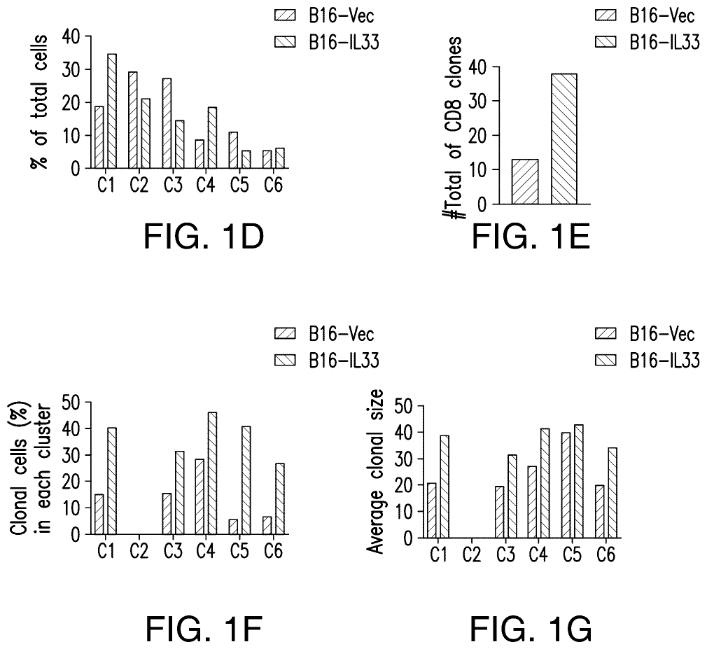

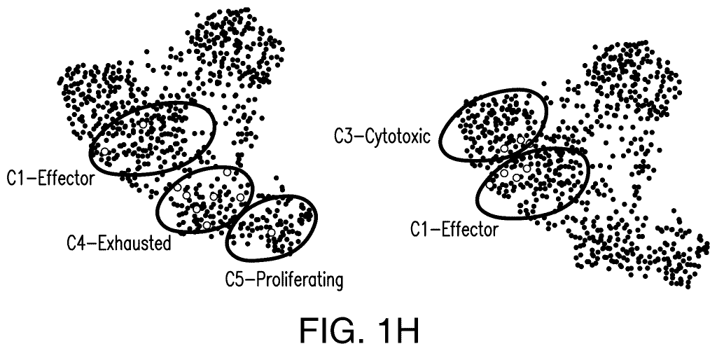

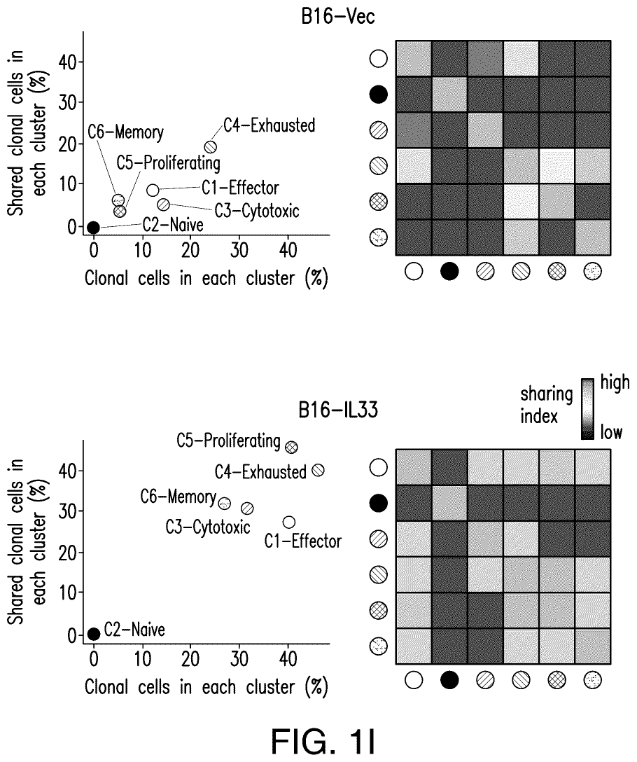

The present example also explored CD8⁺ T cells in the TME. Notably, a sub-clustering of CD8⁺ T cells revealed six populations, namely effector, naïve, cytotoxic, exhausted, proliferating, and memory (clusters 1 to 6, see FIGS. 1A-1C and FIGS. 9A-9F). Hierarchical clustering revealed that naïve and memory CD8⁺ T cells expressed similar naïve/stem-like markers such as Tcf-7, Sell, and Ccr7 (see FIGS. 1C and 9B). Effector and cytotoxic CD8⁺ T cells expressed similar effector genes such as Ifng and GzmB, and had downregulated expression of immune regulatory molecules such as Pdcd1, Haver2, and Lag3 (see FIGS. 1C and 9A). Exhausted and proliferating CD8⁺ T cells expressed high levels of immune regulatory molecules such as Pdcd1, Havcr2, Tigit, and Lag3 (see FIGS. 1C and 9B). A trajectory analysis showed three distinct pathways of CD8⁺ T cell differentiation in these experimental settings (i.e., naïve→memory, naïve→effector→exhausted→proliferative, and naïve→effector→cytotoxic) (see FIGS. 1A, 9E and 9F). The analysis further showed that IL-33 led to a drastic change in the composition of the CD8⁺ T cell subsets. The percentages of effector (cluster 1, C1), exhausted (cluster 4, C4), and memory (cluster 6, C6) CD8+tumor-infiltrating lymphocytes (TIL) were significantly higher in IL-33-expressed tumors (see FIG. 1D). In addition, the exhausted cluster showed the highest expression of Ifng among all CD8 subpopulations. Flow cytometry was performed to confirm if the protein level of IFN-γ increased in IL-33 protein treated or IL-33 expressing tumor. It was found that both IL-33 protein treatment and over-expressed IL-33 in tumor cells significantly increased the percentage of IFN-γ⁺ CD8⁺ T cells in the tumor microenvironment (see FIGS. 8C and 8D).

Analysis of TCR sequences at the single-cell level led to the examination of clonal expansion in CD8⁺ TIL subsets. Notably, CD8⁺ TIL subsets from IL-33-expressing tumors showed much greater clonal expansion than those from the control tumors, except for the native cluster which consisted of only single TCR clones (see FIG. 1F). Further, the average clonal size and the total number of CD8 clones were studied. The average clonal size in all clusters was higher in the IL-33-expressing tumors compared to B16 tumors (see FIG. 1G). In addition, the total clone number was also greater in CD8⁺ TIL from B16-IL33 tumors than B16 tumors, demonstrating CD8⁺ T cells in IL-33-expressed tumors had much larger clonal diversity (see FIG. 1E). Single-cell level TCR analysis was used to track the differentiation trajectory of T cells with different antigen specificities. One clone followed the effector→exhausted→proliferating path (see FIG. 1H) and another clone traversed the effector→cytotoxic track. IL-33 led to clonal expansion and T cell functional diversification. Further, integrated analysis of single-cell-level TCRs and transcriptomes were leveraged to systematically characterize the functional diversification of clonally expanded T cells. In the control B16 tumors, only the exhausted cluster expanded, with about 20% of the clonally expanded T cells present in other clusters, mostly in the proliferating cluster (see FIGS. 1A and 1B). CD8⁺ T cells in all other clusters had little clonal expansion and functional divergence. In contrast, all B16-IL33 tumor derived cell clusters expanded, excluding the naïve cluster population, with more than 25% of clonally expanded CD8+ T cells present in multiple clusters (see FIGS. 1I, 9E, and 9F). Overall, these findings provide extensive functional diversification of CD8⁺ TIL and show that IL-33 drove robust clonal expansion.

### IL1RL1+ Treg Cells were Increased in the IL-33-Expressing Tumors

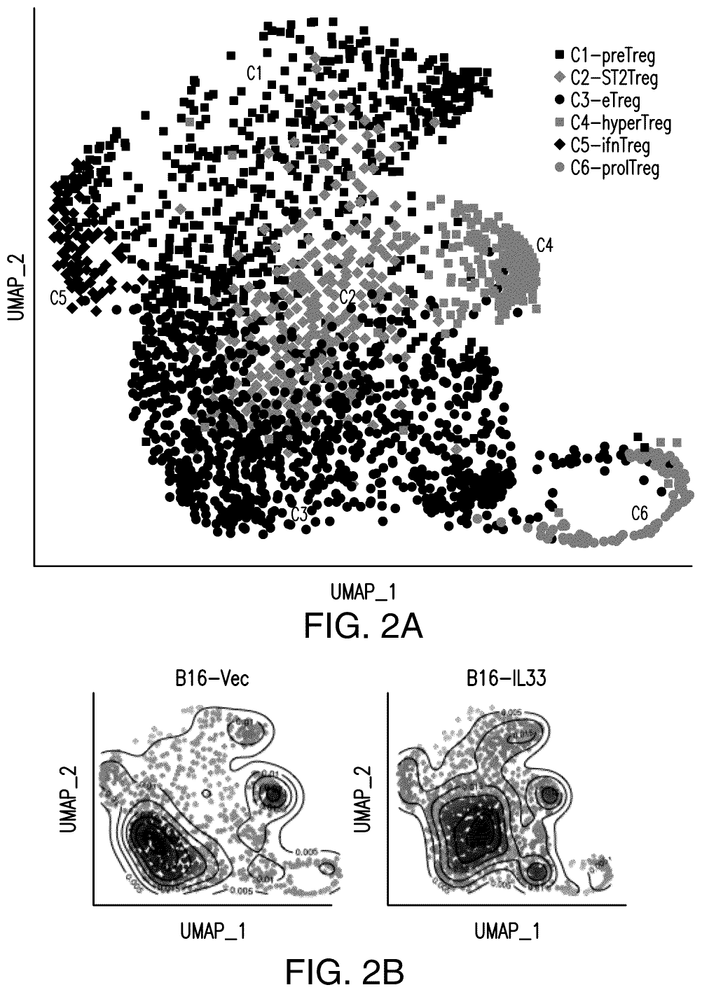

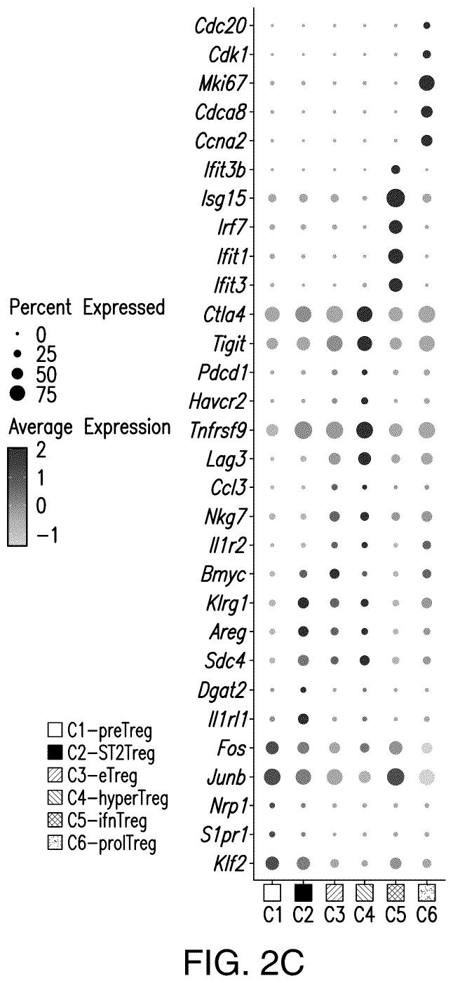

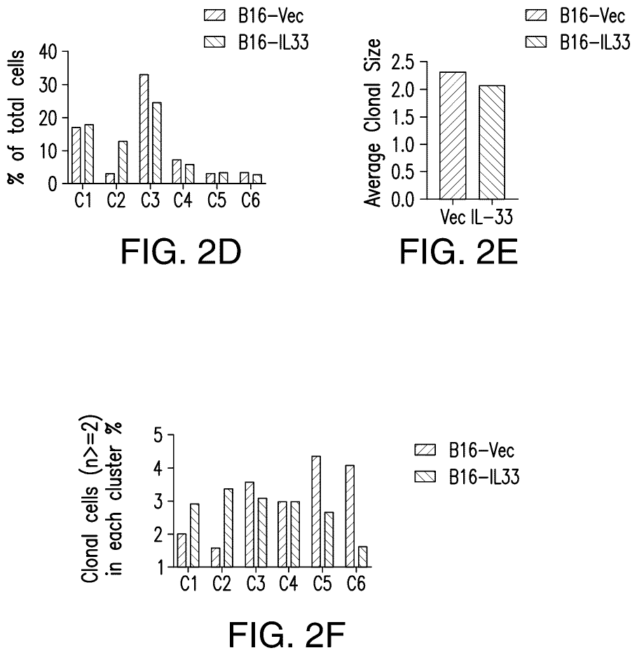

The present example shows that IL1RL1+ Treg cells were increased in the IL-33-expressing tumors. Analysis of Treg cell scRNAseq data revealed 6 clusters, namely pre-effector Treg (preTreg, cluster 1), IL1RL1+ Treg (ST2Treg, cluster 2), effector Treg (eTreg, cluster 3), hyper effector Treg (hyperTreg, cluster 4), interferon-signature Treg (ifnTreg, cluster 5), and proliferating Treg (prolTreg, cluster 6) (see FIGS. 2A-2C). The preTregs expressed the highest levels of early activation genes such as Fos, Jun, and Klf2. The eTreg and hyperTreg clusters expressed immune checkpoint molecules such as Pdcd1, Haver2, Tigit, Lag3, and Ctla4, as well as the costimulatory molecule Tnfrsf9. The ifnTregs and prolTregs expressed the highest levels of interferon-induced genes and cell proliferation-related genes respectively (see FIG. 2C). IL1RL1+ Tregs expressed a unique set of genes such as IL1rl1, Areg, Klrg1, and Sdc4 (see FIG. 2C). Notably, the fraction of IL1RL1+ Tregs was increased in the B16-IL33 tumor compared to those from the control B16 tumor (see FIGS. 2B and 2D). In contrast, the fraction of eTregs was decreased in B16-IL33 tumors (see FIG. 2D). The analysis of TCRs showed that the average clonal expansion in Treg cells was smaller than CD8+ T cells and comparable between B16 and B16-IL33 tumors (see FIG. 2E). IL-33 increased the percentage of expanded cells in the preTreg and ST2Treg. In contrast, the percentage of expanded Tregs was larger in eTreg, ifnTreg, and prolTreg clusters in B16 tumors (see FIGS. 2E). In line with the scRNA-seq data described in Example 1, IL1RL1+ Treg cells were significantly increased after treatment of IL-33 in the mouse colon cancer model MC38 (see FIGS. 10A and 10B). Overall, the present example shows that IL-33 drastically increased the proportion and clonal expansion of IL1RL1+ Treg in the TME.

### Deletion of Il1rl1 in Tregs Enhanced Anti-Tumor Immunity and Altered TME

The present example shows that specific deletion of Il1rl1 in Tregs enhanced anti-tumor immunity and altered TME. The present example investigated how IL1RL1 signaling in Treg cells regulated IL-33-stimulated antitumor immune responses. To this aim, a Foxp3^{cre}Il1rl1^{flox/flox} mice model was generated (see FIG. 3A). Foxp3^{cre}Il1rl1^{flox/flox} mice showed normal T cell development. B16-IL-33 tumor growth was initially comparable between control Foxp3^{cre}and Foxp3^{cre}Il1rl1^{flox/flox} mice. However, after around 8 days post-inoculation, the tumor progressed much faster in control Foxp3^{cre} mice than in Foxp3^{cre}Il1rl1^{flox/flox} mice (see FIG. 3B). Moreover, about 67% (6 out of 9) of the Foxp3^{cre}Il1rl1^{flox/flox} mice completely rejected the tumor starting on D12 post-inoculation (see FIGS. 3B, 12B, and 12C). In addition, the overall survival was much greater in Foxp3^{cre}Il1rl1^{flox/flox} mice (see FIG. 3C).

In addition to the B16-IL33 model, a mouse colon cancer MC38 model was used to determine the effects of IL-33 protein or ICB anti-PD-1 antibody treatment (see FIGS. 14A-14C). The MC38 tumors grew similar in control Foxp3ere mice and Foxp3^{cre}Il1rl1^{flox/flox} mice. However, upon IL-33 treatment or anti-PD-1 antibody, the Foxp3^{cre}Il1rl1^{flox/flox} mice showed a significant delay in tumor growth (see FIGS. 12D-12F). The present example shows IL1RL1 signaling in Treg cells enhanced their immune regulatory function in vivo (see FIGS. 12A-12F).

To gain mechanistic insights into the immunity, multicolor flow cytometry was performed on tumors isolated around day 8 when the tumor sizes were similar (see FIG. 11). Differences in percentages of CD45+ immune cells were identified. It was found that Treg ST2 deficiency regulated Treg cells numbers in the tumor. The number and the percentage of Treg cells were dramatically reduced in Foxp3^{cre}Il1rl1^{flox/flox} mice (see FIG. 3D). In addition, TCF-1 expression was significantly higher in Foxp3^{cre}Il1rl1^{flox/flox} Tregs (see FIG. 3E), having a more naïve-like Treg phenotype. Inhibitory receptors such as PD-1 and Tim-3 are known to be expressed on effector Treg cells, which have a more suppressive function. In line with the naïve like phenotype, the fractions of PD-1⁺, Tim-3⁺, and PD-1⁺Tim-3⁺ Treg cells were significantly decreased in the Foxp3^{cre}Il1rl1^{flox/flox} tumors (see FIG. 3F). Taken together, these results show that IL1RL1 signaling in Treg cells is important for tumor accumulation and the development of the effector Treg phenotype.

T cells in the TME were examined using flow cytometry. A significant increase in the percentage and number of CD8⁺ T cells in tumors isolated from Foxp3^{cre}Il1rl1^{flox/flox} mice was observed (see FIG. 3G). In addition, TCF-1 expression was decreased in CD8⁺ TILs from Foxp3cTeIlIrl/flox/flox mice (see FIG. 3H). Moreover, the percentage of Ki-67⁺ and GzmB⁺ cells were significantly increased in CD8⁺ TILs from Foxp3^{cre}Il1rl1^{flox/flox} mice (see FIGS. 3I and 3J). The percentage of IFN-γ⁺ CD8⁺ TILs also increased in Foxp3^{cre}Il1rl1^{flox/flox} mice. Furthermore, the co-inhibitory molecules PD-1, Tim-3, and CD39 were all higher in the CKO CD8⁺ TIL. It has been reported that PD-1⁺ Tim-3⁺ CD8⁺ TILs are hyper-activated effector T cells. These findings are in line with a more functional and effective CD8⁺ T cell-dominated TME in Foxp3^{cre}Il1rl1^{flox/flox} mice (see FIGS. 11G and 11H). Next, the number of infiltrating ILC2 cells in the B16-IL33 tumors was determined. The percentage of infiltrating ILC2 cells increased in the Foxp3^{cre}Il1rl1^{flox/flox} mice compared to control mice (see FIG. 3K). Thus, genetically ablation of Il1rl1 in Treg cells inhibited its suppressive function and improved CD8⁺ T cell-mediated type 1 anti-tumor immunity.

Next, tumor-associated myeloid populations were investigated and it was found that the fraction of CD11b⁺ myeloid cells was significantly decreased in tumors isolated from Foxp3^{cre}Il1rl1^{flox/flox} mice (see FIG. 4A). A more granular look at the myeloid-derived suppressor cell (MDSC) populations showed that the fraction of granulocytic MDSC (gMDSC) was comparable between control and Foxp3^{cre}Il1rl1^{flox/flox} mice, whereas the frequency of monocytic MDSC (mMDSC) was significantly reduced in tumors from Foxp3^{cre}Il1rl1^{flox/flox} mice (see FIG. 4B). A less immunosuppressive TME in Foxp3^{cre}Il1rl1^{flox/flox} mice was also observed. Further, MHC-II expression was significantly elevated in mMDSCs from Foxp3^{cre}Il1rl1^{flox/flox} mice (see FIG. 4C). Within the dendritic cell population (DC), the percentage of CD11b+CD11c+DCs were significantly increased in the TME of Foxp3^{cre}Il1rl1^{flox/flox} mice (see FIG. 4D). In addition, among the DCs, the fraction of CD103⁺ DCIs was around two-fold higher in tumors from Foxp3^{cre}Il1rl1^{flox/flox} mice than control mice (see FIG. 4E). Flow cytometric analysis also revealed that the fraction and number of tumor associate macrophages (TAM) decreased in Foxp3^{cre}Il1rl1^{flox/flox} mice (see FIG. 4F). Further, the fraction of type 2 tumor-associated macrophages (M2) decreased and the number of M2 was significantly lower in the TME of Foxp3^{cre}Il1rl1^{flox/flox} mice (see FIG. 4G). Collectively, these data show a more immunogenic TME in the Foxp3eTell1rl/flox/flox mice.

### IL1RL1 signaling is required for IL-33-driven accumulation of IL1RL1+ Tregs in the tumor

The present example shows that IL1RL1 signaling is required for IL-33-driven accumulation of IL1RL1+ Tregs in the tumor. The mechanisms underlying IL-33-driven Treg-mediated immune suppression were investigated by scRNA-seq analysis of tumor-associated Treg cells from both control and Foxp3^{cre}Il1rl1^{flox/flox} (see FIGS. 5A and 5B). The analysis revealed that the frequency of IL1RL1+ Treg cells was diminished but the frequencies of preTreg and eTreg were increased in the tumor from Foxp3^{cre}Il1rl1^{flox/flox} compared to control Foxp3^{cre} mice (see FIG. 5C). The clonal expansion was reduced in IL1RL1+ Tregs but increased in preTreg, eTreg, and ifn Treg cells (see FIG. 5D). These data suggest that IL1RL1 signaling in Treg cells is crucial for the accumulation and clonal expansion of IL1RL1+ Treg cells in tumors expressing secreted IL-33.

An in-depth scRNA-seq analysis was performed to gain insight into the phenotype and role of the infiltrating Treg cells, and how they contribute to the IL-33 response. Key driver transcription factors that can regulate IL1RL1+ Treg differentiation were identified. Single-cell regulatory network inference and clustering (SCENIC) analysis revealed several essential transcription factor regulons differentially expressed in IL1RL1+ Treg cells between control mice and Foxp3^{cre}Il1rl1^{flox/flox} mice. Bcl3 was identified as a molecule of interest. Bc13 is an atypical member of the NF-κB family that acts in the nucleus to regulate the transcription of many NF-κB target genes. Further, several NF-κB binding sites are also enriched in the Bcl3 target genes in IL1RL1+ Treg cells (see FIG. 5E). Bc13 regulon activity was restricted to the IL1RL1+ Treg cluster and much higher in Treg cells from a control than those from the Foxp3^{cre}Il1rl1^{flox/flox} mice (see FIG. 5F and 5G). Interestingly, NF-κB binding sites are also enriched in the Nfkb2 target genes (see FIG. 5H). Interestingly, Nfkb2 showed exclusive activities in the IL1RL1+ Treg cluster (see FIG. 5I). In addition, Nfkb2 regulon activity was significantly higher in control Treg cells (see FIG. 5J). In addition to NF-κB associated transcription factors, it was found that the Stat 1 regulon was also specifically upregulated in IL1RL1+ Treg cells and its regulon activity was dependent on IL1RL1 signaling Maf (see FIGS. 13A-13C).

### AREG/EGFR Crosstalk Between IL1RL1+ Tregs and CAFs Drives Tumor Immune Suppression

The present example shows AREG/EGFR crosstalk between IL1RL1+ Tregs and CAFs drives tumor immune suppression.

Differential expressed genes were examined between CON(Foxp3crc) and CKO (Foxp3^{cre}Il1rl1^{flox/flox}) mice, and in response to IL-33 treatment (see FIGS. 6E and 6F). Amphiregulin (Areg) is uniquely expressed in IL1RL1+ Treg cells and is an important effector molecule for tissue repair. The present example found that Areg was predominantly expressed in IL1RL1+ Treg cells in B16-IL33 tumors (see FIG. 2C). Areg expression was compared in Treg cells among tumor-bearing control mice and Foxp3^{cre}Il1rl1^{flox/flox} mice and it was found that Areg was most predominantly expressed in IL1RL1+ Treg cells (see FIG. 6A-6C). In addition, the AREG gene expression was greatly reduced in Treg cells in the Foxp3^{cre}Il1rl1^{flox/flox} mice (see FIG. 6A). Analysis of ATAC-seq results also revealed that the Areg locus was more accessible in IL1RL1+ Treg cells compared with other Treg cells (see FIG. 6B). Thus, Treg-expressed Areg plays a role in IL-33-driven antitumor immune responses.

Areg protein is a member of the epidermal growth factor (EGF) family and interacts with the Epidermal growth factor receptor (EGFR) to trigger its downstream cascades. After analyzing whole tumor scRNA-seq data, it was found that EGFR was predominantly expressed in cancer-associated fibroblasts (CAF) (see FIG. 6D). EGFR was expressed by tumor cells as well, although at a lower level than CAF. In addition, there was no evidence of EGFR expression in T cells, NK cells, myeloid cells, or other immune cell types in the TME. This pattern of EGFR expression was also confirmed using scRNA-seq datasets from mouse melanoma tumors and patients with pancreatic ductal adenocarcinoma (PDAC) (see FIG. 15A-15D). These data indicated that CAFs were the major source of EGFR in the TME and that the Areg/EGFR axis coupled IL1RL1+ Treg cells with CAFs in the TME.

The present example also shows that Areg is involved in regulating IL-33-driven antitumor immunity. B16-IL33 tumor-bearing control mice and Foxp3^{cre}Il1rl1^{flox/flox} mice were treated with an anti-AREG antibody. Anti-AREG antibody treatment significantly inhibited tumor growth in control mice but did not further inhibit tumor growth in Foxp3^{cre}Il1rl1^{flox/flox} mice (see FIG. 6G). Areg suppresses the antitumor efficacy of IL-33 and activity is dependent on IL33 signaling on Treg cells. Indeed, Areg produced by cells other than IL1RL1+ Tregs was not required for the immune inhibitory function. Therefore, these data demonstrate a regulatory role of Areg expressed by IL1RL1+ Treg in IL-33-driven antitumor immune responses.

Clinical implications of activated tumor Tregs induced by IL-33 and differentially genes expressed in ST2Treg cells.

The present example shows the clinical implication of activated tumor Tregs induced by IL-33 and differentially genes expressed in ST2Treg cells between control mice and Foxp3^{cre}Il1rl1^{flox/flox} mice. In the present example, nHDP modeling was applied to scRNA analysis to identify gene expression modules (GEMs) that were regulated by ST2 signaling. When applied to scRNA data, nHDP searches for modules of genes that exhibited coordinated expression in single cells, such that genes in a GEM were likely regulated by a common regulatory pathway, and that the presence and absence in a single cell reflect the state of the pathway. The nHDP model was applied to the mouse scRNA-seq data including a mix of Treg cells from control mice and Foxp3^{cre}Il1rl1^{flox/flox} mice. Notably, the analysis discovered 85 GEMs among the cells. Next, it was examined whether any GEM (as a collection rather than individual genes) was differentially expressed between control and ST2 deficient cells. It was found that GEM16 was significantly depressed in the ST2KO cell population (p-value=2.2E-16). The top rank genes of this GEM included markers of Treg cells and some hallmarks of a certain process of Treg cells. Among the top 50 genes of this GEM, 15 (see FIG. 7A) were also deemed as DEGs from supervised statistical analysis. Interestingly, the gene IL1RL1 (encoding ST2) was among them, indicating that nHDP had correctly captured their coordinated expression status. GEM16 represents a signature of a subpopulation of Tregs, whose expression was regulated by IL33/ST2 signaling.

### IL33 Plays a Role in Regulating the Function of a Subpopulation of Tregs Through the ST2 Pathway

The present example shows that IL33/ST2-mediated signaling in tumors by investigating the role the IL33/ST2 signaling axis plays in different human cancers. Specifically, the following causal chain was investigated:
expression of IL33→formation of IL33-ST2 signal→expression of GEM16 functions in different cancers.

From the viewpoint of statistical causal inference, the present example assessed the above causal relationship through testing whether the expression of GEM16 and IL33 were statistically independent conditioning on a variable that represented the ligand-receptor interaction.

From the TCGA database, bulk RNA expression data were collected for 6 cancer types of interest (see FIG. 7A). For each tumor, the expression values of IL33 and ST2 were extracted, and the overall expression of GEM16 was estimated in tumors using the deconvolution methods referred to as GSVA analysis. FIG. 7A shows that the expression of IL33 and GEM16 were significantly correlated in multiple cancer types except in COAD and GBM. FIGS. 7A and 7B also show that the expression of ST2 was more strongly correlated with expression values of

GEM16. The results of marginal correlations showed that expression of IL33 causally influenced the expression of GEM16.

Conditional independence testing was used to further investigate the causal relation stated above. The product of expression values of IL33 and IL1RL1 was used to approximate the concentrate of the IL33 and ST2 proteins. Mimicking the Hill-Langmuir equation, the concentration of ligand-receptor protein complex was estimated as follows:

[IS]∝[IL33]*[ST2],

where [IS] is the approximated concentration of ligand-receptor protein complex, and

[IL33] and [ST2] are mRNA expression values of IL33 and ILIRLI, which served as surrogates for the protein concentrations of IL33 and ST2, respectively.

Next, it was assessed whether the expression of IL33 causally influences the expression of GEM16 in tumors through a conditional independence test. FIG. 7A shows that conditioning on [IS], expression values of IL33 and GEM16 were independent in several cancer types. The present example shows that IL33 plays a role in regulating the function of a subpopulation of Tregs through the ST2 pathway and that the members of the ST2-regulated genes can play downstream signaling roles in modulating the tumor environment.

Collectively, these findings provided fundamental insight into the complex interplay among cells within the TME and highlight the immunosuppressive role CAFs and their interaction with Treg cells and infiltrating myeloid cells.

### Discussion

The present disclosure demonstrated that IL-33 expression in tumor cells resulted in robust CD8+ T cell-mediated antitumor immune responses characterized by massive CD8+ T cell clonal expansion and functional diversification. However, IL-33 also promoted Treg cell accumulation in the TME particularly the IL1RL1+ subset. The present disclosure further demonstrated that IL1RL1 signaling was required for the accumulation of IL1RL1+ Treg in the TME and the immune suppressive function of IL1RL1+ Tregs during IL-33-driven antitumor immunity. At the mechanistic level, the Areg/EGFR axis mediated crosstalk between Treg cells and CAFs in the TME. Further, the present disclosure found that Areg mediated the immune regulatory function and impeded the antitumor efficacy of IL-33.

The present disclosure demonstrated that Areg mediated immune suppression by IL1RL1+ Treg cells. However, the reported role of IL1RL1+ Treg cells is complex. Evidence showed that the IL1RL1+ Treg subtype exerted strong immune suppressive and anti-inflammatory functions in would repair, tissue homeostasis, autoimmunity, and cancer. In addition, IL1RL1+ Treg cells were also involved in muscle and lung tissue repair after injury. Areg has been assigned different roles depending on cellular origin. For example, Areg produced by mast cells and basophil cells have been found to regulate immune responses by promoting EGFR signaling in Treg cells. The present disclosure found that Treg cells did not express EGFR. Areg produced by IL1RL1+ Treg cells is thought to be an important effector molecule for tissue repair. The present disclosure discovered a new mechanism by which Areg regulates antitumor immune response. The present disclosure also shows that Areg inhibited the antitumor efficacy of IL33 treatment and that EGFR was specifically expressed in CAFs but not Treg cells in the TME. In addition, both IL33 expression and Treg-specific Il1rl1 deletion affected the MHC-II and chemokine expression in CAFs. Thus, Areg/EGFR axis coupled IL1RL1+ Treg and CAFs together, thereby promoting immune tolerance in the TME.

To date, there are at least three major subtypes of CAFs cells, namely myofibroblast (myCAF), inflammatory CAF (iCAF), and antigen presentation CAF (apCAF). TGFβ1 is known to drive myCAF differentiation while ILI is known to drive iCAF differentiation. The cytokines that drive the apCAF subset are less clear. It is possible that MHC-II, like PD-L1, is up-regulated by IFN-γ produced by Type 1 lymphocytes. It has been proposed that MHC-II is involved in promoting the dysfunction of conventional CD4+ T cells in the TME. It is also likely that the CAF-expressed self-antigen peptide-MHC-II complexes engage and stimulate Treg cells in the TME. Other than MHC-II, immune signals such as B7-H3 and PD-L2 have been shown to allow CAFs to control Treg cells. In addition, IL33 is produced by fibroblasts in many pathological settings such as colitis, and adipose tissue inflammation, and inhibited over-zealous inflammation as well as promote tissue repair. CAFs can also produce IL33, which promotes tumor metastasis and immune suppression through Treg and myeloid cells.

The present disclosure determined that Treg can also influence the fate of CAFs cells through AREG/EGFR interaction. This interaction resulted in more robust proliferation, survival, and even development of immunosuppressive characteristics. More importantly, AREG/EGFR enabled the coupling of Treg and CAF and allowed greater interdependence of this cellular module.

Such coupling played an important role in shaping immune suppression in the TME. Notably, decoupling with Areg-antibody improved the antitumor efficacy of IL33. Overall, the present example shows that Treg was a key player in driving the development of an immune suppressive phenotype in CAFs.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A pharmaceutical composition comprising an IL33 polypeptide and an anti-AREG antibody or a fragment thereof for use in treating and/or preventing a cancer in a subject in need thereof.

2. The pharmaceutical composition of claim 1, wherein the IL33 polypeptide comprises an amino acid sequence at least about 80% identical to the amino acid sequence set forth in SEQ ID NO: 1.

3. The pharmaceutical composition of claim 2, wherein the IL33 polypeptide comprises the amino acid sequence set forth in SEQ ID NO: 1.

4. The pharmaceutical composition of claim 1, wherein the IL33 polypeptide comprises an amino acid sequence comprising from amino acid 95 to amino acid 270 of SEQ ID NO: 1.

5. The pharmaceutical composition of claim 1, wherein the IL33 polypeptide comprises an amino acid sequence comprising from amino acid 99 to amino acid 270 of SEQ ID NO: 1.

6. The pharmaceutical composition of claim 1, wherein the IL33 polypeptide comprises an amino acid sequence comprising from amino acid 109 to amino acid 270 of SEQ ID NO: 1.

7. The pharmaceutical composition of claim 1, wherein the IL-33 polypeptide is encoded by a vector.

8. The pharmaceutical composition of any one of claim 1, wherein the subject has received or is receiving an immunomodulatory agent.

9. A method for treating and/or preventing a cancer or a tumor in a subject in need thereof, comprising administering a therapeutically effective amount of an IL33 polypeptide and an anti-AREG antibody or a fragment thereof to the subject.

10. The method of claim 9, wherein the IL33 polypeptide comprises an amino acid sequence at least about 80% identical to the amino acid sequence set forth in SEQ ID NO: 1.

11. The method of claim 9, wherein the IL33 polypeptide comprises the amino acid sequence set forth in SEQ ID NO: 1.

12. The method of claim 9, wherein the IL33 polypeptide comprises an amino acid sequence comprising from amino acid 95 to amino acid 270 of SEQ ID NO: 1.

13. The method of claim 9, wherein the IL33 polypeptide comprises an amino acid sequence comprising from amino acid 99 to amino acid 270 of SEQ ID NO: 1.

14. The method of claim 9, wherein the IL33 polypeptide comprises an amino acid sequence comprising from amino acid 109 to amino acid 270 of SEQ ID NO: 1.

15. The method of claim 9, wherein the IL-33 polypeptide is encoded by a vector.

16. The method of claim 9, wherein the cancer is selected from the group consisting of adenocarcinomas, osteosarcomas, cervical carcinomas, melanomas, hepatocellular carcinomas, breast cancers, lung cancers, prostate cancers, ovarian cancers, leukemia, lymphomas, renal carcinomas, pancreatic cancers, gastric cancers, colon cancers, duodenal cancers, glioblastoma multiforme, astrocytomas, sarcomas, and combinations thereof.

17. The method of claim 9, further comprising administering an immunomodulatory agent to the subject.

18. The method of claim 9, wherein the tumor is resistant to treatment with said IL33 polypeptide or functional fragment thereof when administered as a single agent.

19. The method of claim 9, wherein the tumor is an IL-33 positive tumor.

20. A kit for use in treating and/or preventing a cancer in a subject in need thereof, wherein the kit comprises an IL33 polypeptide and an anti-AREG antibody or a fragment thereof.
